(19) United States
(12) Patent Application Publication
(10) Pub. No.: US 2013/0261178 A1
(43) Pub. Date: Oct. 3, 2013

(54) PHENOXYPROPANOL DERIVATIVES AND THEIR USE IN TREATING CARDIAC AND CARDIOVASCULAR DISEASES

(71) Applicants: Shailesh Mistry, Nottingham (GB); Etienne Daras, Nottingham (GB); Christophe Fromont, Nottingham (GB); Gopal Jadhav, Nottingham (GB); Peter Martin Fischer, Beeston (GB); Barrie Kellam, Funtham (GB); Stephen John Hill, Nottingham (GB); Jillian Glenda Baker, Nottingham (GB)

(72) Inventors: Shailesh Mistry, Nottingham (GB); Etienne Daras, Nottingham (GB); Christophe Fromont, Nottingham (GB); Gopal Jadhav, Nottingham (GB); Peter Martin Fischer, Beeston (GB); Barrie Kellam, Funtham (GB); Stephen John Hill, Nottingham (GB); Jillian Glenda Baker, Nottingham (GB)

(21) Appl. No.: 13/808,493

(22) PCT Filed: Jul. 5, 2010
(86) PCT No.: PCT/GB10/51106

§ 371 Date: Jun. 17, 2013

(51) Int. Cl. C07C 217/64; C07C 231/12; C07C 275/30; C07C 275/36; C07C 273/18; C07D 303/24; C07C 271/28; C07C 269/06; C07C 335/20; C07C 311/13; C07C 303/40; C07C 43/23; C07C 235/34; C07C 213/08

(52) U.S. Cl. CPC C07C 217/64; C07C 235/34; C07C 231/12; C07C 275/30; C07C 275/36; C07C 273/1854; C07C 213/08; C07C 271/28; C07C 269/06; C07C 335/20; C07C 311/13; C07C 303/40; C07C 43/23; C07D 303/24
USPC 514/488; 564/170; 514/622; 564/53; 514/596; 514/598; 564/52; 564/349; 514/652; 560/32; 564/27; 514/586; 564/92; 514/604; 568/644; 549/560; 568/649; 568/650

(57) ABSTRACT

A compound of formula I-0, and its pharmaceutically acceptable salt or salts and physiologically hydrolysable derivatives in free form or salt form:

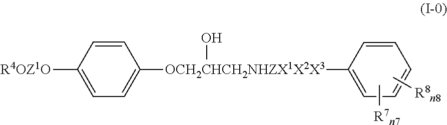

wherein
Z¹ is C₁-C₄ linear or branched alkyl or alkenyl;
R⁴ is selected from unsubstituted and substituted C₃-C₃ cycloalkyl, C₁-C₈ linear or branched alkyl, C₂₋₅ alkenyl, C₆-C₁₀ heteroaryl or aryl, or C₃-C₈ heterocyclyl which may be part unsaturated, and combinations thereof;
Z is linear C₂₋₃ alkylene;
X¹ is selected from NH and O;
X² is selected from unsaturated C and unsaturated S; and
X³ is selected from NH and CH₂; or
one of X¹ and X³ is a single bond; or
X¹ is O and X² and X³ together are a single bond; and
R⁷ is selected from oxo, F, Cl, Br, CN, NH₂, NR⁹₂, NO₂, CF₃, OR⁹, COR⁹, OCOR⁹, COOR⁹, NR⁹COR⁹, CONR⁹₂SO₂NR⁹₂, NR⁹SO₂R⁹; and
R⁸ is selected from C₁₋₅ alkyl, C₁₋₅ alkoxyl, C₂₋₅ alkenyl or alkynyl, C₆₋₁₀ aryl and C₃₋₈ cycloalkyl and combinations thereof, which may be unsubstituted or further substituted by one or more F, Cl, Br, CN, NH₂, NR³₂, NO₂, CF₃; and
R⁹ is selected from H and a group R⁸ as hereinbefore defined;
n7 and n8 and the sum thereof are independently selected from zero and the whole number integer 1 to 4;
   processes for the preparation thereof, compositions and uses.

This invention relates to novel compounds and their preparation and use in treating cardiac and cardiovascular disease.

### BACKGROUND

Beta-adrenoceptor antagonists (beta-blockers) are one of the most important therapies in the management of symptoms of, and for prolonging life in, cardiovascular disorders e.g. ischaemic heart disease and cardiac arrhythmias. They work by blocking the beta1-adrenoceptors in the heart and thus prevent the endogenous hormones adrenaline and noradrenaline from increasing heart rate and force of contraction. Beta-blockers are also widely used in the management of hypertension, and (although the mechanism of action is not yet understood) they prolong life in patients with heart failure.

However, they are contraindicated in patients with respiratory disease (especially asthma and chronic obstructive pulmonary disease, COPD) because antagonism of the beta2-adrenoceptors in the airways results in bronchoconstriction and a loss of action of the important beta2-agonist bronchodilators. Thus, currently many people (about 0.6% of the total adult population in the UK) with cardiovascular disease are unable to take beta-blockers that would prolong their life and improve their cardiovascular symptoms, because of their concomitant respiratory disease. This is because the best beta1-selective beta-antagonist currently available for clinical use binds to the human beta1-adrenoceptor with only 14 fold higher affinity than the human beta2-adrenoceptor (Baker, 2005; Br. J Pharmacol: 144, 317-22).

Accordingly there is a need for beta blockers which are selective for just heart disease, ie have a high beta₁/beta₂ selectivity. Classes of phenoxypropanolamine compounds are known which are extended beyond the amine group and are substituted in the phenol ring. One particular class of phenoxypropanolamine compounds comprises a substituted ethylene dioxy substituent para to the phenyl moiety. This class which has never entered into clinical use includes the development compound LK-204545 with an phenyl(alkylurea) substituent to the amine moiety and with 1.778-fold β₁-selectivity:

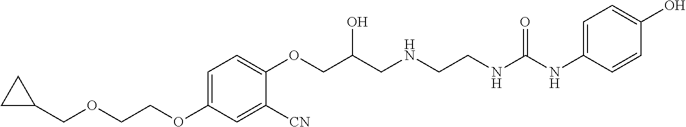 [SMILES: N#Cc1cc(OCCOCC2CC2)ccc1OCC(O)CNCCNC(=O)Nc1ccc(O)cc1]

and D-140S with a phenyl alkyl substituent to the amine moiety and with 4.400-fold β₁-selectivity:

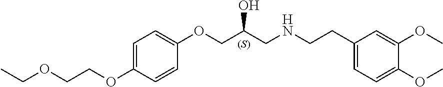

WO2008083054 discloses beta-1 adrenoreceptor selective ligands that find use as imaging agents within nuclear medicine applications. Compounds include an imaging moiety such as a radioactive moiety. The broadly disclosed class of compounds includes compounds having the core 1-phenoxy, 2-hydroxy propan-3-amine with extensive substitution of the phenoxy and amine moieties.

### BRIEF SUMMARY OF THE DISCLOSURE

We have now applied a multidisciplinary approach to beta receptor agonist and antagonist design to provide novel compounds which have significant selectivity for beta-1 adrenoceptors and which have potential for clinical use.

### BRIEF DESCRIPTION OF THE DRAWINGS

Embodiments of the invention are further described hereinafter with reference to the accompanying drawings, in which:

Schemes 1 and 2 illustrate the preparation of intermediate compounds and

Schemes 3 and 4 illustrate the preparation of compounds of formula I.

### DETAILED DESCRIPTION

In accordance with the present invention there is provided a compound of formula I, and its pharmaceutically acceptable salt or salts and physiologically hydrolysable derivatives in free form or salt form:

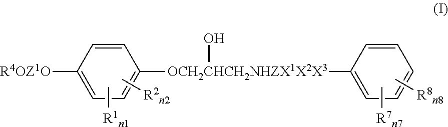

wherein
R⁴ is selected from unsubstituted and substituted C₃-C₈ cycloalkyl which may be part unsaturated, C₁-C₈ linear or branched alkyl, C₂₋₅ alkenyl, C₆-C₁₀ heteroaryl or aryl, and C₃-C₈ heterocyclyl which may be part unsaturated, and combinations thereof;
Z¹ is C₁-C₄ branched or linear alkyl or alkenyl;
n1 and n2 and the sum thereof are independently selected from zero and the whole number integer 1 to 4, and;
R¹ is selected from F, Cl, Br, CN, NH₂, NO₂, CF₃, OR³, COR³, OCOR³, COOR³, NR³COR³, CONR³₂ SO₂NR³₂, NR³SO₂R³; and
R² is selected from C₁₋₅ alkyl, C₁₋₅ alkoxyl, C₂₋₅ alkenyl or alkynyl, C₈₋₁₀ aryl and C₃₋₈ cycloalkyl and combinations thereof any of which may comprise one or more carbonyl units or heteroatoms selected from O, S and N, and which may be part unsaturated, and which may be unsubstituted or further substituted by any of the foregoing substituents R¹ as hereinbefore defined; and
R³ is selected from H and a group R² as hereinbefore defined;
Z is linear C₂₋₃ alkylene, optionally substituted by OH;
X¹ is selected from NH and O;
X² is selected from unsaturated C and unsaturated S; and
X³ is selected from NH and CH₂; or
one of X¹, X² and X³ is a single bond; or
X² and X³ are each a single bond; and
R⁷ is selected from F, Cl, Br, CN, NH₂, NR⁹₂, NO₂, CF₃, OR⁹, COR³, OCOR⁹, COOR⁹, NR⁹COR⁹, CONR⁹₂ SO₂NR⁹₂, NR⁹SO₂R⁹; and
R⁸ is selected from C₁₋₅ alkyl, C₁₋₅ alkoxyl, C₂₋₅ alkenyl or alkynyl, C₆₋₁₀ aryl and O₃₋₈ cycloalkyl and combinations thereof, which may be unsubstituted or further substituted by one or more F, Cl, Br, CN, NH₂, NR⁹₂, NO₂, CF₃; and
R⁹ is selected from H and a group R⁸ as hereinbefore defined;
n7 and n8 and the sum thereof are independently selected from zero and the whole number integer 1 to 4;
   with the proviso that it is not a compound as listed in the following Tables:

TABLE A1

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| CH₃ | CH₂CH₂ | — | CH₂CH₂ | NH | CO | NH | — |
| | | o-CN | | | | — | |
| | | o-Br | | | | | |
| | | o-Cl | | | | | |
| | | o-CH₂CH═CH₂ | | | | | |
| | | (allyl) | | | | | |
| | | o-NHCOCH₃ | | | | | |
| | | o-NHCOOCH₃ | | | | | |
| | | o-COCH₃ | | | | | |
| | | o-CONH₂ | | | | | |
| | | o-pyrrole | | | | | |
| | | m-CN | | | | | |
| | | m-Br | | | | | |
| | | m-NHCOCH₃ | | | | | |
| CH₃ | CH₂CH₂ | o-CN | CH₂CH₂ | NH | CO | CH₂ | — |
| | | o-Br | | | | | p-OH |
| CH₃ | CH₂CH₂ | o-CN | CH₂CH₂ | NH | CO | NH | p-CH₂CH₂OCH₃ |
| | | o-Br | | | | | p-CN |
| | | | | | | | p-OH |
| | | | | | | | m-CF₃ |
| | | | | | | | m-OCH₃ |
| | | | | | | | o,o-(CH₃)₂ |
| CH₃ | CH₂CH₂ | o-CN | CH₂CH₂ | NH | CO | NH | p-OCH₃ |
| | | o-Br | | | | — | |
| CH₃ | CH₂CH₂ | o-CN | CH₂CH₂ | NH | CO | NH | p-CH₃ |
| | | | | | | | p-CONH₂ |
| | | | | | | | o-OCH₃ |
| CH₃ | CH₂CH₂ | o-CN | CH₂CH₂ | NH | CO | — | p-NH₂ |
| CH₃ | CH₂CH₂ | o-Br | CH₂CH₂ | NH | CO | NH | o-CF₃ |
| CH₃ | CH₂CH₂ | o-CN | (CH₂)₃ | NH | CO | NH | — |
| c.prCH₂ | CH₂CH₂ | — | CH₂CH₂ | NH | CO | NH | — |
| | | o-CN | | | | | |
| | | o-Br | | | | | |
| c.prCH₂ | CH₂CH₂ | o-CN | CH₂CH₂ | NH | CO | NH | p-OH |
| | | (LK204545) | | | | — | |
| | | o-Br | | | | | |
| c.prCH₂ | CH₂CH₂ | o-CN | CH₂CH₂ | NH | CO | NH | m-OH |
| | | o-Br | | | | | o-OH |
| | | | | | | | m,p-(OH)₂ |
| c.prCH₂ | CH₂CH₂ | o-CN | CH₂CH₂ | NH | CO | NH | p-CONH₂ |
| | | | | | | | p-CH₂OH |
| | | | | | | | p-CH₃ |
| | | | | | | | p-Cl |
| | | | | | | | m-CN,p-OH |
| | | | | | | | m-OH,p-CN |
| | | | | | | | m-CONH₂ |
| | | | | | | | m-carbamoyl,p-OH |
| | | | | | | | m-OH,p-carbamoyl |
| p- | CH₂CH₂ | — | CH₂CH₂ | NH | CO | NH | — |
| OCH₃PhCH₂ | | | | | | | |
| i.pr | CH₂CH₂ | o-CN | CH₂CH₂ | NH | CO | NH | — |
| | | | | | | | p-OH |
| Ph | CH₂CH₂ | o-CN | CH₂CH₂ | NH | CO | NH | p-OH |
| i.bu | | | | | | | |

TABLE A2

| | | R¹ₙ₁, | | | | | |
|---|---|---|---|---|---|---|---|
| R⁴ | Z¹ | R²ₙ₂ | Z | X¹ | X² | X³ | R⁷ₙ₇, R⁸ₙ₈ |
| c.prCH₂ | CH₂CH₂ | m-Br | CH₂CH₂ | NH | CO | NH | p-OH |

TABLE A3

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| C₁₋₄ alkyl, | CH₂CH₂ | — | C₂₋₅alkylene | O | — | — | o-OH, m-CONR₂ |
| C₁₋₇ alkyl, | | Subst. | (eg CH₂CH₂) | | | | o-CONR₂, m-OH |
| C₁₋₄ | | | | | | | m-OH, p-CONR₂ |
| c.alkyl, C₁₋₇ | | | | | | | m-CONR₂, p-OH |
| c.alkyl, | | | | | | | where R is H or any |
| (e.g. CH₃, | | | | | | | hydrocarbyl |
| c.prCH₂) | | | | | | | eg CONH₂CONHiBu or NR₂ |
| | | | | | | | may form a cyclic moiety. |

TABLE A4

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| c.prCH₂ | CH₂CH₂ | — | CH₂CH₂ | O | — | — | p-OCH₃ |
| | | o-CN | | | | | |
| p-CH₃OBz | CH₂CH₂ | — | CH₂CH₂ | O | — | — | p-OCH₃ |
| | | o-Br | | | | | |
| CH₃ | CH₂CH₂ | — | CH₂CH₂ | O | — | — | p-OCH₃ |
| CH₃ | CH₂CH₂ | — | CH₂CH₂ | O | — | — | m,p-(OCH₃)₂ |
| | | o-Br | | | | | |
| CH₃ | CH₂CH₂ | o-Br | CH₂CH₂ | O | — | — | p-OCH₃ |
| CH₃ | CH₂CH₂ | — | CH₂CH₂ | O | — | — | — |
| CH₃ | CH₂CH₂ | o-CONH₂ | CH₂CH₂ | O | — | — | p-OCH₃ |
| CH₃ | CH₂CH₂ | o-Br | CH₂CH₂ | O | — | — | — |
| CH₃ | CH₂CH₂ | o-CN | CH₂CH₂ | O | — | — | — |
| c.prCH₂ | CH₂CH₂ | o-Br | CH₂CH₂ | O | — | — | — |
| | | | | | | | p-OCH₃ |
| CH₃ | CH₂CH₂ | — | CH₂CH₂ | O | — | — | m-CN, p-OH |
| CH₃c.pr | CH₂CH₂ | o-CN | CH₂CH₂ | O | — | — | p-OCH₃ |
| C₁₋₅alkyl | CH₂CH₂ | o-CN | CH₂CH₂ | O | — | — | p-OCH₃ |
| (eg CH₃ | | o-halo | | | | | |
| i.bu | | | | | | | |
| c.prCH₂) | | | | | | | |

TABLE A5

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| c.prCH₂ | CH₂CH₂ | o-CN | CH₂CH₂ | NH | CO | CH₂ | p-OH |
| Ph | CH₂CH₂ | o-CN | CH₂CH₂ | NH | CO | NH | — |
| CH₃ | CH₂CH₂ | o-CN | CH₂CH₂ | NH | CO | NH | m-OH |
| c.prCH₂ | CH₂CH₂ | o-Br | CH₂CH₂ | NH | CO | CH₂ | p-OH |
| CH₃ | CH₂CH₂ | m-NHCOCH₃ | CH₂CH₂ | NH | CO | NH | — |

TABLE A6

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| CH₃ | CH₂CH₂ | — | CH₂CH₂ | O | — | — | m-CONH₂, p-OH |
| CH₃ | CH₂CH₂ | o-allyl | CH₂CH₂ | O | — | — | p-OCH₃ |
| p-CH₃OPhCH₂ | CH₂CH₂ | o-CN | CH₂CH₂ | O | — | — | p-OCH₃ |
| c.prCH₂ | CH₂CH₂ | — | CH₂CH₂ | O | — | — | m,p-(OCH₃)₂ |
| c.prCH₂ | CH₂CH₂ | o-allyl | CH₂CH₂ | O | — | — | p-OCH₃ |
| i.pr | CH₂CH₂ | o-CN | CH₂CH₂ | O | — | — | p-OCH₃ |

In the above Tables:

Table A1 refers to EP52072 & CAPLUS Registry compounds
Table A2 refers to J Med Chem 2006 49(12)3467-3477(CGP20712A)
Table A3 refers to U.S. Pat. No. 4,363,6511, (=U.S. Pat. No. 4,497,813) & U.S. Pat. No. 4,363,6511 & a CAPLUS Registry compound
Table A4 refers to GB2132611, WO97/13744 and CAPLUS Registry Compounds
Table A5 refers to CAPLUS Registry compounds
Table A6 refers to B J Clin Pharm (1989) 27(5), 553-561 (trigevolol) and CH664559 and CAPLUS Registry compounds

In the tables and hereinbelow alternative substituents are indicated on sequential lines without intervening punctuation and combinations of substituents are indicated on a single line separated by a comma; where more than one integer are shown as having alternative substituents, as in Table A1 first entry, then all combinations are included, eg R¹ₙ₁, R²ₙ₂ is absent and X³ is NH, and R¹ₙ₁, R²ₙ₂ is absent and X³ is a single bond; and abbreviations have the following meanings c. is cyclo; i. is iso; pr. is propyl; bu is butyl; pent is pentyl; halo is F, Cl, Br or I; Ph is phenyl and Bz is benzyl; o, m and p are ortho, meta and para.; subst. is substituted; o.s. is optionally substituted; - is unsubstituted.

Preferably

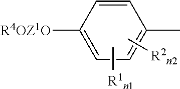

is selected from p-cyclopentyloxyethoxyphenyl, p-cyclopropyloxy ethoxyphenyl, p-cyclopropylmethoxy ethoxyphenyl, p-ethoxyethoxyphenyl and p-(p-F-phenyl)ethoxyethoxyphenyl.

Preferably n1 and n2 are both zero and the compound is of formula I-0

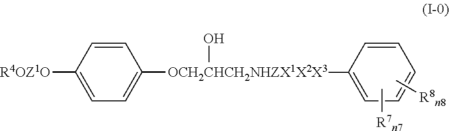

wherein all interegers are as hereinbefore defined.

Reference hereinbelow to formula I is taken as reference to formula I-0 and subformulae.
Preferably Z¹ is methylene, linear ethylene or linear propylene.
Preferably R⁴ is selected from C₃-C₇ cycloalkyl such as C₃ and C₅ cycloalkyl, unsubstituted and substituted C₁-C₃ linear alkyl, C₆₋₁₀ aryl, and combinations thereof, wherein substituents are as hereinbefore defined for R¹ and R²;
more preferably R⁴ is selected from unsubstituted and substituted C₃₋₇ cycloalkyl, C₁₋₃ alkyl, C₃₋₇ cycloalkyl-C₀₋₃ alkyl, C₆₋₁₀ aryl-C₀₋₃ alkyl, C₁₋₆alkoxy-C₆₋₁₀aryl-C₀₋₃ alkyl;
most preferably R⁴ is selected from cyclopentyl, cyclopropyl, c.prCH₂, Ph, CH₃OPhCH₂, PhCH₂CH₂, wherein Ph is optionally substituted by F, Cl or OH.
Preferably X¹ and X³ are as hereinbefore defined or X³ is a single bond.
Preferably X² is selected from CO, CS and SO₂.
Preferably n⁷ is 0, 1 or 2 and n⁸ is 0 or 1 and the sum thereof is 0, 1 or 2, more preferably at least one of n⁷ and n⁸ is 0;
Preferably R⁷ and R⁸ are selected from R⁴OZ¹O as hereinbefore defined, m-,p-(OCH₃)₂ and from o-, m- or p-OH, F, Cl, Br, NH₂, NO₂, OC₁₋₂ alkyl optionally substituted by halo or phenyl, COOH, COOC₁₋₂alkyl, CONH₂, CF₃, C₁₋₂ alkyl, phenyl and a combination thereof.

Preferably compounds of formula I do not include an imageable entity selected from ¹⁸F, ⁷⁶Br, ¹²⁴⁻⁵I, ¹³¹I, metal chelator or metal chelate complex for an MRI, a ligand for the complexation of a metal for SPECT, a lipid for incorporation into a liposome or the lipid itself. Preferably a compound as hereinbefore or hereinbelow defined does not include an imageable entity selected from ¹⁸F, ⁷⁶Br, ¹²⁴⁻⁵I, ¹³¹I, metal chelator or metal chelate complex for an MRI, a ligand for the complexation of a metal for SPECT, a lipid for incorporation into a liposome or the lipid itself.

More preferably there is provided a compound of formula IA and its pharmaceutically acceptable salt or salts and physiologically hydrolysable derivatives:

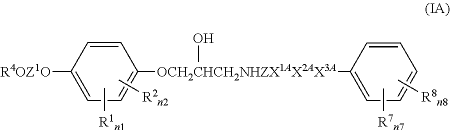

wherein
all integers are as hereinbefore defined; and
X^{1A} is selected from O and NH;
X^{2A} is selected from unsaturated C and unsaturated S; and
X^{3A} is selected from NH and CH₂; or
one of X^{1A}, X^{2A} and X^{3A} is a single bond;
   with the proviso that it is not a compound as listed in Tables A1, A2 and A5 above.

In one preferred selection embodiment there is provided a compound of formula Ia and its pharmaceutically acceptable salt or salts and physiologically hydrolysable derivatives:

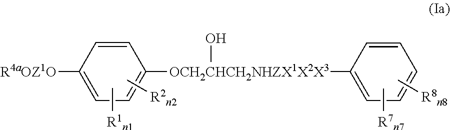

wherein
all integers are as hereinbefore defined; and
R^{4a} is optionally substituted C₃-C₈ cycloalkyl; more preferably is cyclopentyl
with the proviso that it is not a compound as shown in the following table:

TABLE A4^{a}

| R^{4a} | Z¹ | R¹ₙ₁, R²ₙ₂ | Z¹ | Z | X¹ | X² | X³ | R⁷ₙ₇, R⁸ₙ₈ |
|---|---|---|---|---|---|---|---|---|
| CH₃c.pr | CH₂CH₂ | o-CN | CH₂CH₂ | CH₂CH₂ | O | — | — | p-OCH₃ |

In a further preferred selection embodiment there is provided a compound of formula Ib and its pharmaceutically acceptable salt or salts and physiologically hydrolysable derivatives:

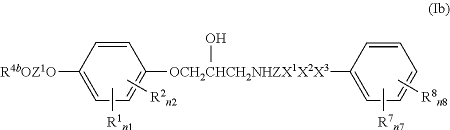

wherein
all integers are as hereinbefore defined; and
R^{4b} is C₁-C₄ linear or branched alkyl-C₃-C₈ cycloalkyl; preferably is cyclopropylmethyl, more preferably remaining integers are as defined for formula 1A
with the proviso that it is not a compound as shown in the following Tables A1 to A6 above:

In a further preferred selection embodiment there is provided a compound of formula Ic and its pharmaceutically acceptable salt or salts and physiologically hydrolysable derivatives:

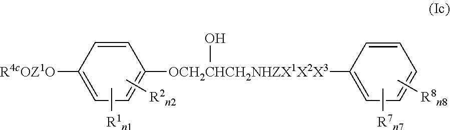

wherein
all integers are as hereinbefore defined; and
R^{4c} is ethyl.

In a further preferred selection embodiment there is provided a compound of formula Id and its pharmaceutically acceptable salt or salts and physiologically hydrolysable derivatives:

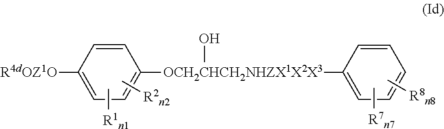

wherein
all integers are as hereinbefore defined; and
R^{4d} is optionally substituted C₅-C₁₀ aryl-C₀₋₃ alkyl; more preferably is PhCH₂CH₂; most preferably substituted by one or more R⁷ as hereinbefore defined, for example F.

In a further preferred selection embodiment there is provided a compound of formula Ie and its pharmaceutically acceptable salt or salts and physiologically hydrolysable derivatives:

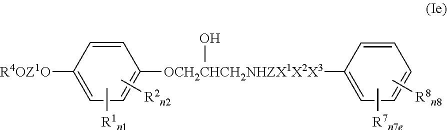

wherein
all integers are as hereinbefore defined; and
R^{7e} is p-OH and n7e is 1,
with the proviso that it is not a compound as shown in Tables A1 to A6 above:

In further embodiments, a compound of formula I is not a compound as listed in the following Tables A1B-A4B:

TABLE A1B

| | | R¹ₙ₁, | | | | | |
|---|---|---|---|---|---|---|---|
| R⁴ | Z¹ | R²ₙ₂ | Z | X¹ | X² | X³ | R⁷ₙ₇, R⁸ₙ₈ |
| C₁₋₅ alkyl | C₂₋₃ | — | C₂₋₅ | NH | CO | NH | — |
| C₃₋₅ alkenyl | alkylene | Mono | alkyl- | | | — | (C₁₋₄alk)₁₋₃ |
| C₅₋₇ cycloalkyl | | subst. | ene | | | CH₂ | (C₁₋₄alkoxy)₁₋₃ |
| C₃₋₇ cycloalkyl- | | | | | | | (CH₂OH)₁₋₂ |
| C₁₋₄-alkyl | | | | | | | (carbamoyl)₁₋₂ |
| o.s. Ph | | | | | | | (CN)₁₋₂ |
| o.s. C₇₋₁₀ | | | | | | | (Cl)₁₋₂ |
| Phalkyl | | | | | | | (Br)₁₋₂ |
| o.s. C₈₋₁₁Ph | | | | | | | F₁₋₂ |
| alkenyl | | | | | | | I₁₋₂ |
| where o.s. | | | | | | | (OH)₁₋₂ |
| is (C₁₋₄ | | | | | | | CF₃ |
| alky1)₁₋₃, | | | | | | | alkanoylamino |
| (C₁₋₄ alkoxy)₁₋₃ | | | | | | | C₁₋₄alkoxyC₁₋₄ |
| F₁₋₃ | | | | | | | alkoxy |
| Cl₁₋₃ | | | | | | | amino |
| Br₁₋₃ | | | | | | | alkanoylamino |
| | | | | | | | or |
| | | | | | | | combination |
| | | | | | | | thereof |

More preferably R⁴ is not alkyl, alkenyl, cycloalkyl, cycloalkylalkyl, o.s. aryl, o.s. aralkyl, o.s. aralkenyl and R⁷ₙ₇, R⁸ₙ₈ are not absent or any substituent;

TABLE A2B

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| Ph | CH₂CH₂ | — | CH₂CH₂ | NH | CO | NH | — |
| CH₃OPhCH₂ | | | | | | — | |
| FPh(CH₂)₁₋₂ | | | | | | | |
| Ph(CH₂)₂ | | | | | | | |
| c.prCH₂ | | | | | | | |
| c.prCH₂ | CH₂CH₂ | o-Br | CH₂CH₂ | NH | CO | NH | Subs. |
| i.pr | | o-CN | | | | — | |
| | | m-Br | | | | | |
| CH₃ | CH₂CH₂ | Disubst. | CH₂CH₂ | NH | CO | NH | Subs. |
| | | | | | | — | |
| CH₃ | CH₂CH₂ | — | CH₂CH₂ | NH | CO | NH | Unsubs. |
| | | | | | | — | Monosubs. |

TABLE A4B

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| hydrocarbon | C₂₋₃alkylene | — | C₂₋₅alkylene | O | — | — | — |
| | | o-Subst. | | S | | | (C₁₋₄alkyl)₁₋₃ |
| | | | | | | | (C₁₋₄alkoxy)₁₋₃ |
| | | | | | | | (halo)₁₋₃ |

For the avoidance of doubt, formula I and subformulae as hereinbefore defined do not include any of the following compounds:

TABLE A7
WO2008/083054

| | | | R¹ₙ₁, | | | | | R⁷ₙ₇, |
|---|---|---|---|---|---|---|---|---|
| | R⁴ | Z¹ | R²ₙ₂ | Z | X¹ | X² | X³ | R⁸ₙ₈ |
| XIV | CH₂CH₂F¹⁸ | CH₂CH₂ | — | CH₂CH₂ | NH | CO | NH | 5-tetra- |
| | | | | | | | | zole |
| VI | CH₂CH₂F¹⁸ | CH₂CH₂ | — | CH₂CH₂ | NH | CO | NH | 6-inda- |
| | | | | | | | | zole |

For the avoidance of doubt, R⁷ is not a heteroaromatic or heterocyclic moiety (EP400519, U.S. Pat. No. 5,135,932).

Preferably a compound of formula I is not as in the following:

TABLE A8
CAPLUS Registry compounds

| R⁴ | Z¹ | R¹ₙ₁, R²ₙ₂ | Z | X¹ | X² | X³ | R⁷ₙ₇, R⁸ₙ₈ |
|---|---|---|---|---|---|---|---|
| PhCH₂CH₂ | CH₂CH₂ | o-CN | CH₂CH₂ | NH | CO | NH | — |
| p-FPhCH₂CH₂ | | | | — | | | p-COOH |
| p-FPhCH₂ | | | | | | | |

Schemes 3-8 in the figures and Table 1 below present a sample of representative compounds of formula I and subformulae and their key precursors. The schemes and tables are illustrative only and are not intended to be exclusive:

TABLE 1

| Cpd | R⁴ | Z¹ | R¹ₙ₁, R²ₙ₂ | Z | X¹ | X² | X³ | R⁷ₙ₇, R⁸ₙ₈ |
|---|---|---|---|---|---|---|---|---|
| 45a | c.prCH₂ | CH₂CH₂ | — | CH₂CH₂ | NH | C(═O) | NH | p-OCH₂Ph |
| 45d | CH₃CH₂ | CH₂CH₂ | — | CH₂CH₂ | NH | C(═O) | NH | p-OCH₂Ph |
| 46a | c.prCH₂ | CH₂CH₂ | — | CH₂CH₂ | NH | C(═O) | NH | p-OH |
| 46b | p-FPh(CH₂)₂ | CH₂CH₂ | — | CH₂CH₂ | NH | C(═O) | NH | p-OH |
| 46d | CH₃CH₂ | CH₂CH₂ | — | CH₂CH₂ | NH | C(═O) | NH | p-OH |
| 46e | c.prCH₂ | CH₂CH₂ | — | CH₂CH₂ | NH | C(═O) | NH | m-Cl |
| 46f | CH₃CH₂ | CH₂CH₂ | — | CH₂CH₂ | NH | C(═O) | NH | m-Cl |
| 46g | CH₃CH₂ | CH₂CH₂ | — | CH₂CH₂ | NH | C(═O) | CH₂ | o-OH |
| 46h | CH₃CH₂ | CH₂CH₂ | — | CH₂CH₂ | NH | C(═O) | CH₂ | m-OH |
| 46i | c.prCH₂ | CH₂CH₂ | — | CH₂CH₂ | NH | C(═O) | CH₂ | p-OH |
| 46k | iso-propyl | CH₂CH₂ | — | CH₂CH₂ | NH | C(═O) | NH | m-Cl |
| 46l | iso-propyl | CH₂CH₂ | — | CH₂CH₂ | NH | C(═O) | NH | p-OH |
| 46m | n-propyl | CH₂CH₂ | — | CH₂CH₂ | NH | C(═O) | NH | m-Cl |
| 46n | n-propyl | CH₂CH₂ | — | CH₂CH₂ | NH | C(═O) | NH | p-OH |
| 46o | c.pr | CH₂CH₂ | — | CH₂CH₂ | NH | C(═O) | NH | m-Cl |
| 46p | c.pr | CH₂CH₂ | — | CH₂CH₂ | NH | C(═O) | NH | p-OH |
| 47a | c.pent | CH₂CH₂ | — | CH₂CH₂ | NH | C(═O) | NH | — |
| 47b | c.pent | CH₂CH₂ | — | CH₂CH₂ | NH | C(═O) | NH | o-CH₃ |
| 47c | c.pent | CH₂CH₂ | — | CH₂CH₂ | NH | C(═O) | NH | m-CH₃ |
| 47d | c.pent | CH₂CH₂ | — | CH₂CH₂ | NH | C(═O) | NH | p-CH₃ |
| 47e | c.pent | CH₂CH₂ | — | CH₂CH₂ | NH | C(═O) | NH | o-OCH₃ |
| 47f | c.pent | CH₂CH₂ | — | CH₂CH₂ | NH | C(═O) | NH | m-OCH₃ |
| 47g | c.pent | CH₂CH₂ | — | CH₂CH₂ | NH | C(═O) | NH | p-OCH₃ |
| 47h | c.pent | CH₂CH₂ | — | CH₂CH₂ | NH | C(═O) | NH | o-F |
| 47i | c.pent | CH₂CH₂ | — | CH₂CH₂ | NH | C(═O) | NH | m-F |
| 47j | c.pent | CH₂CH₂ | — | CH₂CH₂ | NH | C(═O) | NH | p-F |
| 47k | c.pent | CH₂CH₂ | — | CH₂CH₂ | NH | C(═O) | NH | o-Cl |
| (R)-47l | c.pent | CH₂CH₂ | — | CH₂CH₂ | NH | C(═O) | NH | m-Cl |
| (S)-47l | c.pent | CH₂CH₂ | — | CH₂CH₂ | NH | C(═O) | NH | m-Cl |
| 47m | c.pent | CH₂CH₂ | — | CH₂CH₂ | NH | C(═O) | NH | p-Cl |
| 47n | c.pent | CH₂CH₂ | — | CH₂CH₂ | NH | C(═O) | NH | o-Br |
| 47o | c.pent | CH₂CH₂ | — | CH₂CH₂ | NH | C(═O) | NH | m-Br |
| 47p | c.pent | CH₂CH₂ | — | CH₂CH₂ | NH | C(═O) | NH | p-Br |
| 47q | c.pent | CH₂CH₂ | — | CH₂CH₂ | NH | C(═O) | NH | o-CF₃ |
| 47r | c.pent | CH₂CH₂ | — | CH₂CH₂ | NH | C(═O) | NH | m-CF₃ |
| 47s | c.pent | CH₂CH₂ | — | CH₂CH₂ | NH | C(═O) | NH | p-CF₃ |
| 47t | c.pent | CH₂CH₂ | — | CH₂CH₂ | NH | C(═O) | NH | o-OH |
| 47u | c.pent | CH₂CH₂ | — | CH₂CH₂ | NH | C(═O) | NH | m-OH |
| 47v | c.pent | CH₂CH₂ | — | CH₂CH₂ | NH | C(═O) | NH | p-OH |
| 47w | c.pent | CH₂CH₂ | — | CH₂CH₂ | NH | C(═O) | NH | p-NO₂ |
| 47x | c.pent | CH₂CH₂ | — | CH₂CH₂ | NH | C(═O) | NH | p-OCH₂CH₂F |
| 47y | c.pent | CH₂CH₂ | — | CH₂CH₂ | NH | C(═O) | NH | m-(C═O)OMe |
| 47z | c.pent | CH₂CH₂ | — | CH₂CH₂ | NH | C(═O) | NH | p-(C═O)OMe |
| 47aa | c.pent | CH₂CH₂ | — | CH₂CH₂ | NH | C(═O) | NH | m-(C═O)OH |
| 47bb | c.pent | CH₂CH₂ | — | CH₂CH₂ | NH | C(═O) | NH | p-(C═O)OH |
| 48 | c.pent | CH₂CH₂ | — | CH₂CH₂ | NH | — | — | — |
| 49 | c.pent | CH₂CH₂ | — | CH₂CH₂ | NH | C(═O) | CH₂ | — |
| 50 | c.pent | CH₂CH₂ | — | CH₂CH₂CH₂ | — | C(═O) | NH | — |
| 51 | c.pent | CH₂CH₂ | — | CH₂CH₂ | O | C(═O) | NH | — |
| 52 | c.pent | CH₂CH₂ | — | CH₂CH₂ | NH | C(═O) | NH | — |
| 53 | c.pent | CH₂CH₂ | — | CH₂CH₂ | NH | S(═O)₂ | CH₂ | — |
| 54 | c.pent | CH₂CH₂ | — | CH₂CH₂ | NH | C(═O) | NH | m-F, p-OH |
| 54a | c.pent | CH₂CH₂ | — | CH₂CH₂ | NH | C(═O) | NH | m-F, p-F |
| 54b | c. pent | CH₂CH₂ | — | CH₂CH₂ | NH | C(═O) | NH | m-Cl, p-OMe |
| 54c | c.pent | CH₂CH₂ | — | CH₂CH₂ | NH | C(═O) | NH | m-Cl, p-OH |
| 54d | c.pent | CH₂CH₂ | — | CH₂CH₂ | NH | C(═O) | — | o-OH |
| 54e | c.pent | CH₂CH₂ | — | CH₂CH₂ | NH | C(═O) | — | m-OH |
| 54f | c.pent | CH₂CH₂ | — | CH₂CH₂ | NH | C(═O) | — | p-OH |
| 54g | c.pent | CH₂CH₂ | — | CH₂CH₂ | NH | C(═O) | — | o-F |
| 54h | c.pent | CH₂CH₂ | — | CH₂CH₂ | NH | C(═O) | — | m-F |
| 54i | c.pent | CH₂CH₂ | — | CH₂CH₂ | NH | C(═O) | — | p-F |
| 54j | c.pent | CH₂CH₂ | — | CH₂CH₂ | NH | C(═O) | CH₂ | m-OH |
| 60a | c.pent | CH₂CH₂ | — | CH₂CH₂ | NH | C(═O) | NH | o-NH₂ |
| 60b | c.pent | CH₂CH₂ | — | CH₂CH₂ | NH | C(═O) | NH | m-NH₂ |
| 60c | c.pent | CH₂CH₂ | — | CH₂CH₂ | NH | C(═O) | NH | p-NH₂ |
| 62a | c.pent | CH₂CH₂ | — | CH₂CH₂ | NH | C(═O) | — | — |
| 62b | c.pent | CH₂CH₂ | — | CH₂CH₂ | NH | C(═O) | — | o-NH₂ |
| 62c | c.pent | CH₂CH₂ | — | CH₂CH₂ | NH | C(═O) | — | m-NH₂ |
| 62d | c.pent | CH₂CH₂ | — | CH₂CH₂ | NH | C(═O) | — | p-NH₂ |
| 62e | c.pent | CH₂CH₂ | — | CH₂CH₂ | NH | C(═O) | — | p-OAc |
| 62f | c.pent | CH₂CH₂ | — | CH₂CH₂ | NH | C(═O) | — | m-OMe, p-OH |
| 62g | c.pent | CH₂CH₂ | — | CH₂CH₂ | NH | C(═O) | CH₂ | o-F |
| 62h | c.pent | CH₂CH₂ | — | CH₂CH₂ | NH | C(═O) | CH₂ | m-F |
| 62i | c.pent | CH₂CH₂ | — | CH₂CH₂ | NH | C(═O) | CH₂ | p-F |
| 62j | c.pent | CH₂CH₂ | — | CH₂CH₂ | NH | C(═O) | CH₂ | o-C1 |
| 62k | c.pent | CH₂CH₂ | — | CH₂CH₂ | NH | C(═O) | CH₂ | m-C1 |
| 62l | c.pent | CH₂CH₂ | — | CH₂CH₂ | NH | C(═O) | CH₂ | p-C1 |
| 62m | c.pent | CH₂CH₂ | — | CH₂CH₂ | NH | C(═O) | CH₂ | o-CF₃ |
| 62n | c.pent | CH₂CH₂ | — | CH₂CH₂ | NH | C(═O) | CH₂ | m-CF₃ |
| 62o | c.pent | CH₂CH₂ | — | CH₂CH₂ | NH | C(═O) | CH₂ | p-CF₃ |
| 62p | c.pent | CH₂CH₂ | — | CH₂CH₂ | NH | C(═O) | CH₂ | o-CH₃ |
| 62q | c.pent | CH₂CH₂ | — | CH₂CH₂ | NH | C(═O) | CH₂ | m-CH₃ |
| 62r | c.pent | CH₂CH₂ | — | CH₂CH₂ | NH | C(═O) | CH₂ | p-CH₃ |
| 62s | c.pent | CH₂CH₂ | — | CH₂CH₂ | NH | C(═O) | CH₂ | m-OMe |
| 62t | c.pent | CH₂CH₂ | — | CH₂CH₂ | NH | C(═O) | CH₂ | p-OMe |
| 62u | c.pent | CH₂CH₂ | — | CH₂CH₂ | NH | C(═O) | CH₂ | o-OH |
| 62v | c.pent | CH₂CH₂ | — | CH₂CH₂ | NH | C(═O) | CH₂ | p-OH |
| 62w | c.pent | CH₂CH₂ | — | CH₂CH₂ | NH | C(═O) | CH₂ | p-OAc |
| 65a | c.pent | CH₂CH₂ | — | CH₂CH₂ | O | — | — | m-Cl |
| 65c | c.pent | CH₂CH₂ | — | CH₂CH₂ | O | — | — | o-Ph |
| 65d | c.pent | CH₂CH₂ | — | CH₂CH₂ | O | — | — | p-Ph |
| 65e | c.pent | CH₂CH₂ | — | CH₂CH₂ | O | — | — | p-OH |
| 65f | c.pent | CH₂CH₂ | — | CH₂CH₂CH₂ | O | — | — | p-OH |
| 67a | c.pent | CH₂CH₂ | — | CH₂CH₂ | O | — | — | o-(C═O)NH₂ |
| 67b | c.pent | CH₂CH₂ | — | CH₂CH₂ | O | — | — | m-(C═O)NH₂ |
| 67c | c.pent | CH₂CH₂ | — | CH₂CH₂ | O | — | — | p-(C═O)NH₂ |
| 67d | c.pent | CH₂CH₂ | — | CH₂CH₂ | O | — | — | p-F |
| 67e | c.pent | CH₂CH₂ | — | CH₂CH₂ | O | — | — | p-OMe |
| 72 | c.pent | CH₂CH₂ | — | CH₂CH₂ | O | — | — | o-(C═O)NH₂, |
| | | | | | | | | p-OH |

A compound as hereinbefore defined may be in free form, i.e. normally as a base, or in any suitable salt or ester form. Free forms of the compound may be converted into salt or ester form and vice versa, in conventional manner. Suitable salts include hydrochloride, dihydrochloride, hydroformate, amide, succinate, half succinate, maleate, acetate, trifluoroacetate, fumarate, phthalate, tetraphthalate, benzoate, sulfonate, sulphate, phosphate, oxalate, malonate, hydrogen malonate, ascorbate, glycolate, lactate, malate, tartarate, citrate, aspartate or glutamate and variants thereof. Suitable acids for acid addition salt formation include the corresponding acids, i.e. hydrochloric, formic, amino acid, succinic, maleic, acetic, trifluoroacetic, fumaric, phthalic, tetraphthalic, benzoic, sulfonic, sulphuric, phosphoric, oxalic, malonic, ascorbic, glycolic, lactic, malic, tartaric, citric, aspartic or glutamic acids and the like.

Suitable esters include those obtained with the above acids, with hydroxides such as sodium, potassium, calcium or the like, or with alcohols.

The compounds of formula I and subformulae are optically active and may be prepared as one or both enantiomeric or tautomeric forms, or stereo or geometric isomeric forms, where relevant. Such forms may be identified and prepared or isolated by methods known in the art. Reference herein to compounds of formula I also encompasses reference to crystalline forms, polymorphs, hydrous and anhydrous forms and prodrugs thereof.

In a further aspect of the invention there is provided a process for the preparation of a compound of formula I or subformulae as hereinbefore defined comprising

contacting a compound of formula LIa

R⁴OZ¹OPhOCH₂oxirane (LIa)

with a compound of formula RIa where X is NH

HNHZX¹X²X³Ph or salt thereof (RIa)

or contacting a compound of formula LIb

R⁴OZ¹OPhOH (LIb)

with a compound of formula RIb where X is CH₂

oxirane-CH₂NHZX¹X²X³Ph (RIb)

or contacting a compound of formula LIc or LIc(pg)

R⁴OZ¹OPhOCH₂CH(OH)CH₂N(CH₂Ph)ZX¹X²OtBu (LIc)

with a compound of formula RIVa or RIVb

X²═NPh (RIVa)

### X² is C═O

LX²X³Ph (RIVb)

where L is OH, eg X² is C═O, X³ is CH₂
wherein Z, X¹, X² and X³ are as hereinbefore defined and pg is CH₂Ph protecting the propanolamine N.

In the process above and hereinbelow, reference to Ph is, where appropriate and where not shown, to R⁴OZ¹OPh or PhR⁷n⁷R⁸n⁸ where R⁷ and R⁸ are protected with a protecting group (pg) if appropriate.

Suitably LIa is prepared by reaction of LIb with epichlorohydrin.

Suitably LIb is prepared by reduction of LIIa

R⁴OZ¹OPhOCH₂Ph (LIIa).

Suitably LIIa is prepared by reaction of LIIIa with PhOCH₂Ph

R⁴OZ¹OH (LIIIa)

Suitably LIc is prepared by reaction of CI with LIa

(pg1)NHZX¹X²O(pg2) (CI)

where pg1 is CH₂Ph and pg2 is tBu.

Suitably CI is prepared by t-butoxy carbonylation of CII

(pg1)NHZX¹H (CII).

Suitably RIa is prepared by reaction of RIIa with acid

tBuOCONHZX¹X²X³Ph (RIIa)

or by reaction of RIIb with hydrazine monohydrate

dioxoisoindolineZX¹X²X³Ph (RIIb)

or by reaction of RIIc with RIVa

HNHZX¹H (RIIc)

(eg 1,2 ethanediamine, commercially available)

X²═NPhR⁷ (RIVa)

eg where X² is CO and R⁷ is o-tolyl or benzyloxy, forming X³ is NH or by interchange of RIa where R^{7,8} has one value to RIa where R^{7,8} has another value.

Suitable acid may be selected from any acid, for example TFA (trifluoroacetic acid), HCl or MeOH/HCl.

Interchange of RIa may be in the case that R^{7,8} is p-OH, interchanged from R^{7,8} is p-CH₂OH

Where X¹X²X³ is O, LIa is prepared by reaction of tBuOCONHZOH(RIIe) with R^{7,8}PhOH.

Suitably RIIa is prepared by reaction of a compound of formula RIIIa

tBuOCONHZX¹H (RIIIa)

eg where X¹ is NH
with a compound of formula RIVa-b

X²═NPh (RIVa)

eg X² is C═O or C═S, forming X³ is NH

LX²X³Ph (RIVb)

where L is Cl or OH, eg X² is C═O or SO₂, X³ is CH₂ or O
or by reaction of a compound of formula RIIIb

tBuOCONHZX₁X₂OH (RIIIb)

where X¹ is NH and X² is CO.
with a compound of formula RIVc

HX³Ph (RIVc)

eg where X³ is NH, RIVc is aniline (PhNH₂, commercially available) or analogues, where X³ is O, RIVc is phenol or nitrophenol.

Suitably RIIb is prepared by reaction of a compound of formula RIIIc

dioxoisoindolineZCOOH (RIIIc)

with a compound of formula RIVc above, eg where X³ is NH, RIVc is aminophenol NH₂PhOH (commercially available) or analogues,
or by reaction of a compound of formula RIIId

dioxoisoindolineZOH (RIIId)

with a compound of formula RIVa above, where X² is C═O above forming X³ is NH.

Suitably RIIIa is prepared by reaction of a compound of formula RIIc above with Boc₂O (di-tert-butyl dicarboxylate).

Suitably RIIIb is prepared by reaction of a compound of formula RVa

HNHZX¹X²OH (RVa)

with Boc₂O (di-tert-butyl dicarboxylate)

Suitably RIIIc or RIIId is prepared by reaction of a compound of formula RVb or RVc with phthalic anhydride

HNHZCOOH (RVb)

HNHZOH (RVc)

eg 2-amino ethanol

Suitably RIVc where X³ is NH is prepared by reduction of the corresponding RVIa NO₂Ph (RVIa).

Suitably RIIb is prepared by reaction of a compound of formula RIId where X is CH₂

CH₂═CHCH₂NHZX¹X²X³Ph (RIId)

with peroxyacid, eg peroxy benzoic acid.

Suitably RIId is prepared by reaction of a compound of formula RIIIe

CH₂═CHCH₂NHZCOOH (RIIIe)

with DPPA, diphenylphosphoryl azide, triethylamine and toluene

Suitably LIIIa is prepared by reaction of LIVa with base, eg pyridinium para toluene sulphonate and EtOH

R⁴OZ¹O-tetrahydro-2H-pyran (LIVa)

or by reduction of LIVb or LIVc

R⁴OCH₂COOH (LIVb)

cyclopentanone ethylene ketal (LIVc).

Suitably LIVa or b is prepared by reaction of LVa with tetrahydro-2H-pyran, eg 2-chloro ethoxytetrahydro-2H-pyran or with acetic acid, eg chloro acetic acid

R⁴OH (LVa).

Suitably a process is as hereinbefore defined or as hereinbelow illustrated in the drawings.

In a further aspect of the invention there is provided a novel intermediate as hereinbefore defined. Preferably a novel intermediate is of formula LIa, LIb, LIc, RIa, RIb, RIIa, RIIb, RIId, LIVa or LIVb as hereinbefore defined. Novel intermediates include compounds 41a, b, c, d, e, f, g; 61; 40a, b, c, d; 59; 11; 12; 13; 16a, b, c, d, e, f, g, h, l, j, k, l, m, n, o, p, q, r, s, t, u; 18, 18a, 18b, 18c; 20; 22; 22a, b, c, d, e, f, g, h, 22i; 26, 26a, b, c; 30a, b; 34; 38; 38a, b, c, d; 57; 58; 15a, b, c, d, e, f, g, h, l, j, k, l, m, n, o, p, q, r; 17; 19; 21; 25; 29a, b; 64a, c, d, e, f; 66a, b, c, d, e; 71; 33; 37; 8c and 8g; as hereinbelow defined.

In a further aspect of the invention there is provided a process as hereinbefore defined for the preparation of a novel intermediate as hereinbefore defined or as hereinbelow illustrated in the figures.

### Therapeutic Use

In a further aspect of the invention there is provided the use of a compound of formula I or subformulae as hereinbefore defined in the prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided the use of a compound of formula I or subformulae as hereinbefore defined in the manufacture of a medicament for prevention or treatment of a condition selected from ischaemic heart disease (also known as myocardial infarction or angina), hypertension and heart failure, restenosis and cardiomyopathy, more preferably with concomitant respiratory disease, in particular asthma or COPD.

In a further aspect of the invention there is provided a method of treating a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, a compound of formula I or subformulae or pharmaceutically acceptable salt thereof as hereinbefore defined in an amount sufficient to treat the condition.

The use of a compound of the invention in the manufacture of a medicament as hereinbefore defined includes the use of the compound directly, or in any stage of the manufacture of such a medicament, or in vitro in a screening programme to identify further agents for the prevention or treatment of the hereinbefore defined diseases or conditions.

A further aspect of the invention relates to the use of a compound of formula I or a pharmaceutically acceptable salt or solvate or physiologically hydrolysable, solubilising or immobilising derivative thereof, in an assay for identifying candidate compounds capable of treating one or more disorders or diseases as hereinbefore defined.

### Pharmaceutical Compositions

In a further aspect of the invention there is provided a composition comprising a therapeutically effective amount of a compound of formula I or subformulae or its pharmaceutically acceptable salt or physiologically hydrolysable derivative as hereinbefore defined in association with one or more pharmaceutical carriers, excipients or diluents. Suitable carriers, excipients or diluents may be selected having regard to the intended mode of administration and standard practice. The pharmaceutical compositions may be for human or animal usage in human and veterinary medicine, preferably for treatment of a condition, disease or disorder as hereinbefore defined

Examples of suitable carriers include lactose, starch, glucose, methyl cellulose, magnesium stearate, mannitol, sorbitol and the like.

A composition or compound of the invention is suitably for any desired mode of administration including oral, rectal, vaginal, parenteral, intramuscular, intraperitoneal, intraarterial, intrathecal, intrabronchial, subcutaneous, intradermal, intravenous, nasal, buccal or sublingual and the like. An indicated daily dosage is from about 1 mg to about 500 mg and compositions for oral administration generally contain from about 0.25 mg to about 250 mg of the compound together with solid or liquid carriers and diluents. A therapeutically effective amount is any amount from 0.1% to 99.9% w/w.

A composition for oral administration is suitably formulated as a compressed tablet, tablet, capsule, gel capsule, powder, solution, dispersion, suspension or the like. Such forms may be produced according to known methods and may include any suitable binder, lubricant, suspending agent, coating agent or solubilising agent or combinations thereof.

A composition for administration by means of injection is suitably formulated as a sterile solution or emulsion from a suitable solution or powder. Alternatively a composition may be in the form of suppositories, pessaries, suspensions, emulsions, lotions, creams, ointments, skin patches, gels, solgels, sprays, solutions or dusting powders.

A composition may include one or more additional active ingredients or may be administered together with compositions comprising other active ingredients for the same or different condition. An additional active ingredient is suitably selected from a diuretic, calcium channel antagonist, angiotensin converting enzyme (ACE) inhibitor, angiotensin receptor antagonist and the like.

In a further aspect of the invention there is provided the use of a compound of formula I or subformulae or a composition as hereinbefore defined in the prevention or treatment of a condition selected from ischaemic heart disease (also known as myocardial infarction or angina), hypertension and heart failure. In a particular advantage a compound or composition of the invention may be administered to a subject with, or used in the prevention or treatment of a subject suffering from one of the above conditions and from respiratory disease, in particular from asthma or COPD. In a further advantage a compound or composition of the invention may be administered to a subject with, or used in the prevention or treatment of a subject suffering from one of the above conditions and intolerant to a side effect associated with known beta blockers. In a further advantage a compound or composition of the invention has good oral bioavailability.

We have found that the compounds and compositions of the invention block beta-1 mediated responses but have substantially no affect on beta-2 mediated responses in a conscious animal. The beta-1 mediated responses include tachycardia, reflex heart rate response etc and the like, and are implicated in the above conditions. The beta-2 mediated responses include peripheral vascular conductance, hypotension and the like and are implicated in respiratory conditions.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Experimental—Abbreviations 1°, primary; 4°, quaternary; Ar, aromatic ring; Bn, benzyl; BnBr, benzyl bromide; Boc, Cert-butylcarbonate; Boc₂O, di-tert-butyl dicarboxylate; br, broad; brine, saturated sodium chloride solution; C, carbon; cAMP, cyclic adenosine monophosphate; CDCl₃, deuterated chloroform; m-CPBA, meta-chloroperoxybenzoic acid; COMFA, comparative molecular field analysis; COSY, correlation spectroscopy; d, doublet; O₂O, deuterated water; DBAD, di-tert-butyl azodicarboxylate; DCC, d icyclohexylcarbodiimide; DCM, dichloromethane; dd, doublet of doublets; DEAD, diethyl azodicarboxylate; def, deformation; DEPT, distortionless enhanced polarisation transfer; DIAD, diisopropyl azodicarboxylate; DMF, N,N-dimethylformamide; DMSO, dimethyl sulphoxide; DMSO-d₆, deuterated dimethyl sulphoxide; DPPA, Diphenylphosphoryl azide; dt, doublet of triplets; EDC, 1-ethyl-3-(3-dimethylaminopropyl)carbodiimide hydrochloride; EDTA, ethylenediamine tetraacetic acid; eq, molar equivalents; ES, electrospray; Et₂O, diethyl ether; EtOAc, ethyl acetate; EtOH, ethanol; FA, formic acid; FT-IR, fourier transform—Infrared; H₂, hydrogen gas; HCl, hydrochloric acid; HFIP, 1,1,1,3,3,3-hexafluoropropan-2-ol; HMBC, heteronuclear multiple bond correlation; HPLC, high performance liquid chromatography; HSQC, heteronuclear single quantum correlation; J, Coupling constant; J_{CF}, Carbon-Fluorine coupling constant; K₂CO₃, Potassium carbonate; KHSO₄, potassium hydrogen sulfonate; KMnO₄, potassium permanganate; lit, literature; m, multiplet; MeCN, acetonitrile; MeOH, methanol; MgSO₄, anhydrous magnesium sulphate; Mp, melting point/° C.; MS, mass spectrometry; MW, microwave; m/z, observed ion; NaH, sodium hydride; NaHCO₃, Sodium Hydrogen Carbonate; NaOH, sodium hydroxide; NH₃, Aqueous ammonia solution (35%); NMR, nuclear magnetic resonance spectroscopy; Pd, palladium; PDE, phosphodiesterase; PE, petroleum ether 40-60; phth, phthalimide; PLC, preparative layer chromatography; PMA, phosphomolybdic acid; ppm, parts per million; PPTS, pyridinium para-tolueunesulphonate; cyclopentyl; ^{c}Pr, cyclopropyl; p-TsCl, para-toluene sulfonylchloride; q, quadruplet; Rt, retention time; rt, room temperature; s, singlet; str, stretch; t, triplet; TBME, tert-butyl methyl ether; TEA, triethylamine; TFA, trifluoroacetic acid; THF, tetrahydrofuran; THP, tetrahydropyran; TMS, tetramethylsilane; TOF, time of flight.

### General Chemistry

Chemicals and solvents were purchased from standard suppliers and used without further purification. Merck Kieselgel 60, 230-400 mesh, for flash column chromatography was supplied by Merck Kga (Darmstadt, Germany) and deuterated solvents were purchased from Goss International Limited (England) and Sigma-Aldrich Company Ltd (England).

Unless otherwise stated, reactions were carried out at ambient temperature. Reactions were monitored by thin layer chromatography on commercially available precoated aluminium backed plates (Merck Kieselgel 60 F₂₅₄). Visualisation was by examination under UV light (254 and 366 nm). General staining carried out with Ninhydrin, KMnO₄ or PMA. All organic extracts after aqueous work-up procedures were dried over MgSO₄ before gravity filtering and evaporation to dryness. Organic solvents were evaporated under reduced pressure at 40° C. (water bath temperature). Purification using preparative layer chromatography was carried out using Fluke silica gel 60 PF₂₅₄ containing gypsum (200 mm×200 mm×1 mm). Flash chromatography was performed using Merck Kieselgel 60 (0.040-0.063 mm).

Melting points were recorded on a Reichert 7905 apparatus, Mettler Toledo Melting Point System MP50, or Perkin Elmer Pyris 1 differential scanning calorimeter and were uncorrected. FT-IR spectra were recorded as thin films or KBr discs in the range of 4000-500 cm⁻¹ using and Avatar 360 Nicolet FT-IR spectrophotometer. Optical rotation was measured on a Bellingham-Stanley ADP220 polarimeter.

Mass spectra (TOF ES +/−) were recorded on a Waters 2795 separation module/micromass LCT platform.

¹H NMR spectra were recorded on a Bruker-AV 400 at 400.13 MHz. ¹³C NMR spectra were recorded at 101.62 MHz. Chemical shifts (δ) are recorded in ppm with reference to the chemical shift of the deuterated solvent/an internal TMS standard. Coupling constants (J) are recorded in Hz and the significant multiplicites described by singlet (s), doublet (d), triplet (t), quadruplet (q), broad (br), multiplet (m), doublet of doublets (dd), doublet of triplets (dt). Spectra were assigned using appropriate COSY, DEPT, HSQC and HMBC sequences. Unless otherwise stated all spectra were recorded in CDCl₃.

Analytical HPLC to confirm purity was performed using two different conditions from the following list. All retention times are quoted in minutes.

System 1 (s1): Phenomenex Onyx Monolithic reverse phase C₁₈ column (100×4.6 mm), a flow rate of 5.00 mL/min (system 1a) or 3.00 mL/min (system 1b) and UV detection at 287 nm. Linear gradient 5%-95% solvent B over 10 minutes. Solvent A: 0.1% FA in water; solvent B: 0.1% FA in MeCN.

System 2 (s2): Vydac reverse phase C₈ column (150×4.6 mm), a flow rate of 1.00 mL/min and UV detection at 287 nm. Linear gradient 5%-95% solvent B over 24 minutes. Solvent A: 0.06% TFA in water; solvent B: 0.06% TFA in MeCN.

System 3 (s3): Waters symmetry reverse phase C₁₈ column (75×4.6 mm), a flow rate of 1.00 mL/min and UV detection at 287 nm. Linear gradient 5%-95% solvent B over 20 minutes. Solvent A: 0.1% FA in water; solvent B: 0.1% FA in MeOH.

System 4 (s4): Shimadzu UFLCXR system coupled to an Applied Biosystems API2000. Gemini-NX 3u-110A, 50×2 mm column thermoregulated at 40° C. Flow rate 0.5 ml/min. UV detection at 220 and 254 nm. Gradient: Pre-equilibration run for one minute at 10% solvent B, 10 to 98% solvent B in 2 minutes, 98% solvent B for 2 minutes, 98 to 10% solvent B in 0.5 minutes, then 10% solvent B for one minute. Solvent A: 0.1% Formic Acid in water; Solvent B: 0.1% Formic Acid in MeCN.

System 5: Shimadzu UFLCXR system coupled to an Applied Biosystems API2000. Luna 3u (PFP2) 110A, 50×2 mm column thermoregulated at 40° C. Flow rate 0.5 ml/min. UV detection at 220 and 254 nm. Gradient: Pre-equilibration run for one minute at 10% solvent B, 10 to 98% solvent B in 2 minutes, 98% solvent B for 2 minutes, 98 to 10% solvent B in 0.5 minutes, then 10% solvent B for one minute. Solvent A: 0.1% Formic Acid in water; Solvent B: 0.1% Formic Acid in MeCN.

Preparative HPLC was performed using a Phenomenex Onyx Monolithic reverse phase C₁₈ column (100×10 mm), a flow rate of 14.10 mL/min and UV detection at 287 nm. Samples were run in 5%-95% solvent B over 10 minutes. Solvent A: 0.1% FA in water; solvent B: 0.1% FA in MeCN.

### Preparation of Intermediates

### Scheme 1 and 2

### 2-(2-(Cyclopropylmethoxy)ethoxy)-tetrahydro-2H-pyran (2)

NaH 60% suspension in mineral oil (6.659 g, equivalent to 3.995 g of NaH, 0.166 mol, 1.2 eq) was weighed into a flame-dried flask and washed with hexanes (2×50 mL) under nitrogen atmosphere. Residual hexanes were allowed to evaporate under nitrogen flow before suspending the NaH in dry THF and cooling to 0° C. 1 (10.000 g, 0.139 mol) was dissolved in dry THF (20 mL) and dry DMF (30 mL) before adding dropwise over 30 minutes to the suspended NaH with stirring. The mixture was brought to rt before dropwise addition of 2-chloroethoxytetrahydro-2H-pyran (30.71 mL 0.208 mol 1.5 eq) in dry THF (20 mL) over 30 minutes. The mixture was stirred at rt overnight before quenching with MeOH (20 mL). All solvents were removed before dissolving the residue in Et₂O (200 mL) and washing with water (2×150 mL) and brine (150 mL). After removal of solvent, the resulting crude oil was purified by flash column chromatography (eluent DCM) to give 5.878 g colourless oil.

### 2-(Cyclopropylmethoxy)ethanol (3)

2 (1.800 g, 8.99 mmol) was diluted in EtOH (60 mL). PPTS (226 mg, 0.90 mmol, 0.1 eq) in EtOH (15 mL) was added and the solution stirred at 55° C. for 4 h. Excess solvent was removed and on dilution of the residue with pet ether 40°-60° C./Et₂O (15:85), PPTS precipitated out. Following filtration of PPTS the remaining crude product was purified by flash column chromatography (eluent pet ether 40°-60° C./Et₂O 15:85) to afford 670 mg colourless oil.

### 2-(4-Fluorophenethyloxy)acetic acid (5)

NaH 60% suspension in mineral oil (2.400 g, equivalent to 1.440 g of NaH, 60 mmol, 2 eq) was weighed into a flame-dried flask and suspended in dry DMF (60 mL) with stirring, under a nitrogen atmosphere. To this was added 4 (4.205 g, 3.751 mL, 30 mL) and the temperature raised to 60° C. with stirring for 15 minutes. Chloroacetic acid (2.835 g, 30 mmol, 1 eq) was added to the flask and the mixture allowed to stir at 60° C. for a further 2.5 h. After cooling and removal of solvent, the residue was suspended in Et₂O (30 mL) and extracted with water (2×30 mL). The combined aqueous layers were acidified with aqueous 2 M HCl (to around pH 3) before extraction with EtOAc (3×30 mL). After removal of solvent, the crude solid was recrystallised from cyclohexane to yield 3.000 g of pink crystals.

### 2-(4-Fluorophenethyloxy)ethanol (6)

Lithium Aluminium Hydride (472 mg, 12.45 mmol, 1 eq) suspended in anhydrous THF (15 mL) over ice with stirring. 5 (2.467 g, 12.45 mmol) in anhydrous THF (15 mL) was slowly dripped in the suspension over 10 minutes and the resulting mixture stirred overnight at rt under a nitrogen atmosphere. After quenching carefully with water, the suspension was filtered (gravity) and the filtrate concentrated to an oil. Purification was achieved by flash column chromatography (eluent EtOAc/Hexanes 60:40), yielding 1.52 g of clear, colourless oil.

### 2-(Cyclopentyloxy)ethanol (8)

Zirconium chloride (10.021 g, 43 mmol, 1.1 eq) was dissolved in dry THF (100 mL) under a nitrogen atmosphere. To this was added sodium borohydride (6.507 g, 172 mmol, 4.4 eq) in portions at rt with stirring, resulting in hydrogen gas evolution and formation of a cream suspension. A solution of 7 (5.000 g, 4.85 mL, 39 mmol) in dry THF (50 mL) was added slowly whilst maintaining the vessel temperature between 0-5° C. After stirring at rt for 4 h, the mixture was quenched with cautious addition of aqueous 2 M HCl over an ice bath. All organic solvent was removed under vacuum and the remaining aqueous slurry extracted with EtOAc (3×50 mL). The combined organic layers were washed with brine (1×30 mL) before concentration to a crude oil. This was purified by flash column chromatography (eluent EtOAc/hexanes 50:50) to give 4.114 g of clear colourless oil.

### 1-(2-(Allyloxy)ethoxy)-4-(benzyloxy)benzene (8b)

8a (10 g, 98 mmol), triphenylphosphine (30.8 g, 117 mmol, 1.2 eq), and 4-(benzyloxy)phenol (23.8 g, 117 mmol, 1.2 eq) were dissolved in DCM (350 mL). DIAD (23.14 mL, 117 mmol, 1.2 eq) in DCM (50 mL) was added dropwise to the reaction mixture and allowed to stir overnight. The mixture was concentrated to a slurry before redissolving in Et₂O (300 mL) and filtering any precipitated triphenylphosphine oxide. The filtrate was washed with aq. 2M NaOH (2×100 mL), water (100 mL) and brine (100 mL), before concentration to give an oily residue. This was further purified by FCC (eluent Et₂O/PE 10:90 for 2 column volumes, followed by 1:4 to elute) to give 25.8 g (93%) of 8b as a cream coloured solid.

### 1-(Benzyloxy)-4-(2-propoxyethoxy)benzene (8c)

8b (25.8 g, 90.73 mmol) was dissolved in anhydrous DCM (400 mL) under an atmosphere of nitrogen. Diethylzinc (91 mL, 90.73 mmol, 1.1 eq) was added, followed by CH₂I₂ (8.77 mL, 108.88 mmol, 1.2 eq). The mixture was stirred at rt overnight. Further additions of Simmons-Smith reagents did not cause the reaction to proceed to toal completion. After 10 days of stirring, the reaction mixture was poured on to aq. Sat NH₄Cl (200 mL) in ice, before extracting with DCM (3×150 mL). Initial shaking caused an emulsion, which was separated by passing through a bed of celite. The combined organic layers were then washed aq. Sat NaHCO₃ (100 mL). After drying with Na₂SO₄, the combined organic layers were passed once more through a bed of celite, followed by a plug of celite. After concentration, 24.6 g of a yellow crystalline solid was obtained. ¹H-nmr analysis indicated between 5-10% starting material was still present, with the remainder being the cyclopropanated product. The crude product was dissolved in THF (400 mL), before addition of 10% Pd/C (2.5 g) and hydrogenation at rt. After overnight stirring, no change from the starting material was noted by TLC analysis, so conc. HCl (5 mL) was added and hydrogenation continued for a further 10 days. The mixture was then filtered through a bed of celite, to give a brown crystalline solid. This was redissolved in DCM (100 mL) and washed with aq. sat. EDTA/sat. NaHCO₃(1:1) (100 mL). The aqueous layer was washed with DCM (2×200 mL). The combined organic layers were filtered through a bed of celite and concentrated to give a clear colourless oil, purified by FCC (eluent TBME/PE 0:100 to 30:70) to give 8c as white crystalline solid (1.569 g, 6%) and 39a (19.017 g, 70%).

### 4-(2-(Vinyloxy)ethoxy)phenyl benzoate (8e)

8d and 4-hydroxyphenyl benzoate underwent Mitsunobu coupling in a similar manner to that described for the synthesis of 8b.

### 4-(2-Cyclopropoxyethoxy)phenyl benzoate (8f)

Cyclopropanation of 8e was adapted from S.E.Denmark, J. P. Edwards, JOC, 1991, 56, 6974-6981.

### 4-(2-Cyclopropoxyethoxy)phenol (8g)

Saponification of 8f was carried out in 1,2-dioxane/water using NaOH, according to standard textbook protocol.

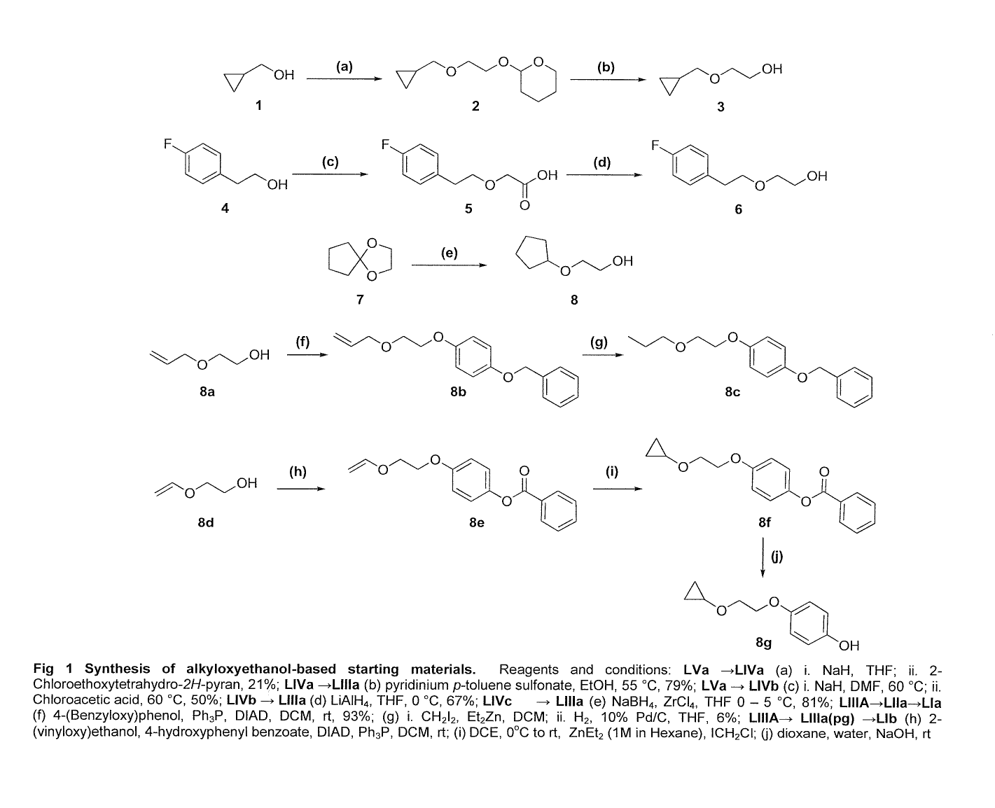

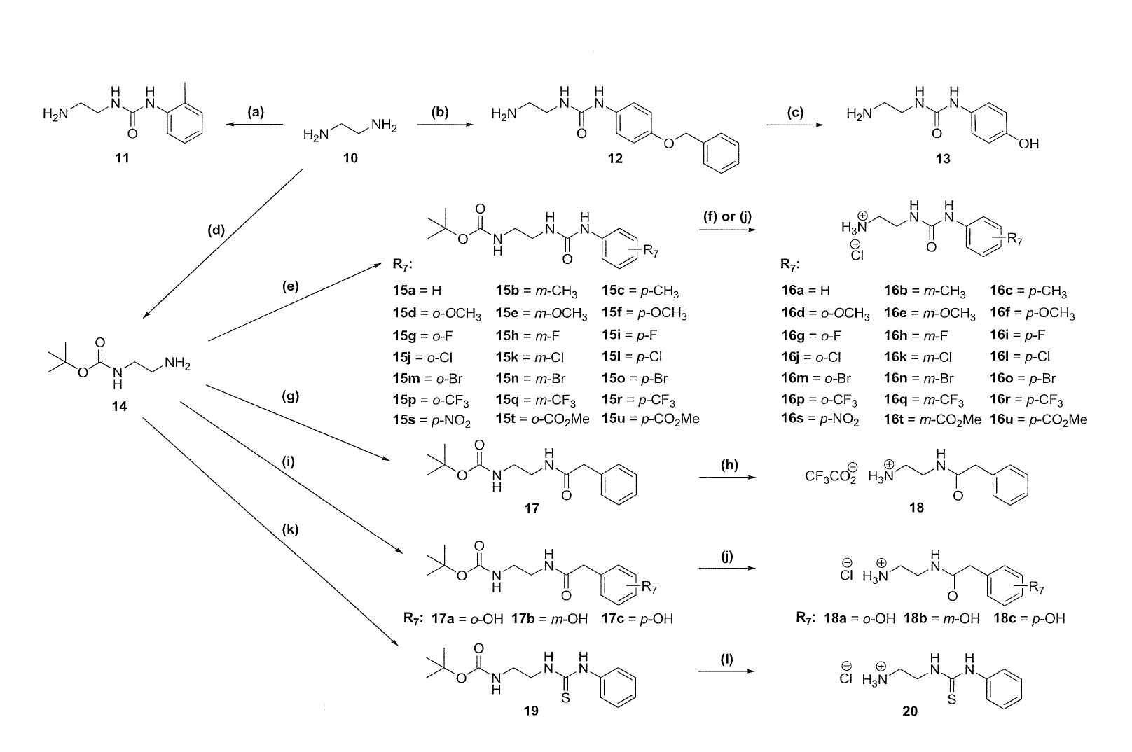

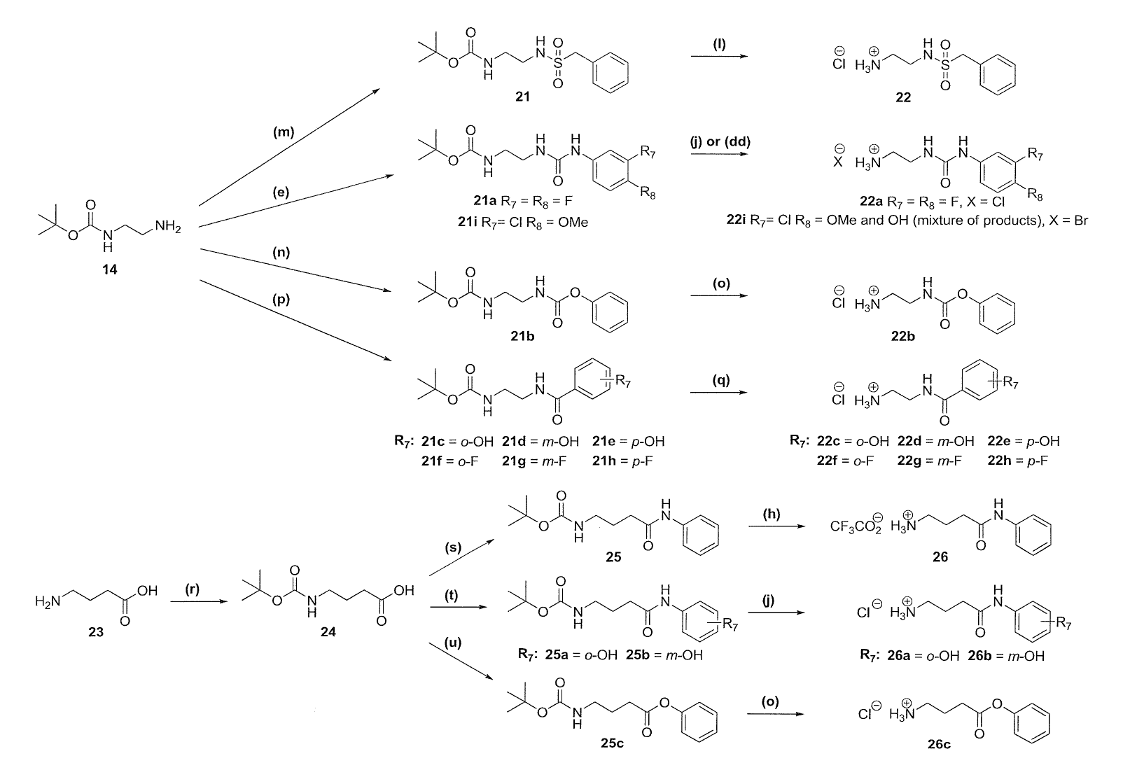

Table 2 lists the ¹H NMR spectral data for selected compounds from Figure 1:

| Cpd | m.p | ¹H NMR |
|---|---|---|
| 2 | δ 98.88 (CH THP group), 75.92 (OCH₂^{c}Pr), 69.65 (CH₂OCH₂^{c}Pr), 66.66 (THPOCH₂), 62.16 | |
| | (OCH₂ THP group), 30.53 (OCHCH₂ THP group), 25.41 (OCH₂CH₂ THP group), 19.46 (OCH | |
| | CH₂CH₂ THP group), 10.54 (^{c}Pr CH), 3.00, 2.91 (^{c}Pr CH₂). | |
| 3 | δ 3.73-3.77 (m, 2H, CH₂OH), 3.58 (t, J = 4.9 Hz, 2H, CH₂CH₂OH), 3.33 (d, J = 7.2 Hz, 2H, | |
| | ^{c}PrCH₂O), 2.06 (t, J = 6.6 Hz, 1H, OH), 1.08 (m, 1H, CH), 0.53-0.58 (m, 2H, ^{c}Pr CH₂)*, | |
| | 0.20-0.24 (m, 2H, ^{c}Pr CH₂)*. *Refers to cis-protons of ^{c}Pr ring. | |
| 5 | 84-86 | δ 8.5-10.4 (br s, 1H, CO₂H), 7.19 (dd, J = 8.6/5.7 Hz, 2H, aryl 3-H and 5-H), 6.99 |
| | | (dd, J = 8.6/8.6 Hz, 2H, aryl 2-H and 6-H), 4.12 (s, 2H, CH₂CO₂H), 3.76 (t, J = 6.8 Hz, |
| | | 2H, CH₂O), 2.92 (t, J = 6.8 Hz, 2H, ArCH₂). |

| | |
|---|---|
| 6 | δ 7.15 (dd, J = 8.6/5.5 Hz, 2H, aryl 3-H and 5-H), 6.95 (dd, J = 8.8/8.8 Hz, 2H, aryl 2-H and |
| | 6-H), 3.65-3.72 (m, 2H, CH₂OH), 3.65 (t, J = 7.0 Hz, 2H, ArCH₂CH₂), 3.53 (t, J = 4.8 Hz, |
| | 2H, OCH₂CH₂OH), 2.85 (t, J = 7.0 Hz, 2H, ArCH₂), 2.38 (br s, 1H, OH). |
| 8 | δ 3.84-3.89 (m, 1H, CH), 3.60-3.63 (m, CH₂OH), 3.41 (t, J = 4.9 Hz, 2H, ^{c}PeOCH₂), 2.84 |
| | (t, J = 5.6 Hz, 1H, OH), 1.51-1.72 (m, 6H, ^{c}Pe CH₂), 1.38-1.50 (m, 2H, ^{c}Pe CH₂). |

### 1-(2-Aminoethyl)-3-(4-(benzyloxy)phenyl)urea (12)

A solution of 4-(benzyloxy)phenylisocyanate (3.739 g 16.61 mmol) in anhydrous DCM (30 mL) was dripped into a flask containing vigorously stirred 10 (6 mL, 89.80 mmol, 5.4 eq) under nitrogen. Instant precipitation of a white solid was noted and the reaction was allowed to stir for a further 3 h. After removal of all volatiles, the crude solid was washed with Et₂O, before drying to give 4.472 g of white solid.

### 1-(2-Aminoethyl)-3-(4-hydroxyphenyl)urea (13)

12 (113 mg, 0.40 mmol) was stirred overnight in a solution of concentrated HCl (10 mL). All solvent was removed under vacuum and the residue redissolved in water (10 mL) before neutralisation with 0.5 M aqueous NaOH. After removal of water under reduced pressure, the residue was dissolved in the minimum amount of MeOH and filtered (gravity) before purification by PLC (eluent NH₃/MeOH/DCM 2:25:73). This gave 56 mg of brown semi-solid.

### tert-Butyl 2-aminoethylcarbamate (14)

10 (50 mL, 927 mmol, 8.75 eq) was diluted in DCM (200 mL) with vigorous stirring. Di-tert-butyl dicarbonate (23.2 g, 106 mmol) was dissolved in DCM (1.3 L) and then added dropwise to the solution of 1,2-ethanediamine over 24 hours. After removal of all volatiles, the remaining residue was partitioned between water (250 mL) and DCM (250 mL). The aqueous layer was washed again with DCM (250 mL) before combining the organic solvents and concentrating. The residue was dissolved in aqueous 0.5 M KHSO₄ (250 mL) and washed with DCM (2×100 mL). The aqueous layer was then basified with aqueous 2 M NaOH before final extraction with DCM (4×100 mL). The combined organic extracts were dried and concentrated to give 12.98 g of viscous translucent oil.

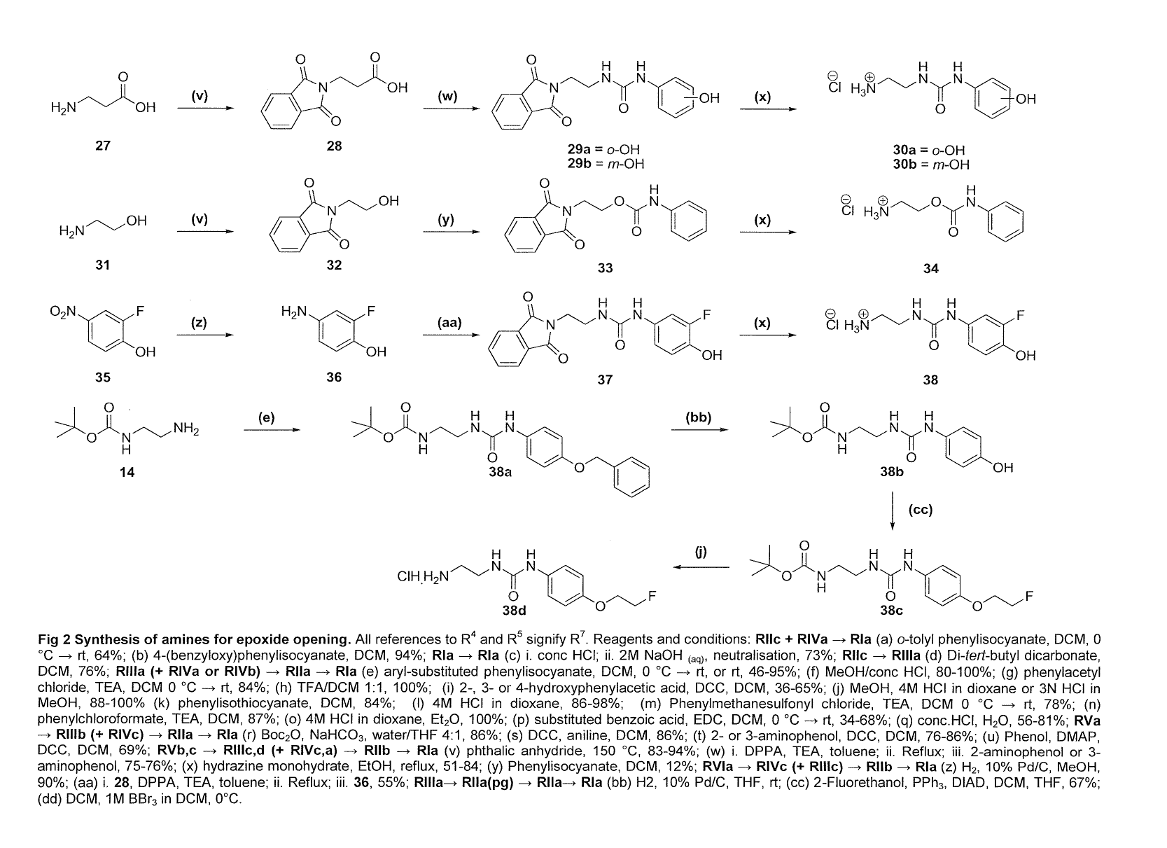

Table 3 lists the ¹H NMR spectral data for selected compounds from Figure 2:

| Cpd | m.p | ¹H NMR |
|---|---|---|
| 11 | 179-181 | (DMSO-d₆): −δ 7.81 (d, J = 8.1 Hz, 1H, aryl 6-H), 7.70 (br s, 1H, NH(C═O)NHAr), 7.05- |
| | | 7.11 (m, 2H, aromatic C—H), 6.85 (dd, J = 8.1/8.1 Hz, 1H, aromatic C—H), 6.64 (t, J = |
| | | 5.4 Hz, 1H, NH(C═O)NHAr), 3.07 (dt, J = 6.0/6.0 Hz, 2H, CH₂NH), 2.61 (t, J = 6.2 Hz, |
| | | 2H, CH₂NH₂), 2.17 (s, 3H, CH₃). |
| 12 | 147-149 | (DMSO-d₆): −δ 8.48 (s, 1H, NHAr), 7.31-7.44 (m, 5H, aromatic benzyl CH), 7.28, 6.88 |
| | | (d, J = 9.0 Hz, 2 x 2H, para-disubstituted ring), 6.24 (t, J = 5.2 Hz, 1H, NHCONHAr), |
| | | 5.02 (s, 2H, PhCH₂O), 4.27 (br s, 2H, NH₂), 3.10-3.17 (m, 2H, CH₂NH), 2.67 (t, J = |
| | | 6 Hz, CH₂NH₂) |

| | |
|---|---|
| 13 | (MeOD-d₄): δ 7.17 (d, J = 8.7 Hz, 2H, aryl 3-H and 5-H), 6.73 (d, J = 8.7 Hz, 2H, aryl 2-H and |
| | 6-H), 3.45 (t, J = 5.6 Hz, 2H, CH₂NH(C═O)NH), 3.05 (t, J = 6.0 Hz, 2H, CH₂NH₂). |
| 14 | δ 5.16 (br s, 1H, CONH), 3.06-3.10 (m, 2H, CH₂NH), 2.71 (t, J = 6 Hz, CH₂NH₂), 1.36 (s, |
| | 9H, C(CH₃)₃), 1.24 (br s, 2H, NH₂). |

### General Procedure for Synthesis of Phenyl Substituted tert-butyl 2-(3-phenylureido)ethylcarbamates

14 (1 eq) was dissolved in dry DCM (10 mL per 500 mg) and cooled to 0° C. with stirring under a nitrogen atmosphere. To this was added dropwise, a solution of the desired substituted phenylisocyanate (500 mg) in dry DCM (5 mL). In the case of 4-nitrophenylisocyanate, the amine solution was not cooled prior the isocyanate addition. The mixture was stirred overnight at rt, before addition of hexanes or petroleum ether 40-60, until precipitation occurred. The solid mass was collected by filtration (vacuum) and washed with hexanes before drying in vacuo to give the following compounds:

Table 4 lists the ¹H NMR spectral data for selected compounds from Figure 2:

| Cpd | m.p | ¹H NMR (DMSO-d₆) |
|---|---|---|
| 15a | 153-155 | δ 8.53 (s, 1H, NH(C═O)NHPh), 7.37 (d, J = 7.7 Hz, 2H, 2-H and 6-H phenyl ring), |
| | | 7.20 (dd, J = 7.5/7.5 Hz, 2H, 3-H and 5-H phenyl ring), 6.86-6.89 (m, 2H, 4-H |
| | | phenyl ring, NH(C═O)NHPh), 6.16 (t, J = 5.5 Hz, 1H, O(C═O)NH), 3.11 (dt, J = |
| | | 6.1/6.1 Hz, 2H, CH₂NH(C═O)NH), 2.99 (dt, J = 5.8/5.8 Hz, 2H, CH₂NH(C═O)O), |
| | | 1.38 (s, 9H, C(CH₃)₃). |
| 15b | 135-137 | δ 8.43 (s, 1H, NH(C═O)NHAr), 7.21 (s, 1H, aryl 2-H), 7.15 (d, J = 8.4 Hz, 1H, aryl 6- |
| | | H), 7.08 (dd, J = 7.6/7.6 Hz, 1H, aryl 5-H), 6.86 (t, J = 5.2 Hz, 1H, NH(C═O)NHAr), |
| | | 6.69 (d, J = 7.3 Hz, 1H, aryl 4-H), 6.12 (t, J = 5.5 Hz, 1H, O(C═O)NH), 3.11 (dt, J = |
| | | 6.4/6.4 Hz, 2H, CH₂NH(C═O)NH), 2.99 (dt, J = 5.8/5.8 Hz, 2H, CH₂NH(C═O)O), |
| | | 2.23 (s, 3H, CH₃), 1.37 (s, 9H, C(CH₃)₃). |
| 15c | 145-147 | δ 8.39 (s, 1H, NH(C═O)NHAr), 7.25 (d, J = 8.4 Hz, 2H, aryl 2-H and 6-H) 7.01 (d, J = |
| | | 8.3 Hz, 2H, aryl 3-H and 5-H), 6.85 (t, J = 5.1 Hz, 1H, NH(C═O)NHAr), 6.09 (t, J = |
| | | 5.5 Hz, 1H, O(C═O)NH), 3.10 (dt, J = 6.0/6.0 Hz, 2H, CH₂NH(C═O)NH), 2.98 (dt, J = |
| | | 5.8/5.8 Hz, 2H, CH₂NH(C═O)O), 2.20 (s, 3H, CH₃), 1.37 (s, 9H, C(CH₃)₃). |
| 15d | 155-157 | δ 8.06 (dd, J = 7.5/2.0 Hz, 1H, aryl 6-H), 7.91 (s, 1H, NH(C═O)NHAr), 6.90-6.96 |
| | | (m, 2H, aromatic C—H, NH(C═O)NHAr), 6.80-6.88 (m, 3H, aromatic C—H, |
| | | O(C═O)NH), 3.82 (s, 3H, OCH₃), 3.10 (dt, J = 6.4/6.4 Hz, 2H, CH₂NH(C═O)NH), |
| | | 2.98 (dt, J = 5.9/5.9 Hz, 2H, CH₂NH(C═O)O), 1.37 (s, 9H, C(CH₃)₃). |
| 15e | 159-161 | δ 8.53 (s, 1H, NH(C═O)NHAr), 7.13 (dd, J = 2.2/2.2 Hz, 1H, aryl 2-H), 7.10 (dd, J = |
| | | 8.1/8.1 Hz, 1H, aryl 5-H), 6.83-6.87 (m, 2H, aryl 6-H, NH(C═O)NHAr), 6.46 (dd, J = |
| | | 8.1/2.0 Hz, 1H, aryl 4-H), 6.14 (t, J = 5.6 Hz, 1H, O(C═O)NH), 3.70 (s, 3H, OCH₃), |
| | | 3.11 (dt, J = 6.4/6.4 Hz, 2H, CH₂NH(C═O)NH), 2.99 (dt, J = 5.8/5.8 Hz, 2H, |
| | | CH₂NH(C═O)O), 1.37 (s, 9H, C(CH₃)₃). |
| 15f | 102-104 | δ 8.31 (s, 1H, NH(C═O)NHAr), 7.27 (d, J = 9.0 Hz, 2H, aryl 2-H and 6-H), 6.85 (t, J = |
| | | 4.6 Hz, 1H, NH(C═O)NHAr), 6.80 (d, J = 9.0 Hz, 2H, aryl 3-H and 5-H), 6.04 (t, J = |
| | | 5.6 Hz, 1H, O(C═O)NH) 3.68 (s, 3H, OCH₃), 3.10 (dt, J = 6.4/6.4 Hz, 2H, |
| | | CH₂NH(C═O)NH), 2.98 (dt, J = 5.8/5.8 Hz, 2H, CH₂NH(C═O)O), 1.37 (s, 9H, |
| | | C(CH₃)₃). |
| 15g | 181-183 | δ 8.32 (s, 1H, NH(C═O)NHAr), 8.11 (dd, J = 7.2 Hz, aryl 5-H), 7.16 (ddd, J = |
| | | 11.8/8.2/1.4 Hz, 1H, aryl 3-H), 7.06 (dd, J = 7.9/7.9 Hz, 1H, aryl 4-H), 6.85-6.93 |
| | | (m, 2H, NH(C═O)NHAr, aryl 6-H), 6.66 (t, J = 5.5 Hz, 1H, O(C═O)NH), 3.12 (dt, J = |
| | | 6.3/6.3 Hz, 2H, CH₂NH(C═O)NH), 2.99 (dt, J = 5.8/5.8 Hz, 2H, CH₂NH(C═O)O), |
| | | 1.37 (s, 9H, C(CH₃)₃). |
| 15h | 114-116 | δ 8.78 (s, 1H, NH(C═O)NHAr), 7.45 (ddd, J = 12.3/2.2/2.2 Hz, 1H, aryl 2-H), 7.22 |
| | | (ddd, J = 8.2/8.2/8.2 Hz, 1H, aryl 5-H), 7.01 (dd, J = 8.2/1.1 Hz, 1H, aryl 6-H), 6.86 |
| | | (t, J = 5.3 Hz, 1H, NH(C═O)NHAr), 6.68 (dd, J = 8.7/2.5 Hz, 1H, aryl 4-H), 6.22 (t, J = |
| | | 5.5 Hz, 1H, O(C═O)NH), 3.12 (dt, J = 6.4/6.44 Hz, 2H, CH₂NH(C═O)NH), 3.00 (dt, |
| | | J = 5.8/5.8 Hz, 2H, CH₂NH(C═O)O), 1.37 (s, 9H, C(CH₃)₃). |
| 15i | 152-154 | δ 8.56 (s, 1H, NH(C═O)NHAr), 7.38 (dd, J = 7.0/5.0 Hz, 2H, aryl 2-H and 6-H), 7.04 |
| | | (dd, J = 8.9/8.9 Hz, 2H, aryl 3-H and 5-H), 6.86 (t, J = 5.3 Hz, 1H, NH(C═O)NHAr), |
| | | 6.13 (t, J = 5.5 Hz, 1H, O(C═O)NH), 3.11 (dt, J = 6.4/6.44 Hz, 2H, CH₂NH(C═O)NH), |
| | | 2.99 (dt, J = 5.8/5.8 Hz, 2H, CH₂NH(C═O)O), 1.37 (s, 9H, C(CH₃)₃). |
| 15j | 167-169 | δ 8.14 (d, J = 8.2 Hz, 1H, aryl 6-H), 8.03 (s, 1H, NH(C═O)NHAr), 7.38 (dd, J = |
| | | 8.0/1.4 Hz, 1H, aryl 3-H), 7.23 (ddd, J = 7.8/7.8/1.4 Hz, 1H, aryl C—H), 7.07 (t, J = 5.3 |
| | | Hz, 1H, NH(C═O)NHAr), 6.94 (dd, J = 7.8/1.5 Hz, 1H, aryl C—H), 6.87 (t, J = 5.2 Hz, |
| | | 1H, O(C═O)NH), 3.13 (dt, J = 6.3/6.3 Hz, 2H, CH₂NH(C═O)NH), 3.00 (dt, J = 5.8/5.8 |
| | | Hz, 2H, CH₂NH(C═O)O), 1.38 (s, 9H, C(CH₃)₃). |
| 15k | 117-119 | δ 8.76 (s, 1H, NH(C═O)NHAr), 7.66 (s, 1H, aryl 2-H), 7.21 (dd, J = 7.9/7.9 Hz, 1H, |
| | | aryl 5-H), 7.17 (d, J = 8.2 Hz, 1H, aryl C—H), 6.92 (d, J = 7.7 Hz, 1H, aryl C—H), 6.87 |
| | | (t, J = 5.1 Hz, 1H, NH(C═O)NHAr), 6.23 (t, J = 5.3 Hz, 1H, O(C═O)NH), 3.11 (dt, J = |
| | | 6.1/6.1 Hz, 2H, CH₂NH(C═O)NH), 3.00 (dt, J = 5.8/5.8 Hz, 2H, CH₂NH(C═O)O), |
| | | 1.37 (s, 9H, C(CH₃)₃). |
| 15l | 165-167 | δ 8.70 (s, 1H, NH(C═O)NHAr), 7.41 (d, J = 8.9 Hz, 2H, aryl C—H), 7.25 (d, J = 8.9 |
| | | Hz, 2H, aryl C—H), 6.86 (t, J = 5.3 Hz, 1H, NH(C═O)NHAr), 6.20 (t, J = 5.5 Hz, 1H, |
| | | O(C═O)NH), 3.11 (dt, J = 6.4/6.4 Hz, 2H, CH₂NH(C═O)NH), 2.99 (dt, J = 5.8/5.8 Hz, |
| | | 2H, CH₂NH(C═O)O), 1.37 (s, 9H, C(CH₃)₃). |
| 15m | 136-138 | δ 8.06 (d, J = 8.2 Hz, 1H, aryl 6-H), 7.85 (s, 1H, NH(C═O)NHAr), 7.54 (dd, J = |
| | | 8.0/1.4 Hz, 1H, aryl 3-H), 7.27 (ddd, J = 7.8/7.8/1.4 Hz, 1H, aryl C—H), 7.14 (t, J = 5.4 |
| | | Hz, 1H, NH(C═O)NHAr), 6.85-6.90 (m, 2H, aryl C—H, O(C═O)NH), 3.12 (dt, J = |
| | | 6.4/6.4 Hz, 2H, CH₂NH(C═O)NH), 3.01 (dt, J = 5.8/5.8 Hz, 2H, CH₂NH(C═O)O), |
| | | 1.38 (s, 9H, C(CH₃)₃). |
| 15n | 122-124 | δ 8.76 (s, 1H, NH(C═O)NHAr), 7.81 (dd, J = 1.8/1.8 Hz, 1H, aryl 2-H), 7.21 (ddd, J = |
| | | 8.5/1.4/1.4 Hz, 1H, aryl C—H), 7.16 (dd, J = 7.8/7.8 Hz, 1H, aryl 5-H), 7.05 (ddd, J = |
| | | 7.7/1.2/1.2 Hz, 1H, aryl C—H), 6.86 (t, J = 5.3 Hz, 1H, NH(C═O)NHAr), 6.24 (t, J = |
| | | 5.5 Hz, 1H, O(C═O)NH), 3.11 (dt, J = 6.3/6.3 Hz, 2H, CH₂NH(C═O)NH), 3.00 (dt, J = |
| | | 5.8/5.8 Hz, 2H, CH₂NH(C═O)O), 1.37 (s, 9H, C(CH₃)₃). |
| 15o | 192-194 | δ 8.70 (s, 1H, NH(C═O)NHAr), 7.37 (s, 4H, aryl C—H), 6.86 (t, J = 5.3 Hz, 1H, |
| | | NH(C═O)NHAr), 6.20 (t, J = 5.5 Hz, 1H, O(C═O)NH), 3.11 (dt, J = 6.3/6.3 Hz, 2H, |
| | | CH₂NH(C═O)NH), 2.99 (dt, J = 5.8/5.8 Hz, 2H, CH₂NH(C═O)O), 1.37 (s, 9H, |
| | | C(CH₃)₃). |
| 15p | 149-151 | δ 7.96 (d, J = 8.2 Hz, 1H, aryl C—H), 7.80 (s, 1H, NH(C═O)NHAr), 7.60 (d, J = 7.9 |
| | | Hz, 1H, aryl C—H), 7.56 (dd, J = 8.1/8.1 Hz, 1H, aryl C—H), 7.17 (dd, J = 7.4/7.4 Hz, |
| | | 1H, aryl C—H), 7.03-7.10 (m, 1H, NH(C═O)NHAr), 6.82-6.90 (m, 1H, O(C═O)NH), |
| | | 3.08-3.18 (m, 2H, CH₂NH(C═O)NH), 2.95-3.06 (m, 2H, CH₂NH(C═O)O), 1.38 (s, |
| | | 9H, C(CH₃)₃). |
| 15q | 102-103 | δ 8.93 (s, 1H, NH(C═O)NHAr), 7.97 (s, 1H, aryl 2-H), 7.49 (d, J = 8.3 Hz, 1H, aryl 6- |
| | | H), 7.43 (dd, J = 7.6/7.6 Hz, 1H, aryl 5-H), 7.21 (d, J = 7.4 Hz, 1H, aryl 4-H), 6.87 (t, J = |
| | | 5.1 Hz, 1H, NH(C═O)NHAr), 6.28 (t, J = 5.2 Hz, 1H, O(C═O)NH), 3.13 (dt, J = |
| | | 6.3/6.3 Hz, 2H, CH₂NH(C═O)NH), 3.01 (dt, J = 5.8/5.8 Hz, 2H, CH₂NH(C═O)O), 1.37 |
| | | (s, 9H, C(CH₃)₃). |
| 15r | 194-196 | δ 8.99 (s, 1H, NH(C═O)NHAr), 7.59 (d, J = 9.1 Hz, 2H, aryl C—H), 7.55 (d, J = 9.1 Hz, |
| | | 2H, aryl C—H), 6.88 (t, J = 5.3 Hz, 1H, NH(C═O)NHAr), 6.31 (t, J = 5.5 Hz, 1H, |
| | | O(C═O)NH), 3.13 (dt, J = 6.3/6.3 Hz, 2H, CH₂NH(C═O)NH), 3.01 (dt, J = 5.8/5.8 Hz, |
| | | 2H, CH₂NH(C═O)O), 1.37 (s, 9H, C(CH₃)₃). |
| 21a | δ 8.81 (s, 1H, NH(C═O)NHAr), 7.62 (ddd, J = 13.7/7.5/2.6 Hz, 1H, aryl 2-H), 7.26 (ddd, J = | |
| | 10.6/9.5/9.2 Hz, 1H, aryl 5-H), 6.98-7.05 (m, 1H, aryl 6-H), 6.84 (t, J = 4.6 Hz, 1H, | |
| | NH(C═O)NHAr), 6.24 (t, J = 5.8 Hz, 1H, O(C═O)NH), 3.11 (dt, J = 6.2/5.8 Hz, 2H, | |
| | CH₂NH(C═O)NH), 2.99 (dt, J = 6.2/5.8 Hz, 2H, CH₂NH(C═O)O), 1.37 (s, 9H, C(CH₃)₃). | |
| 19 | 138-140 | (CDCl₃): δ 7.85 (br s, 1H, NH(C═S)NHPh), 7.42 (dd, J = 7.7/7.7 Hz, 2H, 3-H and 5-H |
| | | phenyl ring), 7.29 (dd, J = 7.3/7.3 Hz, 1H, 4-H phenyl ring), 7.22 (d, J = 7.7 Hz, 2H, 2- |
| | | H and 6-H phenyl ring), 6.85 (br s, 1H, O(C═O)NH), 4.90 (br s, 1H, NH(C═S)NHPh), |
| | | 3.74 (dt, J = 5.9/5.2 Hz, 2H, CH₂NH(C═S)NH), 3.32 (dt, J = 6.3/5.6 Hz, 2H, |
| | | CH₂NH(C═O)O), 1.34 (s, 9H, C(CH₃)₃). |

### General Procedure for Synthesis of Phenyl Substituted 1(2-aminoethyl)-3-(phenyl)urea hydrochlorides

The desired phenyl substituted Boc-protected phenylurea (compounds 15a-15r) was dissolved in MeOH (6 mL) with the aid of sonication and heat if necessary. This was then added to vigorously stirred concentrated aqueous HCl (5 mL) and stirred for 3 hours. All solvents were removed under vacuum and the resulting hydrochloride salts of the desired compounds were freeze-dried.

### 1-(2-aminoethyl)-3-(4-nitrophenyl)urea hydrochlorides (16s)

15s (1.66 g, 5.12 mmol) was dispersed in DCM/THF (1:1, 10 mL), with addition of MeOH to complete dissolution. To this stirred solution was added 4M HCl/dioxane (15 mL) and stirring continued at rt for 1 hour. Addition of excess petroleum ether 40-60 coused precipitation of the desired compound as a yellow amorphous solid, which was collected by filtration (vacuum) to give 1.123 g (84%) of 16s.

### Methyl 3-(3-(2-aminoethyl)ureido)benzoate hydrochloride (16t)

15t (2.343 g, 6.94 mmol) was dispersed in 3N HCl in MeOH (30 mL) and stirred at rt for 2 hours during which time the initially white suspension turned to a clear solution. LC-MS analysis indicated the reaction was complete. The mixture was concentrated to give 16t in quantitative yield as an off-white amorphous solid.

### Methyl 4-(3-(2-aminoethyl)ureido)benzoate hydrochloride (16u)

15u underwent deprotection as described in the synthesis of 16t to give 16u as a white amorphous solid in quantitative yield.

Table 5 lists the ¹H NMR spectral data for selected compounds from Scheme 2:

| Cpd/ | |
|---|---|
| m.p | ¹H NMR (DMSO-d₆) |
| 16a | δ 8.93 (s, 1H, NH(C═O)NHPh), 7.89 (br s, 3H, NH₃⁺), 7.41 (d, J = 7.6 Hz, 2H, 2-H and 6-H |
| 186-188 | phenyl ring), 7.22 (dd, J = 7.5/7.5 Hz, 2H, 3-H and 5-H phenyl ring), 6.90 (dd, J = 7.3/7.3 |
| | Hz, 1H, 4-H phenyl ring), 6.34 (t, J = 5.7 Hz, NH(C═O)NHPh), 3.30-3.34 (m, 2H, |
| | CH₂NH(C═O)NH), 2.83-2.93 (m, 2H, CH₂NH₃⁺). |
| 16b | δ 9.01 (s, 1H, NH(C═O)NHAr), 8.02 (br s, 3H, NH₃⁺), 7.18-7.25 (m, 2H, aryl 2-H, aryl C—H), |
| 185-187 | 7.09 (dd, J = 7.7/7.7 Hz, 1H, aryl 5-H), 6.67-6.75 (m, 2H, aryl C—H, NH(C═O)NHAr), |
| | 3.31 (dt, J = 6.2/6.2 Hz, 2H, CH₂NH(C═O)NH), 2.87 (t, J = 6.2 Hz, CH₂NH₃⁺), 2.23 (s, 3H, |
| | CH₃). |
| 16c | δ 8.95 (s, 1H, NH(C═O)NHAr), 8.01 (br s, 3H, NH₃⁺), 7.29 (d, J = 8.4 Hz, 2H, aryl 2-H and |
| 214-216 | 6-H), 7.02 (d, J = 8.4 Hz, 2H, aryl 3-H and 5-H), 6.63 (t, J = 5.8 Hz, 1H, NH(C═O)NHAr), |
| | 3.31 (dt, J = 6.2/6.2 Hz, 2H, CH₂NH(C═O)NH), 2.86 (t, J = 6.2 Hz, 2H, CH₂NH₃⁺), 2.20 (s, |
| | 3H, CH₃). |
| 16d | δ 8.05 (dd, J = 7.8/1.8 Hz, 1H, aryl 6-H), 8.03 (s, 1H, NH(C═O)NHAr), 8.00 (br s, 3H, |
| 156-158 | NH₃⁺), 7.19-7.26 (m, 1H, NH(C═O)NHAr), 6.96 (dd, J = 7.9/1.6 Hz, 1H, aryl 3-H), 6.88 |
| | (ddd, J = 7.4/7.4/1.8 Hz, 1H, aryl C—H), 6.84 (ddd, J = 7.8/7.8/1.7 Hz, 1H, aryl C—H), 3.82 (s, |
| | 3H, CH₃), 3.31 (dt, J = 6.1/6.1 Hz, 2H, CH₂NH(C═O)NH), 2.87 (tq, J = 5.8/5.8 Hz, |
| | CH₂NH₃⁺). |
| 16e | δ 9.03 (s, 1H, NH(C═O)NHAr), 7.95 (br s, 3H, NH₃⁺), 7.16 (dd, J = 2.2/2.2 Hz, 1H, aryl 2- |
| 183-188 | H), 7.11 (dd, J = 8.1/8.1 Hz, 1H, aryl 5-H), 6.89 (dd, J = 8.1/1.7 Hz, 1H, aryl 6-H), 6.61(t, J = |
| | 5.7 Hz, 1H, NH(C═O)NHAr), 6.48 (dd, J = 8.1/2.4 Hz, 1H, aryl 4-H), 3.69 (s, 3H, CH₃), |
| | 3.32 (dt, J = 6.2/6.2 Hz, 2H, CH₂NH(C═O)NH), 2.87 (tq, J = 5.9/5.9 Hz, CH₂NH₃⁺). |
| 16f | δ 8.75 (s, 1H, NH(C═O)NHAr), 7.93 (br s, 3H, NH₃⁺), 7.30 (d, J = 9.1 Hz, 2H, aryl 2-H and |
| 180-182 | 6-H), 6.81 (d, J = 9.1 Hz, 2H, aryl 3-H and 5-H), 6.47 (t, J = 5.8 Hz, 1H, NH(C═O)NHAr), |
| | 3.69 (s, 3H, CH₃), 3.33 (dt, J = 6.1/6.1 Hz, 2H, CH₂NH(C═O)NH), 2.86 (tq, J = 5.8/5.8 Hz, |
| | CH₂NH₃⁺). |
| 16g | δ 8.57 (d, J = 2.1 Hz, 1H, NH(C═O)NHAr), 8.05-8.10 (m, 4H, aryl C—H, NH₃⁺), 7.11-7.21 |
| 208-210 | (m, 2H, aryl 3-H, NH(C═O)NHAr), 7.08 (dd, J = 7.8/7.8 Hz, 1H, aryl C—H), 6.90-6.96 (m, |
| | 1H, aryl C—H), 3.34 (dt, J = 6.0/6.0 Hz, 2H, CH₂NH(C═O)NH), 2.88 (t, J = 6.2 Hz, 2H, |
| | CH₂NH₃⁺). |
| 16h | δ 9.42 (s, 1H, NH(C═O)NHAr), 8.00 (br s, 3H, NH₃⁺), 7.46 (ddd, J = 12.3/2.2/2.2 Hz, 1H, |
| 184-186 | aryl 2-H), 7.23 (ddd, J = 8.2/8.2/7.0 Hz, 1H, aryl 5-H), 7.06 (dd, J = 8.2/1.8 Hz, 1H, aryl 6- |
| | H), 6.76 (t, J = 5.8 Hz, 1H, NH(C═O)NHAr), 6.69 (ddd, J = 8.7/8.7/2.5 Hz, 1H, aryl 4-H), |
| | 3.33 (dt, J = 6.2/6.2 Hz, 2H, CH₂NH(C═O)NH), 2.87 (tq, J = 5.4/5.4 Hz, 2H, CH₂NH₃⁺). |
| 16i | δ 9.17 (s, 1H, NH(C═O)NHAr), 8.02 (br s, 3H, NH₃⁺), 7.42 (dd, J = 9.2/5.0 Hz, 2H, aryl 2-H |
| 203-205 | and 6-H), 7.05 (dd, J = 8.9/8.9 Hz, 2H, aryl 3-H and 5-H), 6.62-6.73 (m, 1H, |
| | NH(C═O)NHAr), 3.27-3.38 (m, 2H, CH₂NH(C═O)NH), 2.86 (tq, J = 5.7/5.7 Hz, 2H, |
| | CH₂NH₃⁺). |
| 16j | δ 8.44 (br s, 1H, NH(C═O)NHAr), 8.12 (dd, J = 8.3/1.5 Hz, 1H, aryl C—H), 8.07 (br s, 3H, |
| 205-207 | NH₃⁺), 7.52 (t, J = 5.6 Hz, 1H, NH(C═O)NHAr), 7.40 (dd, J = 8.0/1.5 Hz, 1H, aryl C—H), 7.24 |
| | (dd, J = 7.8/1.4 Hz, 1H, aryl C—H), 6.96 (dd, J = 7.8/1.5 Hz, 1H, aryl C—H), 3.31-3.39 (m, |
| | 2H, CH₂NH(C═O)NH), 2.89 (br s, 2H, CH₂NH₃⁺). |
| 16k | δ 9.25 (s, 1H, NH(C═O)NHAr), 7.86 (br s, 3H, NH₃⁺), 7.66-7.70 (m, 1H, aryl 2-H), 7.20- |
| 193-195 | 7.27 (m, 2H, aryl C—H), 6.94 (ddd, J = 6.7/2.2/2.2 Hz, 1H, aryl C—H), 6.62 (t, J = 5.8 Hz, 1H, |
| | NH(C═O)NHAr), 3.32 (dt, J = 6.2/6.2 Hz, 2H, CH₂NH(C═O)NH), 2.88 (tq, J = 5.8/5.8 Hz, |
| | 2H, CH₂NH₃⁺). |
| 16l | δ 9.25 (s, 1H, NH(C═O)NHAr), 7.95 (br s, 3H, NH₃⁺), 7.45, 7.27 (d, J = 8.9 Hz, 2 x 2H, aryl |
| 223-225 | C—H), 6.67 (t, J = 5.8 Hz, 1H, NH(C═O)NHAr), 3.32 (dt, J = 6.2/6.2 Hz, 2H, |
| | CH₂NH(C═O)NH), 2.87 (tq, J = 5.7/5.7 Hz, 2H, CH₂NH₃⁺). |
| 16m | 8.09 (br s, 3H, NH₃⁺), 7.99-8.06 (m, 2H, NH(C═O)NHAr, aryl C—H), 7.54-7.60 (m, 2H, |
| 195-197 | NH(C═O)NHAr, aryl C—H), 7.28 (dd, J = 7.8/1.4 Hz, 1H, aryl C—H), 6.91 (dd, J = 8.0/1.6 Hz, |
| | 1H, aryl C—H), 3.29-3.38 (m, 2H, CH₂NH(C═O)NH), 2.89 (tq, J = 5.6/5.6 Hz, 2H, |
| | CH₂NH₃⁺). |
| 16n | δ 9.41 (s, 1H, NH(C═O)NHAr), 7.98 (br s, 3H, NH₃⁺), 7.82 (dd, J = 1.9/1.9 Hz, 1H, aryl 2- |
| 196-198 | H), 7.28 (ddd, J = 8.2/1.9/0.9 Hz, 1H, aryl C—H), 7.17 (dd, J = 8.0/8.0 Hz, 1H, aryl 5-H), |
| | 7.06 (ddd, J = 7.9/1.9/1.0 Hz, 1H, aryl C—H), 6.76 (t, J = 5.8 Hz, 1H, NH(C═O)NHAr), 3.32 |
| | (dt, J = 6.3/6.3 Hz, 2H, CH₂NH(C═O)NH), 2.87 (t, J = 5.8 Hz, 2H, CH₂NH₃⁺). |
| 16o | δ 9.30 (s, 1H, NH(C═O)NHAr), 7.98 (br s, 3H, NH₃⁺), 7.37-7.43 (m, 4H, aryl C—H), 6.71 (t, |
| 229-231 | J = 5.8 Hz, 1H, NH(C═O)NHAr), 3.32 (dt, J = 6.3/6.3 Hz, 2H, CH₂NH(C═O)NH), 2.87 (t, J = |
| | 5.6 Hz, 2H, CH₂NH₃⁺). |
| 16p | 8.09 (br s, 3H, NH₃⁺), 8.01 (s, 1H, NH(C═O)NHAr), 7.94 (d, J = 8.3 Hz, 1H, aryl 6-H), 7.62 |
| 153-155 | (d, J = 7.9 Hz, 1H, aryl 3-H), 7.58 (dd, J = 8.1/8.1 Hz, 1H, aryl C—H), 7.46 (t, J = 5.5 Hz, 1H, |
| | NH(C═O)NHAr), 7.21 (dd, J = 7.6/7.6 Hz, 1H, aryl C—H), 3.32-3.39 (m, 2H, |
| | CH₂NH(C═O)NH), 2.89 (t, J = 6.3 Hz, 2H, CH₂NH₃⁺). |
| 16q | 9.59 (s, 1H, NH(C═O)NHAr), 7.92-8.04 (m, 4H, aryl 2-H, NH₃⁺), 7.55 (d, J = 8.5 Hz, 1H, |
| 175-177 | aryl 6-H), 7.45 (dd, J = 7.8/7.8 Hz, 1H, aryl 5-H), 7.22 (d, J = 7.6 Hz, 1H, aryl 4-H), 6.80 (t, |
| | J = 5.7 Hz, 1H, NH(C═O)NHAr), 3.30-3.40 (m, 2H, CH₂NH(C═O)NH), 2.89 (t, J = 6.1 Hz, |
| | 2H, CH₂NH₃⁺). |
| 16r | δ 9.66 (s, 1H, NH(C═O)NHAr), 8.02 (br s, 3H, NH₃⁺), 7.62 (d, J = 8.7 Hz, 2H, aryl 2-H and |
| 203-206 | 6-H), 7.57 (d, J = 8.9 Hz, 2H, aryl 3-H and 5-H), 6.87 (t, J = 5.8 Hz, 1H, NH(C═O)NHAr), |
| | 3.35 (dt, J = 6.1/6.1 Hz, 2H, CH₂NH(C═O)NH), 2.89 (t, J = 6.2 Hz, 2H, CH₂NH₃⁺). |

### tert-Butyl 2-(2-phenylacetamido)ethylcarbamate (17)

14 (1 g, 6.25 mmol) and TEA (958 μL, 6.88 mmol, 1.1 eq) were dissolved in dry DCM (20 mL) and cooled to 0° C. under a nitrogen atmosphere. Phenyl acetyl chloride (826 μL, 6.25 mmol, 1 eq) was added and the mixture stirred at rt for 2 hours. The TEA.HCl salt was filtered before concentration of the filtrate under reduced pressure. The crude residue was dissolved in EtOAc (50 mL) and washed with acidified water (25 mL, pH 4 adjusted using aqueous 1M KHSO₄), aqueous 2M NaOH (25 mL) and water (25 mL). The organic layer was concentrated under reduced pressure to give 1.460 g of white solid requiring no further purification.

### General Procedure for Synthesis of tert-butyl 2-(2-(hydroxyphenyl)acetamido)ethylcarbamates (17a-c)

2-, 3- or 4-hydroxyphenylacetic acid (2.59 g, 17.02 mmol) was dissolved in DCM (40 mL) with the aid of sonication and heat where necessary. To this was added DCC (3.86 g, 18.73 mmol, 1.1 eq) and the mixture was stirred for 30 min. 14 (3.00 g, 18.73 mmol, 1.1 eq) was then added and the mixture was stirred for 48 h. The reaction mixture was filtered under vacuum and the filtrate washed with DCM (3×20 mL). The combined organic filtrates were washed with acidified water (acidified using aqueous 1 M KHSO₄, 2×20 mL) before concentration under reduced pressure. After solvent removal, 17a-c were purified using column chromatography (eluent EtOAc/Pet Ether).

Table 6 lists the ¹H NMR spectral data for selected compounds from Scheme 2:

| | m.p/ | |
|---|---|---|
| Cpd | ° C. | ¹H NMR |
| 17 | 133-135 | δ 7.25 -7.39 (m, 5H, phenyl CH), 6.05 (br s, 1H, amide NH), 4.87 (br s, 1H, |
| | | carbamate NH), 3.55 (s, 2H, CH₂C═O), 3.29-3.33 (m, 2H, CH₂NH), 3.18-3.22 |
| | | (m, 2H, CH₂NH), 1.42 (s, 9H, (CH₃)₃). |
| 17a | 125-127 | δ 9.85 (br s, 1H, phenol OH), 7.16 (ddt, J = 7.9/7.9/1.7 Hz, 2H, aryl 4H), 6.12 (br |
| | | s, 1H, NH), 7.10 (dd, J = 7.2/1.2 Hz, 1H, aryl 6H) 6.96 (dd, J = 8.0/0.8 Hz, 2H, |
| | | aryl 3H), 6.81 (ddt, J = 7.4/7.4/1.2 Hz, 1H, aryl 5H), 4.95-5.05 (m, 1H, NH), |
| | | 3.55 (s, 2H, CH₂Ar), 3.20-3.39 (m, 4H, NH₂CH₂CH₂NH₂), 1.44 (s, 9H, (CH₃)₃). |
| 17b | | (DMSO-d₆): δ 9.26 (br s, 1H, phenol OH), 7.99 (br s, 1H, NH), 7.05 (dd, J = |
| | | 7.6/7.6 Hz, 1H, aryl 5H), 6.75-6.85 (m, 1H, NH), 6.52-6.71 (m, 3H, aryl 2H, |
| | | 4H, 6H), 3.28 (s, 2H, CH₂Ar), 2.93-3.11 (m, 4H, NHCH₂CH₂NH), 1.37 (s, 9H, |
| | | (CH₃)₃). |
| 17c | 121-123 | (DMSO-d₆): δ 9.18 (br s, 1H, phenol OH), 7.85-7.95 (m, 1H, NH), 7.02 (d, J = |
| | | 8.5 Hz, 2H, aryl 2H, 6H), 6.73-6.83 (m, 1H, NH), 6.66 (d, J = 8.5 Hz, 2H, aryl |
| | | 3H, 5H), 3.24 (s, 2H, CH₂Ar), 2.91-3.09 (m, 4H, NHCH₂CH₂NH₂), 1.37 (s, 9H, |
| | | (CH₃)₃). |

### 2-(2-Phenylacetamido)ethylammonium trifluoroacetate (18)

17 (1.310 g, 4.71 mmol) was dissolved in TFA/DCM (20 mL 1:1) and stirred for 2 hours at rt. Removal of volatiles under reduced pressure gave 1.518 g of semi-solid requiring no further purification.

### General Procedure for Synthesis of Phenyl Substituted N-(2-aminoethyl)-2-(hydroxyphenyl)acetamide hydrochlorides and N-(4-amino) hydroxyphenylbutanamine hydrochlorides (18a-c, 26 a-c)

17a-c or 25a-b were dissolved in the minimum required volume of MeOH, with the aid of sonication and heat where necessary. The methanolic solution was then added to an equivalent volume of stirred 4M HCl in dioxane. The reaction mixture was stirred for 3 h, before removal of solvent under reduced pressure. The resulting hydrochloride salts of the amines were freeze dried and required no further purification.

Table 7 lists the ¹H NMR spectral data for selected compounds from Scheme 2:

| Cpd | ¹H NMR (DMSO-d₆) |
|---|---|
| 18 | δ 8.30 (t, J = 5.4 Hz, 1H, amide NH), 7.90 (br s, 3H, NH₃⁺), 7.20-7.31 (m, 5H, phenyl |
| | CH), 3.43 (s, 2H, CH₂C═O), 3.28 (dt, J = 6.0/6.4 Hz, 2H, CH₂NH), 2.84-2.90 (m, 2H, |
| | CH₂NH₃⁺). |
| 18a | δ 8.45 (br s, 1H, phenol OH), 8.27 (t, J = 5.5 Hz, 1H, NH(C═O)), 8.08 (br s, 3H, NH₃⁺), |
| | 7.08 (dd, J = 7.3/1.4 Hz 1H, aryl 6H), 7.04 (ddd, J = 7.7/7.7/1.7 Hz, 1H, aryl 4H), 6.81 (dd, |
| | J = 8.0/1.1 Hz, 1H, aryl 3H), 6.71 (ddd, J = 7.4/7.4/1.1 Hz, 1H, aryl 5H), 3.40 (s, 2H, |
| | NH(C═O)CH₂), 3.30 (dt, J = 6.4/5.9 Hz, 2H, CH₂NH(C═O)), 2.84 (dt, J = 6.2/6.0 Hz, 2H, |
| | NH₃⁺CH₂). |
| 18b | δ 8.42 (t, J = 5.4 Hz, 1H, NH(C═O)), 8.13 (s, 3H, NH₃⁺), 7.05 (dd, J = 7.7/7.7 Hz, 1H, aryl |
| | 5-H), 6.52-6.78 (m, 3H, aryl 2-H, 4-H, 6-H), 5.75 (s, 1H, OH), 3.36 (s, 1H, |
| | NH(C═O)CH₂), 3.28 (dt, J = 6.5/6.1 Hz, 2H, CH₂NH(C═O)), 2.84 (dt, J = 6.0/6.0 Hz, 2H, |
| | NH₃⁺CH₂). |
| 18c | δ 8.33 (t, J = 5.4 Hz, 1H, NH(C═O)), 8.09 (s, 3H, NH₃⁺), 7.04 (d, J = 8.5 Hz, 2H, aryl 2-H, |
| | 6-H), 6.68 (d, J = 8.4 Hz, 2H, aryl 3-H, 5-H), 5.75 (s, 1H, OH), 3.23-3.34 (m, 4H, |
| | CH₂NH(C═O), NH(C═O)CH₂), 2.83 (dt, J = 6.1/5.9 Hz, 2H, NH₃⁺CH₂). |

### tert-Butyl 2-(3-phenylthioureido)ethylcarbamate (19)—see Table 4

### 1-(2-Aminoethyl)-3-phenylthiourea hydrochloride (20)

19 (882 mg, 2.99 mmol) was stirred overnight in 4 M HCl in dioxane (20 mL). Removal of all volatiles under reduced pressure gave 592 mg of cream crystalline solid requiring no further purification, listed in Table 7 below:

### tert-Butyl 2-(benzylsulfonamido)ethylcarbamate (21)

14 (500 mg, 3.12 mmol) and TEA (478 μL, 3.43 mmol, 1.1 eq) were dissolved in dry DCM (10 mL) under a nitrogen atmosphere. Phenylmethanesulfonyl chloride (595 mg, 3.12 mmol, 1 eq) in DCM (5 mL) was added dropwise whilst cooling the mixture over an ice bath. After stirring at rt overnight, the crude mixture was diluted to 30 mL with DCM before washing with aqueous 1 M KHSO₄ (20 mL), aqueous 1 M NaOH (20 mL) and water (20 mL). Removal of all volatiles under reduced pressure gave 761 mg of white solid requiring no further purification, listed in Table 8 below:

| Cpd | m.p | ¹H NMR |
|---|---|---|
| 20 | 110-114 | (CDCl₃): δ 7.34-7.42 (m, 5H, phenyl C—H), 4.78-4.93 (m, 2H, 2 x N—H), 4.25 (s, |
| | | 2H, SO₂CH₂), 3.11-3.23 (m, 2H, C═ONHCH₂), 2.96-3.08 (m, 2H, CH₂NHSO₂), |
| | | 1.43 (s, 9H, C(CH₃)₃) |
| 21 | 183-185 | (DMSO-d₆): δ 10.10 (s, 1H, NH(C═S)NHPh), 8.22 (t, J = 5.5 Hz, NH(C═S)NHPh), |
| | | 8.08 (br s, 3H, NH₃⁺), 7.46 (dd, J = 8.6/1.1 Hz, 2H, 2-H and 6-H phenyl ring), 7.32 |
| | | (dd, J = 7.4/7.4 Hz, 2H, 3-H and 5-H phenyl ring), 7.11 (dd, J = 7.4/7.4 Hz, 1H, 4-H |
| | | phenyl ring), 3.74 (dt, J = 6.4/5.9 Hz, 2H, CH₂NH(C═O)NH), 2.95-3.06 (m, 2H, |
| | | CH₂NH₃⁺). |

### 21a—see Table 4

### Phenyl-2-(tert-butyloxycarbonyl)aminoethylcarbamate (21b)

14 (1.000 g, 6.25 mmol) and TEA (759 mg, 1.045 mL, 7.5 mmol, 1.2 eq) were dissolved in dry DCM (20 mL) under a nitrogen atmosphere. Phenylchloroformate (1.076 g, 862 μL, 6.87 mmol, 1.1 eq) was added and the mixture stirred for 1 hour. After confirmation of total amine consumption by TLC, the mixture was diluted to 50 mL with DCM before washing with aqueous 1 M NaOH (1×50 mL), and water (1×50 mL). Removal of all volatiles gave 1.521 g of cream solid which was used without any further purification, listed in Table 9 below:

### General Procedure for the Synthesis of Phenyl Substituted tert-butyl 2-((phenylcarbonyl)amino)ethylcarbamates (21c-h)

14 (1 eq) and the appropriate substituted benzoic acid (1 eq) were dissolved in DCM, before cooling to 0° C. EDC (1.5 eq) in DCM (2 mL) was added and the mixture stirred vigorously overnight at rt. The reaction mixture was diluted to 25 mL with DCM before washing with acidified water (20 mL, acidified with aqueous KHSO₄ solution to pH4), and distilled water (20 mL). Concentration of the organic layer and subsequent purification via FCC (eluent EtOAc/PE, various compositions) gave the desired compounds listed in Table 9:

### tert-Butyl 2-(3-(3-chloro-4-methoxyphenyl)ureido)ethylcarbamate (21i)

Refer to the general procedure for synthesis of phenyl substituted tert-butyl 2-(3-phenylureido)ethylcarbamates.

Table 9 lists the ¹H NMR spectral data for selected compounds from Figure 2:

| Cpd | m.p | ¹H NMR (DMSO-d₆): |
|---|---|---|
| 21b | IR: 3333 (carbamate N—H, str), 2977 (alkyl, C—H, str), 1716, 1689 (carbamate C═O, str), 1526 | |
| | (aryl, str), 1366 (C(CH₃)₃, str), 769, 699 (aryl C—H bend, phenyl ring). | |
| 21c | δ 12.45 (br s, 1H, OH), 7.90 (br s, 1H, NH(C═O)), 7.48 (d, J = 7.2 Hz, 1H, aryl 6H), 7.30 | |
| | (ddd, J = 7.8/7.8/1.1 Hz, 1H, aryl 4H), 6.90 (d, J = 8.1 Hz, 1H, aryl 3H), 6.75 (dd, J = 7.3/7.3 | |
| | Hz, 1H, aryl 5H), 5.38 (br, s, 1H, (C═O)NH), 3.44-3.51 (m, 2H, NHCH₂), 3.30-3.38 (m, 2H, | |
| | NHCH₂), 1.38 (s, 9H, C(CH₃)₃). | |
| 21d | δ 8.60 (br, s, 1H, OH), 7.37-7.53 (m, 2H, NH(C═O), aryl 6H), 7.13-7.25 (m, 2H, aryl 2H, | |
| | 5H), 6.96 (d, J = 7.6 Hz, 1H, aryl 4H), 5.33-5.46 (m, 1H, (C═O)NH), 3.48 (dt, J = 5.3/5.9 | |
| | Hz, 2H, CH₂NH), 3.25-3.39 (m, 2H, CH₂NH), 1.38 (s, 9H, C(CH₃)₃). | |
| 21e | δ 7.65 (d, J = 7.8 Hz, 2H, aryl 3H, 5H), 7.13 (br s, 1H, NH(C═O)), 6.83 (d, J = 8.8 Hz, 2H, aryl | |
| | 2H, 6H), 5.12 (br s, 1H, (C═O)NH), 3.53 (dt, J = 5.3/5.3 Hz, 2H, CH₂NH), 3.34-3.44 (m, 2H, | |
| | NHCH₂), 1.43 (s, 9H, C(CH₃)₃). | |
| 21f | 116-117 | δ 8.04 (t, J = 7.9 Hz, 1H, NH(C═O)), 7.42-7.50 (m, 1H, aryl 6H), 7.22-7.29 (m, |
| | | 1H, aryl 4H), 7.07-7.18 (m, 2H, aryl 3H, 5H), 4.90 (br, s, 1H, (C═O)NH), 3.60 |
| | | (dt, J = 5.5/5.5 Hz, 2H, CH₂NH), 3.39 (dt, J = 5.5/5.5 Hz, 2H, NHCH₂), 1.40 (s, |
| | | 9H, C(CH₃)₃). |
| 21g | 109.5-110.5 | δ 7.51-7.63 (m, 2H, aryl 2H, 6H), 7.28-7.44 (m, aryl 5H, NH(C═O)), 7.14- |
| | | 7.21 (m, 1H, aryl 4H), 5.00 (br s, 1H, (C═O)NH), 3.51-3.59 (m, 2H, CH₂NH), |
| | | 3.37-3.46 (m, 2H, NHCH₂), 1.43 (s, 9H, C(CH₃)₃) |
| 21h | 116-117 | δ 7.83 (dd, J = 8.3/5.1 Hz, 2H, aryl 2H, 6H), 7.25 (br s, 1H, NH(C═O)), 7.08 |
| | | (dd, J = 8.6/8.6 Hz, 2H, aryl 3H, 5H), 5.00 (br s, 1H, (C═O)NH), 3.51-3.59 (m, |
| | | 2H, CH₂NH), 3.37-3.45 (m, 2H, NHCH₂), 1.42 (s, 9H, C(CH₃)₃). |

### N-(Aminoethyl)benzylsulfonamide hydrochloride (22)

Deprotection of 21 (704 mg, 2.24 mmol) was achieved as described for 19, giving 552 mg of cream solid requiring no further purification.

### 1-(2-Aminoethyl)-3-(3,4-difluorophenyl)urea hydrochloride (22a)

21a (1.966 g, 6.23 mmol) was dissolved in MeOH (10 mL) with vigorous stirring. To this was added 4 M HCl/dioxane (40 mL) and the solution stirred for 4 hours. After removal of all solvents in vacuo, the crude residue was triturated with toluene and dried to give a beige solid in quantitative yield.

### Phenyl 2-aminoethylcarbamate hydrochloride (22b)

21b was dissolved in Et₂O (15 mL) and 4 M HCl/dioxane (15 mL). After 10 minutes the formed precipitate was filtered (suction) and washed with Et₂O to give a white solid in quantitative yield.

Table 10 lists the ¹H NMR spectral data for selected compounds from Figure 2:

| Cpd | m.p | ¹H NMR (DMSO-d₆): |
|---|---|---|
| 22 | 191.5-193.5 | (CDCl₃): δ 8.08 (br s, 3H, NH₃⁺), 7.45 (t, J = 5.9 Hz, 1H, NHSO₂), 7.33-7.42 (m, |
| | | 5H, phenyl C—H), 4.40 (s, 2H, SO₂CH₂), 3.16 (dt, J = 6.8/5.9 Hz, 2H, CH₂NHSO₂), |
| | | 2.77-2.90 (m, 2H, NH₃⁺CH₂). |
| 22a | 191-193 | (DMSO-d₆): δ 9.51 (s, 1H, NH(C═O)NHAr), 8.05 (br s, 3H, NH₃⁺), 7.64 (ddd, J = |
| | | 14.0/7.8/2.6 Hz, 1H, aryl 2-H), 7.27 (ddd, J = 10.4/9.2/9.2 Hz, 1H, aryl 5-H), 7.02- |
| | | 7.10 (m, 1H, aryl 6-H), 6.78 (br s, 1H, NH(C═O)NHAr), 3.33 (t, J = 6.2/Hz, 2H, |
| | | CH₂NH(C═O)NH), 2.86 (tq, J = 5.8/5.8 Hz, 2H, CH₂NH₃⁺). |
| 22b | 166-168 | (DMSO-d₆): δ 8.23 (br s, 3H, NH₃⁺), 7.97 (t, J = 5.3 Hz, 1H, carbamate NH), 7.38 |
| | | (dd, J = 7.7/7.7 Hz, 2H, aryl 3-H and 5-H), 7.20 (dd, J = 7.7/7.1 Hz, 1H, aryl 4-H), |
| | | 7.13 (d, J = 7.7 Hz, 2H, aryl 2-H and 6-H), 3.35 (dt, J = 6.1/6.1 Hz, 2H, CH₂NH), |
| | | 2.86-3.01 (m, 2H, CH₂NH₃⁺). |

### General Procedure for Synthesis of Phenyl Substituted N-(2-aminoethyl)benzamide hydrochlorides (22c-h)

Each phenyl substituted tert-butyl 2-((phenylcarbonyl)amino)ethylcarbamate (0.7-1.5 mmol) (21c-h) was dissolved in distilled water (3 mL) before adding conc. HCl (2 mL) with care. The reaction mixture was then stirred for 2 hours. Where precipitates formed (fluorine containing analogues) these were collected by filtration (vacuum). In other cases, products were isolated by drying overnight in a freeze-drier.

### 1-(2-aminoethyl)-3-(3-chloro-4-methoxyphenyl)urea hydrobromide and 1-(2-aminoethyl)-3-(3-chloro-4-hydroxyphenyl)urea hydrobromide (mixture of compounds) (22i)

21i (500 mg, 1.45 mmol) was dissolved in dry DCM (8 mL) under an atmosphere of nitrogen, and cooled over an ice bath. 1M BBr₃ in DCM (7.6 mL, 5.2 eq) was added with care, before allowing the mixture to warm to rt and stir for 40 minutes. LCMS analysis indicated two product peaks had formed, so the mixture was stirred for a further 20 minutes before quenching with MeOH (over an ice bath) and concentrating under reduced pressure. The crude product was passed through a silica plug with DCM, followed by 1M NH₃ in MeOH/DCM (1:3). The product was found to be a mixture of both the methoxy and demthylated compounds, with no Boc group present. The mixed product was redissolved in dry DCM (25 mL), and 1M BBr₃ in DCM added (3.5 mL), with overnight stirring at rt. Final after quenching and concentrating as before the crude mixture was used without further purification,

Table 11 lists the ¹H NMR spectral data for selected compounds from Figure 2:

| Cpd | m.p | ¹H NMR (DMSO-d₆) |
|---|---|---|
| 22c | δ 12.40 (br s, 1H, OH), 9.09 (t, J = 5.9 Hz 1H, NH(C═O)), 8.10 (br s, 3H, NH₃⁺), 7.95 (dd, J = | |
| | 8.0/1.4 Hz, 1H, aryl 6H), 7.41(ddd, J = 7.7/7.7/1.6 Hz, 1H, aryl 4H), 6.86-6.95 (m, 2H, aryl | |
| | 3H, 5H), 3.57 (dt, J = 6.1/6.6 Hz, 2H, CH₂NH), 3.01 (dt, J = 5.9/5.9 Hz, 2H, NH₃⁺CH₂). | |
| 22d | δ 8.60 (t, J = 5.7 Hz, 1H, NH(C═O)), 8.05 (br s, 3H, NH₃⁺), 7.33 (d, J = 7.7 Hz, 1H, aryl 6H), | |
| | 7.28 (dd, J = 2.0/2.0 Hz, 1H, aryl 2H), 7.24 (dd, J = 8.2/8.2 Hz, 1H, aryl 5H), 6.93 (m, 1H, aryl | |
| | 4H), 3.49 (dt, J = 6.1/5.7 Hz, 2H, CH₂NH), 2.96 (tq, J = 6.1/5.7 Hz, 2H, NH₃⁺CH₂). | |
| 22e | 222-223 | δ 10.06 (br s, 1H, OH), 8.49 (t, J = 5.5 Hz, 1H, NH(C═O)), 8.00 (br s, 3H, NH₃⁺), |
| | | 7.78 (d, J = 8.6 Hz, 2H, aryl 2H, 6H), 6.82 (d, J = 8.6 Hz, 2H, aryl 3H, 5H), 3.48 (dt, J = |
| | | 6.1/5.8 Hz, 2H, CH₂NH), 2.90-3.03 (m, 2H, NH₃⁺CH₂). |
| 22f | 167-168 | δ 8.52 (br s, 1H, NH(C═O)), 8.16 (br s, 3H, NH₃⁺), 7.74 (dt, J = 7.2/7.2/1.8 Hz, 1H, |
| | | aryl 6H), 7.51-7.59 (m, 1H, aryl 4H), 7.26-7.33 (m, 2H, aryl 3H, 5H), 3.53 (dt, J = |
| | | 6.2/5.9 Hz, 2H, CH₂NH), 2.90-3.01 (m, 2H, NH₃⁺CH₂). |
| 22g | 189-190 | δ 8.88-8.99 (m, 1H, NH(C═O)), 8.15 (br s, 3H, NH₃⁺), 7.72-7.83 (m, 2H, aryl 2H, |
| | | 6H), 7.58-7.57 (m, 1H, aryl 4H or 5H), 7.35-7.42 (m, 1H, aryl 4H or 5H), 3.53 (dt, |
| | | J = 6.1/5.6 Hz, 2H, CH₂NH), 2.94-3.05 (m, 2H, NH₃⁺CH₂). |
| 22h | 213-214 | δ 8.85 (t, J = 5.4 Hz, 1H, NH(C═O)), 8.15 (br s, 3H, NH₃⁺), 8.01 (dd, J = 8.8/5.6 Hz, |
| | | 2H, aryl 2H, 6H), 7.30 (dd, J = 8.8/8.8 Hz, 2H, aryl 3H, 5H), 3.53 (dt, J = 6.1/5.9 Hz, |
| | | 2H, CH₂NH), 2.93-3.04 (m, 2H, NH₃⁺CH₂). |

### tert-Butyl 3-carboxypropylcarbamate (24)

23 (3.946 g, 38.27 mmol) and NaHCO₃ (7.07 g, 84.19 mmol, 2.2 eq) were dissolved in water/THF (4:1, 100 mL). Boc₂O (9.186 g, 42.09 mmol, 1.1 eq) was added and the mixture stirred at rt for 48 hours. THF was removed under reduced pressure before washing the remaining aqueous mixture with DCM (2×50 mL). The aqueous layer was then acidified using 2M aqueous HCl to pH 4 before extraction with DCM (4×30 mL). The organic layers were combined and concentrated to give 6.711 g of clear colourless oil requiring no further purification.

### tert-Butyl 3-(phenylcarbamoyl)propylcarbamate (25)

24 (921 mg, 4.53 mmol) and DCC (1.028 g, 4.98 mmol, 1.1 eq) were dissolved in DCM (25 mL) and stirred for 30 minutes. Aniline (454 μL, 4.98 mmol, 1.1 eq) was added and the mixture stirred at rt for 48 hours. The reaction mixture was diluted to 50 ml with DCM before washing with acidified water (30 ml, pH 4 adjusted using aqueous 1M KHSO₄), saturated aqueous NaHCO₃ (30 mL) and brine (30 mL). The organic layer was concentrated under reduced pressure and the crude residue purified via column chromatography (eluent EtOAc/Hexanes 10:90 to 80:20 over 10 column volumes). 25 was recrystallised from MeCN as 700 mg of crystalline white solid.

### General Procedure for Synthesis of tert-butyl 3-(hydroxyphenylcarbamoyl)propylcarbamates (25a-b)

24 (3.00 g, 14.76 mmol) was dissolved in DCM (40 mL). DCC (3.35 g, 16.24 mmol, 1.1 eq) was added, and the mixture was stirred for 30 mins. 2- or 3-aminophenol (1.77 g, 16.24 mmol, 1.1 eq) in DCM (10 mL) was then added, and the reaction mixture stirred for 48 h. After isolation of the precipitate by filtration (suction), and washing with DCM (3×20 mL), the combined filtrates were washed with extracted with acidified water (acidified using aqueous 1 M KHSO₄, 2×20 mL) before concentration of the organic layer under reduced pressure. After solvent removal, the products were purified using column chromatography (eluent EtOAc/Pet Ether).

### tert-Butyl 3-(phenoxycarbonyl)propylcarbamate (25c)

24 (2.000 g, 9.84 mmol) and DCC (2.232 g, 10.82 mmol, 1.1 eq) were dissolved in DCM (30 mL) and stirred for 30 minutes. Phenol (1.018 g, 10.82 mmol, 1.1 eq) was added and the mixture stirred for 48 hours. TLC monitoring indicated slow progression of the reaction, thus DMAP (122 mg, 1 mmol, 0.1 eq) was added and the reaction left to stir for a further 24 hours. The precipitated N,N′-dicyclohexylurea was filtered (suction) and the filtrate diluted to 50 mL with DCM before washing with acidified water (1×30 mL, water acidified to pH 4 with aqueous 1 M KHSO₄ solution) and aqueous 0.5 M NaOH (1×30 mL). After concentrating the organic layer, FCC (eluent EtOAc/hexanes 20:80) was required to afford 1.894 g of white crystalline solid.

Table 12 lists the ¹H NMR spectral data for selected compounds from Figure 2:

| Cpd | m.p | ¹H NMR |
|---|---|---|
| 24 | (CDCl₃): δ 10.13 (br s, 1H, CO₂H), 5.05-5.15 (br m, 1H, NH), 2.90-3.13 (br m, 2H, | |
| | NHCH₂), 2.24 (t, J = 7.3 Hz, 2H, CH₂CO₂H), 1.68 (tt, J = 7.0/7.0 Hz, 2H, CH₂CH₂CH₂), 1.23- | |
| | 1.38 (m, 9H, (CH₃)₃) | |
| 25 | 141-144 | (CDCl₃): δ 8.71 (br s, 1H, amide NH), 7.60 (d, J = 7.8 Hz, 2H, 2-H and 6-H phenyl |
| | | ring), 7.31 (dd, J = 7.6/7.6 Hz, 2H, 3-H and 5-H phenyl ring), 7.08 (dd, J = 7.4/7.4 Hz, |
| | | 1H, 4-H phenyl ring), 4.81 (br s, 1H, carbamate NH), 3.25 (dt, J = 6.0/6.0 Hz, 2H, |
| | | NHCH₂), 2.38 (t, J = 6.5 Hz, 2H, CH₂CONH), 1.88 (tt, J = 6.4 Hz, 2H (CH₂CH₂CH₂), |
| | | 1.46 (s, 9H, (CH₃)₃). |
| 25a | (DMSO-d₆): δ 9.71 (br s, 1H, phenol OH), 9.22 (br s, 1H, amide NH), 7.69 (d, J = 8.5 Hz, 1H, | |
| | aryl 6H), 6.89-6.97 (m, 1H, aryl CH), 6.79-6.88 (m, 2H, aryl 3H, carbamate NH), 6.75 | |
| | (ddd, J = 7.8/7.8/1.2 Hz, 1H, aryl CH), 2.96 (dt, J = 6.5/6.5 Hz, 2H, NHCH₂), 2.37 (t, J = 7.2 | |
| | Hz, 2H, CH₂(C═O)), 1.64-1.72 (m, 2H, CH₂CH₂CH₂), 1.38 (s, 9H, C(CH₃)₃). | |
| 25b | 98-100 | δ 9.71 (br s, 1H, phenol OH), 9.30 (br s, 1H, amide NH), 7.17 (dd, J = 2.1/2.1 Hz, 1H, |
| | | aryl 2H), 7.03 (dd, J = 8.1/8.1 Hz, 1H, aryl 5H), 6.92 (d, J = 8.1 Hz, 1H, aryl CH), 6.78- |
| | | 6.86 (m, 1H, carbamate NH), 6.38-6.45 (m, 1H, aryl CH), 2.94 (dt, J = 6.6/6.2 Hz, |
| | | 2H, NHCH₂), 2.26 (t, J = 7.5 Hz, 2H, CH₂(C═O)), 1.66 (tt, J = 7.3/7.3 Hz, 2H, |
| | | CH₂CH₂CH₂), 1.37 (s, 9H, C(CH₃)₃). |
| 25c | 62-65 | δ 7.38 (ddd, J = 7.6/7.6/2.0 Hz, 2H, aryl 3-H and 5-H), 7.23 (ddd, J = 7.6/7.6/1.0 Hz, |
| | | 1H, aryl 4-H), 7.08 (dd, J = 8.5/1.4 Hz, 2H, aryl 2-H and 6-H), 4.67 (br s, 1H, NH), 3.26 |
| | | (dt, J = 7.0/6.2 Hz, 2H, NHCH₂), 2.61 (t, J = 7.0 Hz, 2H, CH₂C═O), 1.94 (tt, J = 7.0/7.0 |
| | | Hz, 2H, CH₂CH₂CH₂), 1.45 (s, 9H, (CH₃)₃). |

### 3-(Phenylcarbamoyl)propylammonium trifluoroacetate (26)

25 (653 mg, 2.35 mmol) was dissolved in TFA/DCM (20 mL 1:1) and stirred for 2 hours at rt. Removal of volatiles under reduced pressure gave 773 mg of semi-solid requiring no further purification.

### 4-amino-N-(2-hydroxyphenyl)butanamide hydrochloride (26a) and 4-amino-N-(3-hydroxyphenyl)butanamide hydrochloride (26b)

Refer to general procedure for synthesis of phenyl substituted N-(2-aminoethyl)-2-(hydroxyphenyl)acetamide hydrochlorides and N-(4-amino) hydroxyphenylbutanamine hydrochlorides.

### Phenyl 4-aminobutanoate hydrochloride salt (26c)

25c was deprotected in a similar fashion to 21b as described in the method for 22b, to give 1.289 g 26c as a yellow semi-solid.

Table 13 lists the ¹H NMR spectral data for selected compounds from Figure 2:

| Cpd | ¹H NMR (DMSO-d₆) |
|---|---|
| 26 | (DMSO-d₆): δ 10.03 (s, 1H, NH), 7.87 (br s, 3H, NH₃⁺), 7.59 (d, J = 7.6 Hz, 2H, phenyl 2-H |
| | and 6-H), 7.29 (dd, J = 7.6/7.6 Hz, 2H, phenyl 3-H and 5-H), 7.02 (dd, J = 7.4/7.4 Hz, 1H, |
| | phenyl 4-H), 2.80-2.91 (m, 2H, CH₂NH₃⁺), 2.42 (t, J = 7.2 Hz, 2H, COCH₂), 1.85 (tt, J = |
| | 7.3 Hz, 2H, CH₂CH₂CH₂). |
| 26a | δ 9.38 (s, 1H, (C═O)NH), 8.06 (s, 3H, NH₃⁺), 7.72 (dd, J = 8.1/1.2 Hz, 1H, aryl 6H), 6.82- |
| | 6.97 (m, 2H, aryl C—H), 6.74 (ddd, J = 7.5/1.8 Hz, 1H, aryl C—H), 5.75 (s, 1H, OH), 2.69- |
| | 2.89 (m, 2H, NH₃⁺CH₂CH₂CH₂(C═O)), 1.81-1.94 (m, 2H, NH₃⁺CH₂CH₂CH₂(C═O)), 1.16- |
| | 1.32 (m, 2H, NH₃⁺CH₂CH₂CH₂(C═O)). |
| 26b | δ 10.04 (s, 1H, (C═O)NH), 8.07 (s, 3H, NH₃⁺), 7.18 (t, J = 2.0 Hz, 1H, (C═O)NH), 6.94- |
| | 7.09 (m, 2H, aryl 2H, 6H), 6.75-6.88 (m, 1H, aryl 5H), 6.37-6.50 (m, 1H, aryl 4H), 5.75 |
| | (s, 1H, OH), 2.71-2.88 (m, 2H, NH₃⁺CH₂CH₂CH₂(C═O)), 2.41 (t, J = 7.4 Hz, 2H, |
| | NH₃⁺CH₂CH₂CH₂ (C═O)), 1.80-1.92 (m, 2H, NH₃⁺CH₂CH₂CH₂(C═O)). |
| 26c | δ 8.22 (br s, 3H, NH₃⁺), 7.42 (ddd, J = 7.6/7.6/2.0 Hz, 2H, aryl 3-H and 5-H), 7.26 (ddd, J = |
| | 7.6/7.6/1.0 Hz, 1H, aryl 4-H), 7.14 (dd, J = 8.5/1.4 Hz, 2H, aryl 2-H and 6-H), 2.82- |
| | 2.92 (m, 2H, CH₂NH₃⁺), 2.74 (t, J = 7.5 Hz, 2H, CH₂C═O), 1.93 (tt, J = 7.6/7.6 Hz, 2H, |
| | CH₂CH₂CH₂). |

### 3-Phthalimidopropanoic acid (28)

Phthalic anhydride (14.8 g, 0.1 mol) and 27 (8.9 g, 0.1 mol, 1 eq) were heated at 150° C. with stirring under a condenser for 2 hours. After cooling to rt, the crude solid was dispersed in water (150 mL) and collected by filtration (suction) before drying to give 20.7 g of white crystalline solid requiring no further purification.

### 1-(2-Hydroxyphenyl)-3-(2-phthalimidoethyl)urea (29a)

A solution of 28 (2.000 g, 9.12 mmol), DPPA (1.966 mL, 9.12 mmol, 1 eq) and TEA (2.543 ml, 2 eq, 18.25 mmol) in dry toluene (60 mL) was stirred at rt, under a nitrogen atmosphere. After disappearance of starting materials by TLC (approximately 1 hour), the mixture was refluxed to promote conversion to the isocyanate. After evolution of nitrogen gas had ceased, the reaction mixture was split into half (by volume). 2-aminophenol (747 mg, 1.5 eq, 6.84 mmol) was added to one half of the isocyanate solution and stirred under reflux for 16 hours. On cooling to rt a yellow precipitate formed, which was collected by filtration (suction) and washed with EtOAc. On drying, 867 mg of pale yellow solid was obtained requiring no further purification.

### 1-(3-Hydroxyphenyl)-3-(2-phthalimidoethyl)urea (29b)

Isocyanate solution was prepared as described for 29a. To the remaining half portion was added 3-aminophenol (747 mg, 1.5 eq, 6.84 mmol) and stirred under reflux for 16 hours.

After cooling and removal of solvent, the crude residue was dispersed in EtOAc (50 mL) and washed with aqueous 2M HCl (2×30 mL). Concentration of the organic layer gave 1.134 g of pale yellow solid.

### 1-(2-Aminoethyl)-3-(2-hydroxyphenyl)urea hydrochloride (30a)

A solution of 29a (700 mg, 2.13 mmol) and hydrazine monohydrate (232 μl, 4.5 mmol, 2.1 eq) in EtOH (20 mL) was stirred under reflux for 2 hours. After cooling to rt, solvent was removed under reduced pressure. The crude residue was dispersed in EtOAc (30 mL) and washed with aqueous 2M HCl (2×30 mL). The combined aqueous layer were concentrated under reduced pressure to give 296 mg of yellow solid requiring no further purification.

### 1-(2-Aminoethyl)-3-(2-hydroxyphenyl)urea hydrochloride (30b)

Deprotection of 29b (700 mg, 2.13 mmol) was carried out as described for 29a to give 252 mg of yellow solid requiring no further purification.

### 2-(2-Hydroxyethyl)isoindoline-1,3-dione (32)

Phthalic anhydride (12.125 g, 81.86 mmol) and 31 (4.94 mL, 81.86 mmol, 1 eq) were heated to 175° C. with stirring, under a water condenser for 2 hours. On cooling, the crude solid was crushed before collecting by filtration (suction) and washing with water to give 13.010 g of beige crystalline solid.

### 2-Phthalimidoethyl phenylcarbamate (33)

32 (2.000 g, 10.46 mmol) was dissolved in dry DCM (30 mL) under a nitrogen atmosphere. Phenyl isocyanate (1.137 ml, 10.46 mmol, 1 eq) was added and the mixture stirred for 48 hours. Hexanes were added to the mixture until a precipitation of a solid was observed. After collection by filtration (suction), this crude solid was purified by column chromatography (eluent EtOAc/Hexanes 30:70 to 50:50 over 10 column volumes to give 400 mg of white solid.

### 2-Aminoethyl phenylcarbamate hydrochloride (34)

Deprotection of 33 (348 mg, 1.13 mmol) was carried out as described for 29a to give 180 mg of white solid requiring no further purification.

### 4-Amino-2-fluorophenol (36)

35 (1.19 g, 7.57 mmol) was dissolved in methanol (40 mL) and hydrogenated over 10% Pd/C (125 mg), at rt and atmospheric pressure. The suspension was filtered over celite and washed with excess MeOH. Removal of excess solvent under reduced pressure afforded 867 mg of light brown solid.

### 1-(3-Fluoro-4-hydroxyphenyl)-3-(2-phthalimidoethyl)urea (37)

Isocyanate solution was prepared as described for 29a via curtius reaction starting with 28 (1.000 g, 4.56 mmol). To this was added 36 (830 mg, 6.53 mmol, 1.4 eq) and stirred with heating under reflux overnight. After cooling, the formed precipitate was collected by filtration (suction) and washed with EtOAc, which on drying gave 866 mg of solid.

### 1-(2-Aminoethyl)-3-(3-fluoro-4-hydroxyphenyl)urea hydrochloride (38)

Deprotection of 37 (800 mg, 2.33 mmol) was carried out as described for 29a to give 487 mg of white solid requiring no further purification.

### tert-Butyl 2-(3-(4-(benzyloxy)phenyl)ureido)ethylcarbamate (38a)

4-Benzyloxyphenyl isocyanate (14.95 g, 66 mmol) was dissolved in DCM and the solution was cooled to 0° C. (ice bath). 14 (1.1 eq, 73 mmol, 11.7 g) was added drop-wise to the solution. At the end of the addition the ice bath was removed and the solution was allowed to stir at rt overnight. A large excess of PE was added to precipitate the product urea, which was then filtered off and further purified by column chromatography, eluent 50/50 PE/EtOAc.

### tert-Butyl 2-(3-(4-hydroxyphenyl)ureido)ethylcarbamate (38b)

38a (8 g, 21 mmol) was dissolved in THF (250 ml) and a drop of CHCl₃, Pd/C (10% mol⁻¹) in suspension in THF was added. The suspension was degassed under vacuum (2 cycles of evacuation, followed by nitrogen filling) before placing under an atmosphere of H₂. The suspension was stirred at it overnight, before filtering through a celite ped, and concentration of the filtrate under reduced pressure. The crude residue was purified by FCC (eluent PE/DCM/EtOAc 100:0:0 to 0:100:0 over 5.5 min, 0:100:0 for 8 min then 0:90:10 for 9 min).

### tert-Butyl 2-(3-(4-(2-fluoroethoxy)phenyl)ureido)ethylcarbamate (38c)

38b (1.00 g, 3.39 mmol), 2-fluoroethanol (0.239 g, 0.219 mL, 3.72 mmol, 1.1. eq) and PPh₃ (0.977 g, 3.72 mmol, 1.1 eq) were dispersed in DCM (20 mL) and THF (3 mL) with stirring at rt. To this, was added DIAD (0.733 mL, 3.72, mmol, 1.1 eq) in DCM (5 mL) followed by further washings with DCM (5 mL). This was stirred at it for 2 days, and TLC (eluent MeOH/DCM 5:95) and LC-MS analysis indicated reaction progression, but consumption of DIAD/PPh₃. A further 0.5 eq each of 2-fluorethanol, PPh₃, and DIAD were added along with THF (10 mL), however solution was not achieved. Stirring was continued over the weekend. After this time, LC-MS analysis indicated the reaction had progressed further, but was not yet complete, so MeCN (10 mL) was added, along with a further 1 eq each of 2-fluoroethanol, triphenylphosphine and DIAD. Stirring was continued at it overnight, after which starting material had almost disappeared by TLC, and the product spot was much stronger. The reaction was stopped, with removal of all volatiles under reduced pressure, and purified without further workup using FCC (eluent DCM/MeOH 1:99 to 10:90 over 10 CV) to give 780 mg of beige solid (67%). In addition, fractions contained a mixture of product and triphenylphosphine oxide were retained.

### 1-(2-Aminoethyl)-3-(4-(2-fluoroethoxy)phenyl)urea hydrochloride (38d)

38c (750 mg, 2.20 mmol) was dissolved in DCM (10 mL) and MeOH (a few drops) with stirring, before addition of 4M HCl/Dioxane (10 mL). The mixture was stirred for 1 hour at rt, and took on a cloudy appearance. PE was added to complete precipitation, however a biphasic system was formed. All volatiles were removed under reduced pressure to give 675 mg of a pale pink solid (quantitative yield).

Table 14 lists the ¹H NMR spectral data for selected compounds from Figure 2:

| Cpd | m.p | ¹H NMR |
|---|---|---|
| 28 | 151-153 | (DMSO-d₆): δ 12.39 (s, 1H, COOH), 7.81-7.89 (m, 4H, aryl C—H), 3.79 (t, J = 7.3 |
| | | Hz, 2H, phth-NCH₂), 2.60 (t, J = 7.4 Hz, 2H, CH₂COOH). |
| 29a | 219-220 | (DMSO-d₆): δ 9.76 (s, 1H, OH), 7.81-7.90 (m, 4H, phth CH), 7.77 (dd, J = 7.8/1.5 |
| | | Hz, 1H, hydroxyphenyl 6-H), 7.76 (s, 1H, NH(C═O)NHAr), 6.95 (t, J = 6.2, 1H, |
| | | NH(C═O)NHAr), 6.62-6.78 (m, 3H, hydroxyphenyl 3-H, 4-H, 5-H), 3.66 (t, J = |
| | | 5.3 Hz, 2H, phth-NCH₂), 3.35 (dt, J = 6.0/5.8 Hz, 2H, CH₂—NH). |
| 29b | 225-228 | (DMSO-d₆): δ 9.15 (br s, 1H, OH), 8.31 (s, 1H, NH(C═O)NHAr), 7.81-7.88 (m, 4H, |
| | | phth CH), 6.91-6.95 (m, 2H, aryl 2-H, 5-H), 6.63 (dd, J = 8.0/1.0 1H, aryl 6-H), |
| | | 6.26 (dd, J = 7.6/1.7 Hz, 1H, aryl 4-H), 6.20 (t, J = 5.8 Hz, 1H, NH(C═O)NHAr), |
| | | 3.68 (t, J = 5.7 Hz, 2H, phth-NCH₂), 3.34 (dt, J = 5.7/5.7 Hz, 2H, CH₂—NH). |
| 30a | 160-165 | (DMSO-d₆): δ 9.92 (br s, 1H, OH), 8.11 (s, 1H, NH(C═O)NHAr), 7.82 (dd, J = |
| | | 7.9/1.7 Hz, 1H, aryl 6-H), 7.27 (t, J = 5.6 Hz, 1H, NH(C═O)NHAr), 6.84 (dd, J = |
| | | 7.7/1.5 Hz, 1H, aryl 3-H), 6.74 (ddd J = 7.4/7.4/1.7 Hz, 1H, aryl 4-H or 5-H), 6.67 |
| | | (ddd, J = 7.8/7.8/1.5 Hz, 1H, aryl 4-H or 5-H), 3.31 (dt, J = 6.2/5.9 Hz, 2H, CH₂NH), |
| | | 2.86 (t, J = 6.3 Hz, 2H, CH₂NH₃⁺). |
| 30b | 106-109 | (DMSO-d₆): δ 8.94 (s, 1H, NH(C═O)NHAr), 8.05 (br s, 3H, NH₃⁺), 6.96-6.98, (m, |
| | | 2H, aryl 2H, 5-H), 6.78 (m, 1H, aryl 6-H), 6.30 (dd, J = 8.0/2.3/0.7 Hz, 1H aryl 4- |
| | | H), 3.31 (t, J = 6.4 Hz, 2H, CH₂ NH₃⁺), 2.83 (dt, J = 6.0/6.0 Hz, 1H, CH₂NH). |
| 32 | 134-137 | (CDCl₃): δ 7.79-7.87 (2H, m, phth C—H), 7.67-7.76 (2H, m, phth C—H), 3.81- |
| | | 3.94 (m, 4H, 2 x CH₂), 2.40 (br s, 1H, OH). |
| 33 | 109-111 | (CDCl₃): δ 7.81-7.90 (2H, m, phth C—H), 7.68-7.77 (2H, m, phth C—H), 7.33 (d, J = |
| | | 8.1 Hz, 2H, 2-H and 6-H phenyl ring), 7.27 (dd, J = 8.8/8.8 Hz, 2H, 3-H and 5-H |
| | | phenyl ring), 7.04 (dd, J = 7.5/7.5 Hz, 1H, 4-H phenyl ring), 6.69 (br s, 1H, NH), |
| | | 4.42 (t, J = 5.2 Hz, 2H, CH₂O), 4.02 (t, J = 5.5 Hz, 2H, NCH₂). |
| 34 | 176-180 | (DMSO-d₆): δ 9.73 (s, 1H, C═ONH), 8.26 (br s, 3H, NH3⁺), 7.48 (d, J = 7.5 Hz, 2H, |
| | | 2-H and 6-H phenyl ring), 7.28 (dd, J = 7.2/7.2 Hz, 2H, 3-H and 5-H phenyl ring), |
| | | 7.00 (dd, J = 7.2/7.2 Hz, 1H, 4-H, phenyl ring), 4.29 (t, J = 5.6 Hz, 2H, CH₂O), 3.09 |
| | | (dt, J = 5.6/5.2 Hz, 2H, NH₃⁺CH₂). |
| 36 | 169-172 | (DMSO-d₆): δ 8.57 (s, 1H, OH), 6.62 (dd, J = 10.1/8.5 Hz, 1H, 6-H), 6.34 (dd, J = |
| | | 10.7/2.8 Hz, 1H, 3-H), 6.20 (ddd, J = 8.3/2.8/1.1 Hz, 1H, 5-H), 4.68 (s, 2H, NH₂). |
| 37 | 226-227 | (DMSO-d₆): δ 9.26 (br s, 1H, OH), 8.21 (s, 1H, NH(C═O)NHAr), 7.77-7.92 (m, 4H, |
| | | phth C—H), 7.16-7.25 (m, 1H, aryl C—H), 6.69-6.79 (m, 2H, aryl C—H), 6.20 (t, J = |
| | | 5.97 Hz, 1H, NH(C═O)NHAr), 3.67 (t, J = 5.3 Hz, 2H, NCH₂), 3.30-3.35 (m, 2H, CH₂NH). |
| 38 | 190-195 | (DMSO-d₆): δ 9.35 (br s, 1H, OH), 8.93 (s, 1H, NH(C═O)NHAr), 7.98 (br s, 3H, |
| | | NH₃⁺), 7.33-7.42 (m, 1H, aryl C—H), 6.78-6.88 (m, 2H, aryl C—H), 6.55 (t, J = 5.1 |
| | | Hz, 1H, NH(C═O)NHAr), 3.30 (dt, J = 6.1/5.6 Hz, 2H, CH₂NH), 2.79-2.91 (m, 2H, NH₃⁺CH₂). |

### 1-(2-(Cyclopropylmethoxy)ethoxy)-4-(benzyloxy)benzene (39a)

3 (563 mg, 4.85 mmol), triphenylphosphine (1.528 g, 5.82 mmol, 1.2 eq) and 4-(benzyloxy)phenol (1.166 g, 5.82 mmol, 1.2 eq) were dissolved in dry DCM (10 mL) and stirred in a flame-dried flask under nitrogen atmosphere. DEAD (0.917 mL, 5.82 mmol, 1.2 eq) was diluted in dry DCM (10 mL) before dropwise addition to the reaction mixture at rt. The resulting solution was stirred overnight at rt under a nitrogen atmosphere. Approximately half the solvent was removed and the resulting slurry dissolved in hexanes (100 mL) and washed with aqueous 0.5 M NaOH (2×50 mL), water (3×50 mL) and brine (1×50 mL). The remaining solvent was removed and the product purified by flash column chromatography (eluent EtOAc/hexanes 15:85) to give 842 mg of white waxy solid.

### 1-(2-(4-Fluorophenethyloxy)ethyloxy)-4-(benzyloxy)benzene (39b)

6 (1.4273 g, 7.78 mmol), triphenylphosphine (2.448 g, 9.33 mmol, 1.2 eq), and 4-(benzyloxy)phenol (1.558 g, 7.78 mmol, 1 eq) were dissolved in dry DCM (30 mL) and stirred in a flame-dried flask under nitrogen atmosphere. Di-tert-butyl azodicarboxylate (2.149 g, 9.33 mmol, 1.2 eq) was dissolved in dry DCM (10 mL) and added dropwise over 5 minutes. The mixture was stirred for 4 h at rt under a nitrogen atmosphere. After removal of half the solvent, the resulting slurry was diluted with hexanes (30 mL) and washed with aqueous 2 M HCl (2×30 mL), aqueous 2 M NaOH (2×30 mL), water (2×30 mL) and brine (1×30 mL). The organic layer was concentrated and purified by flash column chromatography (eluent EtOAc/hexanes 15:85) to give 999 mg of white crystalline solid.

### 1-(2-(Cyclopentyloxy)ethoxy)-4-(benzyloxy)benzene (39c)

8 (3.751 g, 28.81 mmol), triphenylphosphine (9.448 g, 36.02 mmol, 1.25 eq), and 4-(benzyloxy)phenol (5.769 g, 28.81 mmol, 1 eq) were dissolved in DCM (70 mL). Di-tert-butyl azodicarboxylate (8.294 g, 36.02 mmol, 1.25 eq) in DCM (20 mL) was added dropwise to the reaction mixture and allowed to stir overnight. After removal of approximately half of the solvent from the reaction mixture, the reulsting slurry was diluted with hexanes (100 mL) and washed with aqueous 1 M HCl (2×50 mL), aqueous 1 M NaOH (2×50 mL), water (2×50 mL) and brine (1×50 mL). The organic layer was concentrated and redissolved in DCM (30 mL) before addition of hexanes a precipitate of triphenylphosphine oxide began to form. The flask was left in the freezer for 1 h before filtration of the precipitate and washing with hexanes and Et₂O. After concentration of the filtrate, purification was achieved via column chromatography (eluent Et₂O/hexanes 10:90) to give 6.75 g of clear colourless oil.

### 1-((4-(2-Ethoxyethoxy)phenoxy)methyl)benzene (39d)

9 (901 mg, 10.00 mmol), triphenylphosphine (3.147 g, 12.00 mmol, 1.2 eq) and 4-(benzyloxy)phenol (2.403 g, 12.00 mmol, 1.2 eq) were dissolved in dry DCM (40 mL) under nitrogen atmosphere with stirring until complete solution was achieved. DEAD (1.89 mL, 12.00 mmol, 1.2 eq) was diluted in dry DCM (10 mL) before dropwise addition to the reaction mixture at rt. The resulting solution was stirred for 48 h at rt. Approximately half the solvent was removed and the resulting brown slurry dissolved in hexanes (150 mL) and washed with aqueous 2 M NaOH solution (2×70 mL), water (3×70 mL) and brine (1×70 mL). The remaining solvent was evaporated and the product purified by flash column chromatography (eluent EtOAc/hexanes 20:80) to give 2.324 g of white crystalline solid.

### 1-((4-(2-Isopropyloxyethoxy)phenoxy)methyl)benzene (39e)

9a (21 g, 0.2 mol), triphenylphosphine (68.2 g, 0.26 mol, 1.28 eq), and 4-(benzyloxy)phenol (40 g, 0.2 mol, 1 eq) were dissolved in DCM (500 mL). DIAD (51.5 mL, 0.26 mol, 1.28 eq) in DCM (200 mL) was added dropwise to the reaction mixture and allowed to stir overnight. Reaction monitored by TLC in diethyl ether/petroleum. Ether 40-60 (PE) (3:7) showed presence of some starting material. Stirring was continued for further 3 h after addition of further DIAD (21.0 mL, 0.05 mole). After removal of approximately half of the solvent from the reaction mixture, the resulting slurry was diluted with PE (500 mL). Triphenylphosphine oxide precipitate. was filtered and filtrate was washed with aqueous 1 M HCl (2×250 mL), aqueous 1 M NaOH (3×250 mL), water (2×250 mL) and brine (1×300 mL). The organic layer was concentrated and re-dissolved in diethyl ether (150 mL). On addition of PE (approx 300 a precipitate of triphenylphosphine oxide began to form. The flask was left in the freezer for 1 hour before filtration of the precipitate and washing with pet ether. After concentration of the filtrate, purification was achieved via column chromatography using gradient solvent systems (500 mL-10% ether in PE followed by 20% ether in PE) to give 48.3 g (84%) of the desired product.

### 4-(2-(Cyclopropylmethoxy)ethoxy)phenol (40a)

39a (840 mg, 2.82 mmol) was dissolved in EtOH (40 mL) before hydrogenating over 10% Pd/C (119 mg) at rt and atmospheric pressure for 4 h. The suspension was filtered over celite and washed with excess EtOH. Excess solvent was removed to give amber oil. The crude oil was purified by flash column chromatography (eluent EtOAc/hexanes 30/70) to give 508 mg colourless oil.

### 4-(2-(4-Fluorophenethyloxy)ethyloxy)phenol (40b)

39b was hydrogenated according to the method for 40a. After filtration over celite and evaporation of volatiles, no further workup was required and the desired compound was isolated in quantitative yield as clear oil.

### 4-(2-(Cyclopentyloxy)ethoxy)phenol (40c)

39c was hydrogenated according to the method for 40a. After addition of powdered charcoal and filtration over celite, no workup was required and the desired compound isolated in quantitative yield as clear oil.

### 4-(2-Ethoxyethoxy)phenol (40d)

39d (904 mg, 3.32 mmol) was dissolved in EtOH (60 mL) before hydrogenating over 10% Pd/C (168 mg) at rt and atmospheric pressure for 48 h. The suspension was filtered over celite and washed with excess EtOH. Removal of excess solvent gave a viscous amber oil. The crude oil was dissolved in DCM (20 mL) and washed with aqueous 2 M NaOH solution (3×20 mL). The combined aqueous extracts were acidified with concentrated HCl (until the pH was below 7) to effect an emulsion, before extracting with DCM (3×30 mL). The combined organic layers were washed with water (1×30 mL) and brine (1×30 mL). Solvent removal afforded 413 mg of clear, colourless oil.

### 4-(2-Isopropyloxyethoxy)phenol (40e)

To a stirred solution of 39e (48 g, 17 mmol) in THF (500 mL), was added 10% Pd/C (2.5 g) and the solution was stirred at rt for 8 h under hydrogen gas (balloon). Reaction was monitored by TLC in PE: Ethyl acetate (6:4). To this solution again added 10% Pd/C (1.5 g) and the mixture was stirred further for 8 h. Mixture was then passed through celite bed and filtrate was concentrated to obtain desired phenol (33 g, 99%).

### 4-(2-Propoxyethoxy)phenol (40f)

8c was hydrogenated in THF in a similar manner to the procedure described for the synthesis of 40e, to give 1.051 g (100%) of a clear colourless oil.

### 2-((4-(2-(Cyclopropylmethoxy)ethoxy)phenoxy)methyl)oxirane (41a)

40a (450 mg, 2.16 mmol) was dissolved in aqueous 2 M NaOH solution (1.5 mL) and stirred for 10 minutes. Epichlorohydrin (507 μL, 6.481 mmol, 3 eq) was added and the mixture stirred at 60° C. for 24 h. The cooled mixture was extracted with DCM (3×25 mL) and the organic layers combined. After solvent removal, the product purified by flash column chromatography (eluent EtOAc/hexanes 30:70) to give 356 mg of colourless oil.

### 2-((4-(2-(4-Fluorophenethyloxy)ethyloxy)phenoxy)methyl)oxirane (41b)

NaH 60% suspension in mineral oil (13 mg, equivalent to 7.8 mg of NaH, 0.33 mmol, 1.1 eq) was suspended in dry DMF (2 mL) with stirring, under a nitrogen atmosphere. To this was added 40b (82 mg, 0.30 mmol) in dry DMF (4 mL) and stirred until no further hydrogen gas evolution was visible. Epichlorohydrin (800 μL, 10.22 mmol, 34 eq) was added and the reaction stirred overnight at rt. The reaction mixture was diluted with water (30 mL) before extraction with Et₂O (3×30 mL). The combined organic extracts were concentrated before purification over a silica plug (initial wash with hexanes, followed by EtOH/DCM 5:95) to give 70 mg of clear yellow oil.

### 2-((4-(2-(Cyclopentyloxy)ethoxy)phenoxy)methyl)oxirane (41c)

NaH 60% suspension in mineral oil (863 mg, equivalent to 518 mg of NaH, 21.58 mmol, 1.1 eq) was suspended in dry DMF (20 mL) with stirring under a nitrogen atmosphere. After 5 minutes 40c (4.360 g, 19.61 mmol) in dry DMF (20 mL) was added dropwise with the vessel cooled over an ice bath. This was then allowed to stir at it for 20 minutes before addition of epichlorohydrin (15.34 mL, 196.10 mmol, 10 eq). The mixture was stirred for 7 h then quenched cautiously with MeOH. After removal of all volatiles, the crude residue was partitioned between water (30 mL) and Et₂O (30 mL) and the aqueous layer washed again with Et₂O (3×30 mL). The combined organic extracts were concentrated before purification over a silica plug (initial wash with hexanes, followed by EtOH/DCM 5:95) to give 4.558 g of clear yellow oil.

### 2-((4-(2-Ethoxyethoxy)phenoxy)methyl)oxirane (41d)

40d (413 mg, 2.27 mmol) was dissolved in aqueous 2 M NaOH solution (4.0 mL) and stirred for 10 minutes. Epichlorohydrin (533 μL, 6.81 mmol, 3 eq) was added and the mixture stirred at 60° C. for 24 h. The cooled mixture was extracted with DCM (3×20 mL) and the organic layers combined. After solvent removal, the product purified by flash column chromatography (eluent Et₂O) to give 417 mg of colourless oil.

### 2-((4-(2-Isopropoxyethoxy)phenoxy)methyl)oxirane (41e)

40e (32.5 g, 0.16 mol) and sodium hydroxide (1.2 equiv.; 8.0 g, 0.2 mol) were dissolved in water (200 ml). Mixture (˜pH 14) was heated to 40° C. and was stirred for 30 min with stirring at 400 rpm. It was then cooled to RT and was added to epichlorohydrin (2.5 equiv., 55 mL, 0.68 mol) in portions over the period of 45 min at 40° C. The reaction was isothermally continued at 60° C. for another 24 h. Completion of reaction was monitored by LCMS. Desired compound was purified by column chromatography. Yield=12.5 g, 92% (effective). Starting material recovered was 22 g. Theoretical Yield (effective): 13.5 g

### 2-((4-(2-Propoxyethoxy)phenoxy)methyl)oxirane (41f)

40f (1.09 g, 5.55 mmol), NaOH (233 mg, 5.83 mmol, 1.05 eq) and epichlorhydrin (10 mL) were placed in a 30 mL MW vial. The mixture was heated at 120° C. in the MW reactor on a dynamic program (maximum pressure 250 psi, maximum power 300W) for 30 minutes. After cooling the reaction mixture was diluted with water (50 mL) and extracted with DCM (3×25 mL). The combined organic layers were washed with water (50 mL) before concentration. The crude product was purified by FCC (eluent EtOAc/PE 3:97 to 60:40 over 10 column volumes) to give 1.202 g of a clear colourless oil

### 2-((4-(2-Cyclopropoxyethoxy)phenoxy)methyl)oxirane (41g)

8g was alkylated with epichlorohydrin in a similar fashion to the procedure described for the synthesis of 41f.

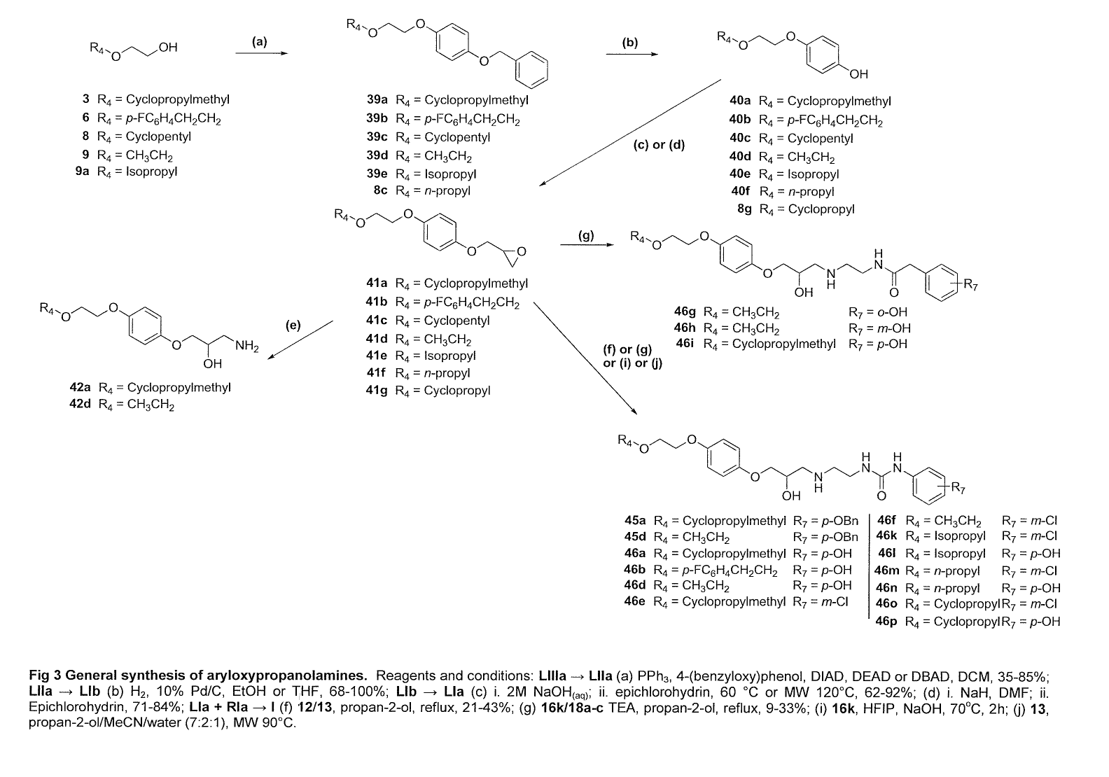

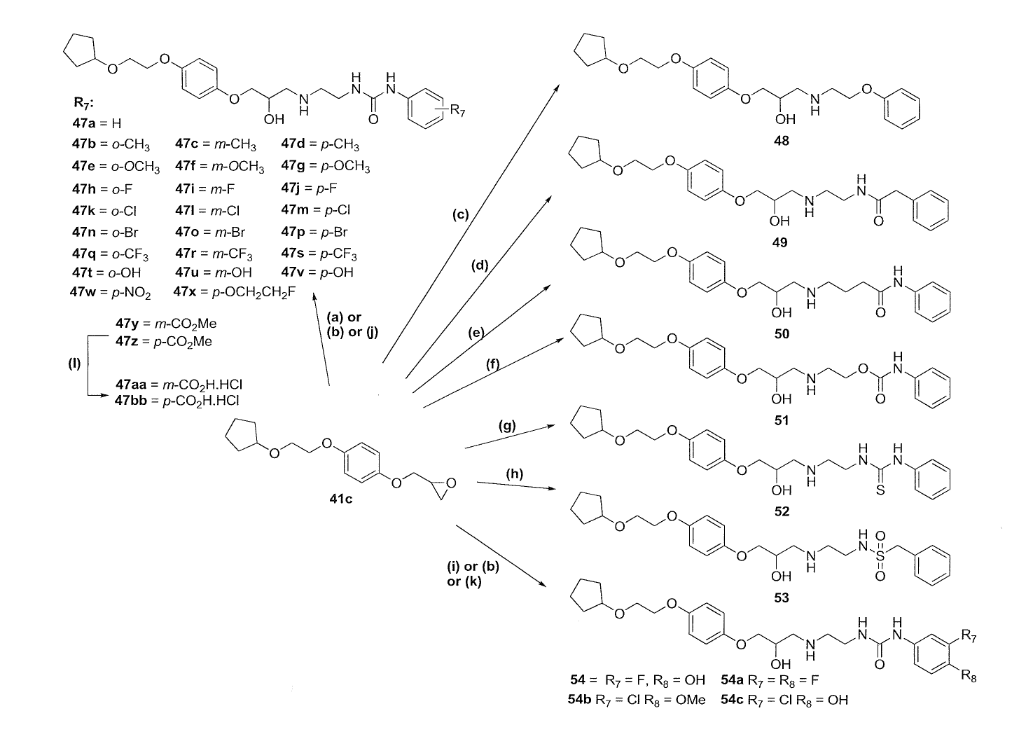

Table 15 lists the ¹H NMR spectral data for selected compounds from Figure 3:

| | m.p; | |
|---|---|---|
| Cpd | HPLC | ¹H NMR |
| 39a | 30.5-32.5 | δ 7.33-7.45 (m, 5H, aromatic benzyl CH), 6.95, 6.91 (d, J = 9.2 Hz, 2 x 2H, |
| | | para-disubstituted aryl ring), 5.01 (s, 2H, PhCH₂O), 4.10 (t, J = 5.0 Hz, 2H, |
| | | CH₂OArOBz), 3.82 (t, J = 5.0 Hz, 2H, CH₂CH₂OArOBz) 3.42 (d, J = 6.8 Hz, 2H, |
| | | ^{c}PrCH₂O) 1.10-1.20 (m, 1H, CH), 0.54-0.66 (m, 2H, ^{c}Pr CH₂)*, 0.23-0.34 |
| | | (m, 2H, ^{c}Pr CH₂)*. *Refers to cis-protons of ^{c}Pr ring. |
| 39b | 59-61 | δ 7.30-7.45 (m, 5H, aromatic benzyl CH), 7.19 (dd, J = 8.6/5.5 Hz, 2H, 3-H |
| | | and 5-H of fluorophenyl ring), 6.97 (dd, J = 8.8/8.8 Hz, 2H, 2-H and 6-H of |
| | | flurophenyl ring), 6.91, 6.85 (d, J = 9.2 Hz, 2 x 2H, para-disubstituted aryl-dioxy |
| | | ring), 5.02 (s, 2H, PhCH₂O), 4.06 (t, J = 4.7 Hz, 2H, CH₂OArOBz), 3.78 (t, J = |
| | | 4.7 Hz, 2H, CH₂CH₂OArOBz), 3.72 (t, J = 7.1 Hz, 2H, FC₆H₄CH₂CH₂O), (t, J = |
| | | 7.1 Hz, 2H, FC₆H₄CH₂) |
| 39c | δ 7.34-7.48 (m, 5H, aromatic benzyl CH), 6.91, 6.96 (d, J = 9.2 Hz, 2 x 2H, aryl-dioxy ring), | |
| | 5.04 (s, 2H, PhCH₂O), 4.09 (t, J = 4.9 Hz, 2H, CH₂OArOBz), 4.02-4.06 (m, 1H, ^{c}Pe CH), | |
| | 3.76 (t, J = 5.3 Hz, 2H, CH₂CH₂OArOBz), 1.73-1.84 (m, 6H, ^{c}Pe CH₂), 1.56-1.63 (m, 2H, ^{c}Pe CH₂). | |
| 39d | 32.5-34.5° C. | δ 7.25-7.32 (m, 5H, aromatic benzyl CH), 6.90, 6.86 (d, J = 9.2 Hz, 2 x 2H, |
| | (lit: 35- | para-disubstituted aryl ring), 5.01 (s, 2H, PhCH₂O), 4.07 (t, J = 4.9 Hz, 2H, |
| | 37° C.)³³. | CH₂OArOBz), 3.77 (t, J = 4.9 Hz, 2H, CH₂CH₂OArOBz), 3.60 (q, J = 7.0 Hz, 2H, |
| | | CH₃CH₂O), 1.24 (t, J = 7.0 Hz, 3H CH₃). |

| | |
|---|---|
| 40a | δ 6.72-6.77 (m, 4H, aryl-H), 5.70-5.50 (br s, 1H, OH), 4.06 (t, J = 5.0 Hz, 2H CH₂OAr), |
| | 3.81 (t, J = 5.0 Hz, 2H, CH₂CH₂OAr), 3.40 (d, J = 6.8 Hz, 2H, ^{c}PrCH₂O), 1.07-1.11 (m, 1H, |
| | ^{c}Pr CH), 0.48-0.60 (m, 2H, ^{c}Pr CH₂)*, 0.17-0.28 (m, 2H, ^{c}Pr CH₂)*. *Refers to cis-protons |
| | of ^{c}Pr ring. |
| 40b | δ 7.18 (dd, J = 8.6/5.5 Hz, 2H, 3-H and 5-H of fluorophenyl ring), 6.96 (dd, J = 8.8/8.8 Hz, 2H, |
| | 2-H and 6-H of flurophenyl ring), 6.74, 6.76 (d, J = 9.2 Hz, 2 x 2H, para-disubstituted phenol), |
| | 6.00 (br s, 1H, OH), 4.05 (t, J = 4.7 Hz, 2H, CH₂OAr), 3.80 (t, J = 4.9 Hz, 2H, CH₂CH₂OAr), |
| | 3.75 (t, J = 7.2 Hz, 2H, FC₆H₄CH₂CH₂O), 2.90 (t, J = 7.1 Hz, 2H, FC₆H₄CH₂). |
| 40c | δ 7.60 (br s, 1H, OH), 6.69, 6.73 (d, J = 9.2 Hz, 2 x 2H, para-disubstituted phenol), 3.96- |
| | 3.99 (m, 3H, CH, CH₂OAr), 3.70 (t, J = 5.0 Hz, 2H, ^{c}PeOCH₂), 1.62-1.78 (m, 6H, ^{c}Pe CH₂), |
| | 1.45-1.53 (m, 2H, ^{c}Pe CH₂). |
| 40d | δ 6.81, 6.74 (d, J = 9.0 Hz, 2H, aryl-H), 4.57 (br s, 1H OH); 4.06 (t, J = 4.9 Hz, 2H, CH₂OAr), |
| | 3.77 (t, J = 4.9 Hz, 2H, CH₂CH₂OAr), 3.60 (q, J = 7.0 Hz, 2H, CH₃CH₂O), 1.24 (t, J = 7.0 Hz, |
| | 3H CH₃).¹⁵ |
| 41a | δ 6.83 (s, 4H, aryl CH), 4.15 (dd, J = 11.0/3.2 Hz, 1H, ArOCH₂CH), 4.06 (t, 4.9 Hz, 2H, |
| | CH₂OAr), 3.88 (dd, J = 11.0/5.7 Hz, 1H, ArOCH₂CH), 3.78 (t, J = 5.0 Hz, 2H, CH₂CH₂OAr), |
| | 3.36 (d, J = 6.8 Hz, 2H, ^{c}PrCH₂O), 3.29-3.33 (m, 1H, epoxide CH), 2.87 (dd, J = 4.8/4.3 Hz, |
| | 1H, epoxide CH₂), 2.72 (dd, J = 4.9/2.7 Hz, 1H, epoxide CH₂), 1.05-1.10 (m, 1H, ^{c}Pr CH), |
| | 0.51-0.55 (m, 2H, ^{c}Pr CH₂)*, 0.19-0.22 (m, 2H, ^{c}Pr CH₂)*. *Refers to cis-protons of ^{c}Pr |
| | ring. |
| 41b | δ 7.19 (dd, J = 8.6/5.6 Hz, 2H, 3-H and 5-H of fluorophenyl ring), 6.96 (dd, J = 8.8/8.8 Hz, 2H, |
| | 2-H and 6-H of flurophenyl ring), 6.85 (s, 4H, aryl-dioxy ring), 4.17 (dd, J = 11.1/3.2 Hz, 1H, |
| | ArOCH₂CH), 4.06 (t, J = 4.7 Hz, 2H, CH₂OAr), 3.90 (dd, J = 11.0/5.7 Hz, 1H, ArOCH₂CH), |
| | 3.78 (t, J = 4.9 Hz, 2H, CH₂CH₂OAr), 3.72 (t, J = 7.1 Hz, 2H, FC₆H₄CH₂CH₂O), 3.32-3.35 |
| | (m, 1H, epoxide CH), 2.89 (t, J = 7.1 Hz, 2H, FC₆H₄CH₂), 2.87-2.91 (m, 1H, epoxide CH₂), |
| | 2.74 (dd, J = 4.9/2.7 Hz, 1H, epoxide CH₂). |
| 41c | δ 6.80 (s, 4H, aryl C—H), 4.11 (dd, J = 11.1/3.1 Hz, 1H, ArOCH₂CH), 3.99 (t, J = 4.9 Hz, 2H, |
| | CH₂OAr), 3.92-3.96 (m, 1H, ^{c}Pe CH), 3.82 (dd, J = 11.1/5.7, 1H, ArOCH₂CH), 3.67 (t, J = |
| | 5.3 Hz, 2H, ^{c}PeOCH₂), 3.25-3.29 (m, 1H, epoxide CH), 2.82 (d, J = 4.9/4.9 Hz, 1H, epoxide |
| | CH₂), 2.67 (dd, J = 5.0/2.7 Hz, 1H, epoxide CH₂), 1.58-1.77 (m, 6H, ^{c}Pe CH₂), 1.41-1.56 |
| | (m, 2H, ^{c}Pe CH₂). |
| 41d | δ 6.83-6.88 (m, 4H, para-disubstituted aryl ring), 4.17 (dd, J = 11.2/3.2 Hz, 1H, ArOCH₂CH), |
| | 4.07 (t, J = 4.9 Hz, 2H, CH₂OAr), 3.91 (dd, J = 10.08/5.6 Hz), 1H, ArOCH₂CH), 3.77 (t, J = 4.9 |
| | Hz, 2H, CH₂CH₂OAr), 3.60 (q, J = 7.0 Hz, 2H, CH₃CH₂O), 3.34 (m, 1H, epoxide CH), 2.89 |
| | (dd, J = 5.0/4.0 Hz, 1H, epoxide CH₂), 2.74 (dd, J = 5.0/2.6 Hz, 1H, epoxide CH₂), 1.25 (t, J = |
| | 7.0 Hz, 3H, CH₃). |

### Compounds of Formula I

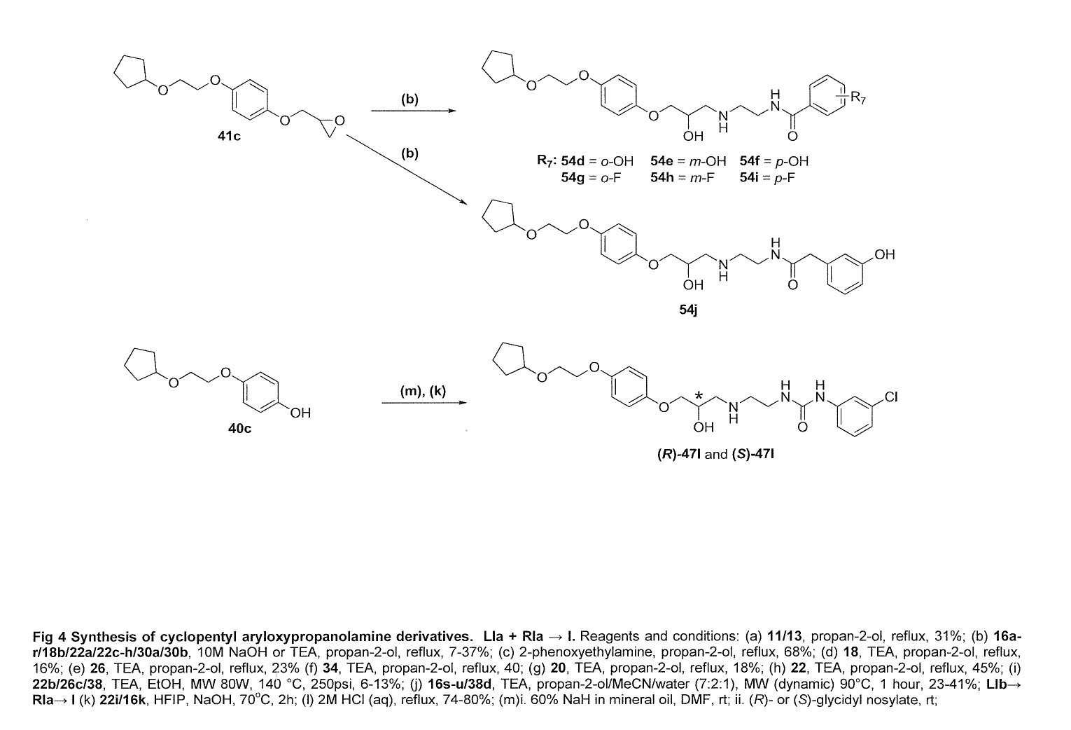

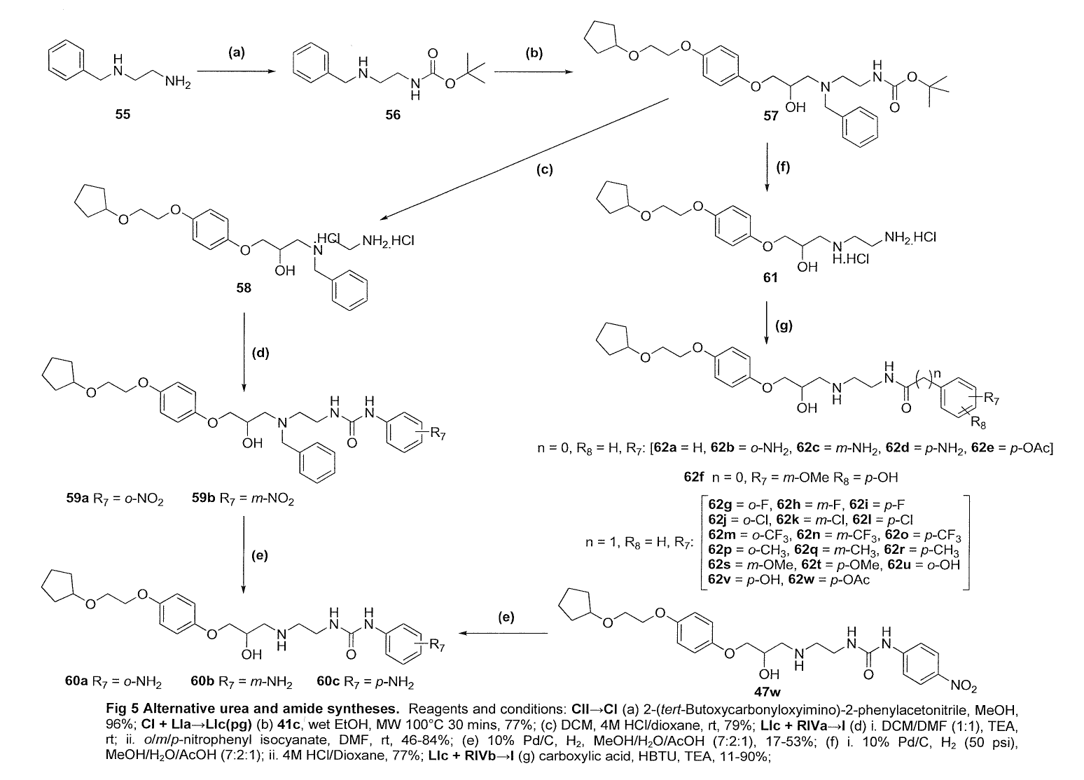

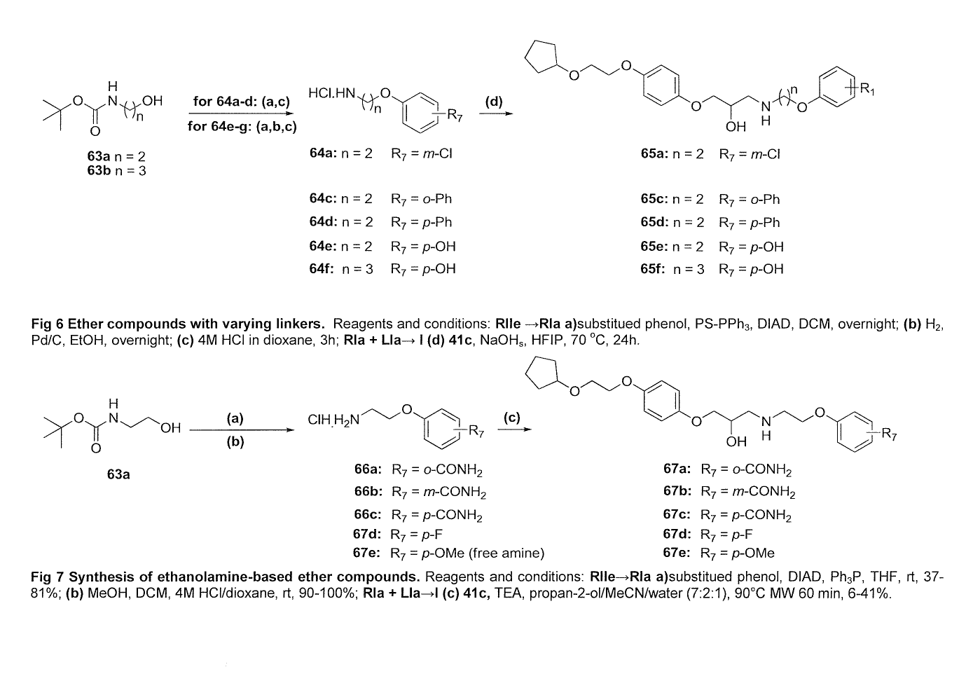

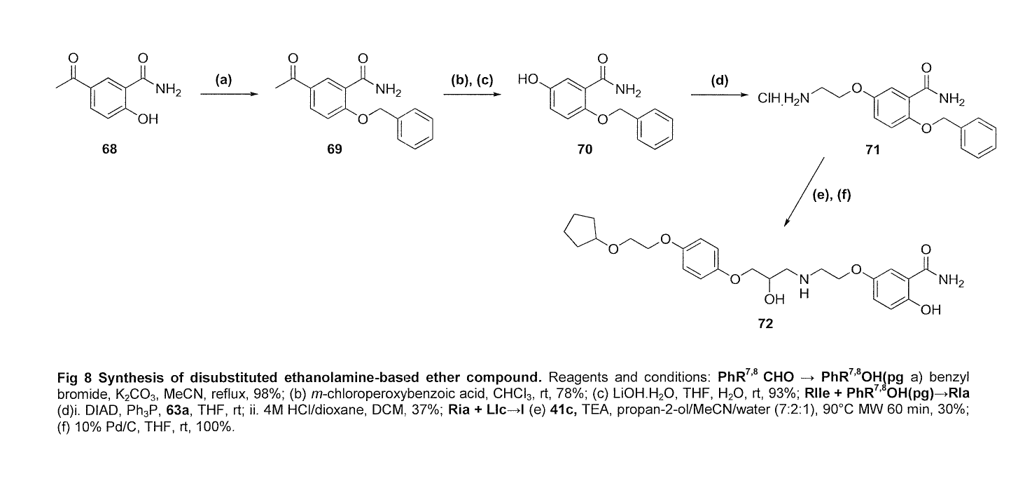

### General Procedure for Synthesis of Substituted 1-(2-(3-(4-(2-(alkyloxy)ethoxy)phenoxy)-2-hydroxypropylamino)ethyl)-3-(aryl)ureas (45a/d, 46a/b/d, 47a-s/v)—FIGS. 3 and 4

Substituted 1-(2-aminoethyl)-3-(aryl)urea (1 eq, compounds 11, 12, 13 or 16a-r) and epoxide (50 mg, compounds 41a-d) were suspended in propan-2-ol (3 mL). In the case where 1-(2-aminoethyl)-3-(aryl)ureas were hydrochloride salts, NaOH (1.1 eq as 10 M aqueous solution) was also added. The mixture was heated under reflux overnight, after which all solvent was removed under vacuum. The crude residue was purified via PLC (eluent NH₃/MeOH/DCM 2:10:88). Analogues with substitution meta to the urea group were purified using a weaker eluent (NH₃/MeOH/DCM 2:5:93). The final aryloxypropanolamines were freeze-dried to give white solids.

### 1-(2-(3-(4-(2-(Cyclopropylmethoxy)ethoxy)phenoxy)-2-hydroxypropylamino)ethyl)-3-(3-chlorophenyl)urea hydroformate (46e)

41a was opened with 16k according to the method described for 47t. Purification via PLC (eluent NH₃/MeOH/DCM 1:10:89) and preparative HPLC afforded 9 mg of beige semi-solid.

### 1-(2-(3-(4-(2-Ethoxyethoxy)phenoxy)-2-hydroxypropylamino)ethyl)-3-(3-chlorophenyl)urea hydroformate (46f)

41d was opened with 16k according to the method described for 47t. Purification via PLC (eluent NH₃/MeOH/DCM 1:10:89) and preparative HPLC afforded 11 mg of beige semi-solid.

### N-(2-(3-(4-(2-ethoxyethoxy)phenoxy)-2-hydroxypropylamino)ethyl)-2-(2-hydroxyphenyl)acetamide (46g)

41d was opened with 18a according to the method described for 47t. Purification via PLC (eluent NH₃/MeOH/DCM 1:7.5:91.5) and preparative HPLC afforded 22 mg of colourless semi-solid.

### N-(2-(3-(4-(2-ethoxyethoxy)phenoxy)-2-hydroxypropylamino)ethyl)-2-(3-hydroxyphenyl)acetamide (46h)

41d was opened with 18b according to the method described for 47t. Purification via PLC (eluent NH₃/MeOH/DCM 1:10:89) and preparative HPLC afforded 30 mg of colourless semi-solid.

### N-(2-(3-(4-(2-(cyclopropylmethoxy)ethoxy)phenoxy)-2 hydroxypropylamino)ethyl)-2-(4-hydroxyphenyl)acetamide (46i)

41a was opened with 18c according to the method described for 47t. Purification via PLC (eluent NH₃/MeOH/DCM 1:7.5:91.5) and preparative HPLC afforded 17 mg of colourless semi-solid.

### General Method for Epoxide Openings in HFIP

Epoxide (0.5 to 1 mmol) and amine salt (1.3 to 2 eq) are dissolved in HFIP 6 ml at rt, and NaOH (3.75 eq) is added portion wise. Heat the solution at 70° C. Reaction followed by HPLC. After disappearance of epoxide, stop reaction and wet load on silica (hand packed Flashmaster cartridge: 10 g/70 ml) 35 ml/min. gradient: DCM/1M ammonia in MeOH, 2 minutes at 99:1, 8 min gradient to 90:10 then plateau for 7 min at this concentration, then 8 min gradient to 80:20 then plateau for 10 min. Fraction Analysis by LC/MS to identify product fractions.

### 1-(2-(3-(4-(2-(Isopropyloxy)ethoxy)phenoxy)-2-hydroxypropylamino)ethyl)-3-(3-chlorophenyl)urea (46k)

16k and 41e were reacted as described in the general method for epoxide openings in HFIP. After 24 h no reaction observed by HPLC, so 0.5 eq. NaOH added and carried on 4 days at 70° C. LCMS showed some product. Reaction stopped and purified by Flash master as described.

### General Procedure for Epoxide Openings in propan-2-ol/MeCN/water (7:2:1)

Epoxide (100 mg) and amine (2 eq) were placed in a 10 mL MW vial. In the case of amine salts, TEA (2.1 eq) are added, and a solvent mixture consisting of propan-2-ol/MeCN/water (7:2:1) (3-5 mL) is added. The mixture is heated in the MW reactor for 55-60 minutes at 90° C. on a dynamic program (maximum pressure 250 psi, maximum power 300W). The reaction mixture is concentrated and purified by FCC (eluent 1M NH₃ in MeOH/DCM 0:100 to 20:80).

### 1-(2-(3-(4-(2-(Isopropyloxy)ethoxy)phenoxy)-2-hydroxypropylamino)ethyl)-3-(4-hydroxyphenyl)urea (461)

13 and 41e were reacted according to the general procedure for epoxide openings in propan-2-ol/MeCN/water (7:2:1) (Yield 22%).

### 1-(2-(3-(4-(2-(Propyloxy)ethoxy)phenoxy)-2-hydroxypropylamino)ethyl)-3-(3-chlorophenyl)urea (46m)

16k and 41f were reacted according to the general procedure for epoxide openings in propan-2-ol/MeCN/water (7:2:1) (Yield 27%, white amorphous solid).

### 1-(2-(3-(4-(2-(Propyloxy)ethoxy)phenoxy)-2-hydroxypropylamino)ethyl)-3-(4-hydroxyphenyl)urea (46n)

13 and 41f were reacted according to the general procedure for epoxide openings in propan-2-ol/MeCN/water (7:2:1) (Yield 11%, off-white amorphous solid).

### 1-(3-Chlorophenyl)-3-(2-(3-(4-(2-cyclopropoxyethoxy)phenoxy)-2-hydroxypropylamino)ethyl)urea (46o)

16k and 41g were reacted as described in the general method for epoxide openings in HFIP.

1-(2-(3-(4-(2-Cyclopropoxyethoxy)phenoxy)-2-hydroxypropylamino)ethyl)-3-(4-hydroxyphenyl)urea (46p)

13 and 41g were reacted as described in the general method for epoxide openings in HFIP.

Table 16 lists the ¹H NMR spectral data for selected compounds from Figure 3:

| | m.p; | |
|---|---|---|
| Cpd | HPLC | ¹H NMR |
| 45a | 131-133° C. | (DMSO-d₆): δ 8.41 (s, 1H, NH(C═O)NHAr), 7.30-7.46 (m, 5H, benzyl C—H), |
| | Rₜ: 4.57 | 7.27 (d, J = 9.0 Hz, 2H, 2-H and 6-H of ureidoaryloxy ring), 6.88 (d, J = 9.0 Hz, |
| | (S 1a), | 2H, 3-H and 5-H of ureidoaryloxy ring), 6.84 (s, 4H, C—H aryl-dioxy ring), 6.11 |
| | 14.17 (S | (t, J = 5.3 Hz, 1H, NH(C═O)NHAr), 5.02 (s, 2H, benzyl CH₂), 3.99 (t, J = 4.5 |
| | 3). | Hz, 2H, CH₂OAr), 3.77-3.92 (m, 3H, CH(OH), ArOCH₂), 3.67 (t, J = 4.7 Hz, |
| | | 2H, OCH₂CH₂OAr), 3.28 (d, J = 6.8 Hz, 2H, ^{c}PrCH₂O), 3.12-3.21 (m, 2H, |
| | | NHCH₂CH₂), 2.58-2.78 (m, 4H, CH(OH)CH₂NH, NHCH₂CH₂), 0.94-1.05 |
| | | (m, 1H, ^{c}Pr CH), 0.42-0.49 (m, 2H, ^{c}Pr CH₂)*, 0.13-0.19 (m, 2H, ^{c}Pr CH₂)*. |
| | | *Refers to cis-protons of ^{c}Pr ring. |
| 45d | Rₜ: 4.14 | 143-145 (DMSO-d₆): δ 8.44 (s, 1H, NH(C═O)NHAr), 7.30-7.45 (m, 5H, |
| | (S 1a), | benzyl C—H), 7.28 (d, J = 9.0 Hz, 2H, 2-H and 6-H of ureidoaryloxy ring), 6.88 |
| | 13.25 (S | (d, J = 9.0 Hz, 2H, 3-H and 5-H of ureidoaryloxy ring), 6.84 (s, 4H, C—H aryl- |
| | 3). | dioxy ring), 6.14 (t, J = 5.2 Hz, 1H, NH(C═O)NHAr), 5.02 (s, 2H, benzyl CH₂), |
| | | 3.99 (t, J = 4.5 Hz, 2H, CH₂OAr), 3.79-3.91 (m, 3H, CH(OH), ArOCH₂), 3.65 |
| | | (t, J = 4.7 Hz, 2H, OCH₂CH₂OAr), 3.48 (q, J = 7.0 Hz, 2H, CH₃CH₂), 3.16- |
| | | 3.20 (m, 2H, NHCH₂CH₂), 2.60-2.80 (m, 4H, CH(OH)CH₂NH, NHCH₂CH₂), |
| | | 1.12 (t, J = 7.0 Hz, 3H, CH₃). |
| 46a | semi- | (MeOD-d₄): δ 7.09-7.16 (m, 2H, aryl C—H ortho to urea), 6.90 (d, J = 9.0 Hz, |
| | solid. | 2H, aryl-dioxy ring), 6.86 (d, J = 9.4 Hz, 2H, aryl-dioxy ring), 6.70 (d, J = 9.3 |
| | Rₜ: 2.88 | Hz, 2H, aryl C—H ortho to phenol), 4.18-4.27 (m, 1H, CH(OH)), 4.05 (t, J = 4.6 |
| | (S 1a), | Hz, 2H, CH₂OArO), 4.00 (dd, J = 9.8/4.9, 1H, ArOCH₂CH(OH)), 3.95 (dd, J = |
| | 9.84 (S | 9.8/5.3, 1H, ArOCH₂CH(OH)), 3.79 (t, J = 4.7 Hz, 2H, OCH₂CH₂OArO), 3.51 (t, |
| | 3) | J = 5.3 Hz, 2H, CH₂CH₂NH), 3.37 (d, J = 6.9 Hz, 2H, ^{c}PrCH₂O), 3.28-3.35, |
| | | 3.15-3.24 (m, 4H, CH(OH)CH₂NH, NHCH₂CH₂), 1.01-1.13 (m, 1H, ^{c}Pr CH), |
| | | 0.50-0.57 (m, 2H, ^{c}Pr CH₂)*, 0.19-0.26 (^{c}Pr CH₂)*. *Refers to cis-protons |
| | | of ^{c}Pr ring. |
| 46b | 113-115 | (DMSO-d₆): δ 8.92 (br s, 1H, phenol), 8.22 (s, 1H, NH(C═O)NHAr), 7.28 (dd, J = |
| | Rₜ: 3.47 | 8.3/5.8 Hz, 2H, 3-H and 5-H of fluorophenyl ring), 7.14 (d, J = 8.8 Hz, 2H, |
| | (S 1b), | aryl C—H ortho to urea), 7.08 (dd, J = 8.9/8.9 Hz, 2H, 2-H and 6-H of |
| | 11.50 (S | flurophenyl ring), 6.82, 6.85 (d, J = 9.3 Hz, 2 x 2H, aryl-dioxy ring), 6.62 (d, J = |
| | 3). | 8.8 Hz, 2H, aryl C—H ortho to phenol), 6.03 (t, J = 5.2 Hz, 1H, NH(C═O)NHAr), |
| | | 5.01 (br s, 1H, NH), 3.99 (t, J = 4.3 Hz, 2H, CH₂OAr), 3.78-3.93 (m, 3H, |
| | | CH(OH), ArOCH₂), 3.69 (t, J = 4.3 Hz, 2H, CH₂CH₂OAr), 3.64 (t, J = 6.9 Hz, |
| | | 2H, FC₆H₄CH₂CH₂O), 3.11-3.19 (m, 2H, NHCH₂CH₂), 2.81 (t, J = 6.8 Hz, 2H, |
| | | FC₆H₄CH₂), 2.58-2.76 (m, 4H, CH(OH)CH₂NH, NHCH₂CH₂). |
| 46d | 96-98 | (MeOD-d₄): δ 7.12 (d, J = 8.9 Hz, 2H, aryl C—H ortho to urea), 6.87 (d, J = 9.0 |
| | Rₜ: 2.29 | Hz, 2H, aryl-dioxy ring), 6.84 (d, J = 9.0 Hz, 2H, aryl-dioxy ring), 6.70 (d, J = |
| | (S 1b), | 8.9 Hz, 2H, aryl C—H ortho to phenol), 4.01-4.08 (m, 1H, CH(OH)), 4.03 (t, J = |
| | 8.72 (S | 4.6 Hz, 2H, CH₂OArO), 3.87-3.95 (m, 2H, ArOCH₂CH(OH)), 3.75 (t, J = 4.7 |
| | 3). | Hz, 2H, OCH₂CH₂OArO), 3.59 (q, J = 7.0 Hz, 2H, CH₃CH₂), 3.34 (t, J = 6.1 Hz, |
| | | 2H, CH₂CH₂NH), 2.74-2.90 (m, 4H, CH(OH)CH₂NH, NHCH₂CH₂), 1.21 (t, J = |
| | | 7.0 Hz, 3H, CH₃). |
| 46e | Rₜ: 3.95 | (DMSO-d₆): δ 8.95 (s, 1H, NH(C═O)NHAr), 8.26 (br s, 1H, formate HCO₂⁻), |
| | (S 1b), | 7.67 (dd, J = 2.0/2.0 Hz, 1H, aryl 2-H), 7.22 (dd, J = 7.7/7.7 Hz, 1H, aryl 5-H), |
| | 11.73 (S | 7.18 (ddd, J = 8.3/1.8/1.8 Hz, 1H, aryl 6-H), 6.91 (ddd, J = 7.4/1.8/1.8 Hz, 1H, |
| | 3). | aryl 4-H), 6.80-6.88 (m, 4H, aryl-dioxy C—H), 6.43 (t, J = 5.1 Hz, 1H, |
| | | NH(C═O)NHAr), 3.99 (t, J = 4.7 Hz, 2H, CH₂OAr), 3.78-3.92 (m, 3H, |
| | | CH(OH), ArOCH₂), 3.66 (t, J = 4.7 Hz, 2H, CH₂CH₂OAr), 3.28 (d, J = 7.1 Hz, |
| | | 2H, ^{c}PrCH₂O), 3.18 (dt, J = 5.9/5.6 Hz, 2H, NHCH₂CH₂), 2.72 (dd, J = 11.9/4.0 |
| | | Hz, 1H, CH(OH)CH₂NH), 2.66 (t, J = 6.2 Hz, 2H, NHCH₂CH₂), 2.61 (dd, J = |
| | | 12.1/6.8 Hz, 1H, CH(OH)CH₂NH), 1.06-0.94 (m, 1H, ^{c}Pr CH), 0.42-0.49 (m, |
| | | 2H, ^{c}Pr CH₂)*, 0.13-0.19 (^{c}Pr CH₂)*. *Refers to cis-protons of ^{c}Pr ring. |
| 46f | Rₜ: 3.93 | (DMSO-d₆): δ 9.02 (s, 1H, NH(C═O)NHAr), 8.27 (br s, 1H, formate HCO₂⁻), |
| | (S 1b), | 7.67 (dd, J = 2.0/2.0 Hz, 1H, aryl 2-H), 7.22 (dd, J = 7.7/7.7 Hz, 1H, aryl 5-H), |
| | 11.69 (S | 7.18 (ddd, J = 8.3/1.8/1.8 Hz, 1H, aryl 6-H), 6.91 (ddd, J = 7.4/1.8/1.8 Hz, 1H, |
| | 3). | aryl 4-H), 6.86, 6.83 (d, J = 9.2 Hz, 2 x 2H, aryl-dioxy C—H), 6.51 (t, J = 5.3 Hz, |
| | | 1H, NH(C═O)NHAr), 3.99 (t, J = 4.7 Hz, 2H, CH₂OAr), 3.78-3.92 (m, 3H, |
| | | CH(OH), ArOCH₂), 3.66 (t, J = 4.7 Hz, 2H, CH₂CH₂OAr), 3.48 (q, J = 7.0 Hz, |
| | | 2H, CH₃CH₂), 3.19 (dt, J = 6.2/5.8 Hz, 2H, NHCH₂CH₂), 2.74 (dd, J = 12.1/4.3 |
| | | Hz, 1H, CH(OH)CH₂NH), 2.68 (t, J = 6.2 Hz, 2H, NHCH₂CH₂), 2.63 (dd, J = |
| | | 12.1/6.6 Hz, 1H, CH(OH)CH₂NH), 1.20 (t, J = 6.6 Hz, 3H, CH₃). |
| 46g | Rₜ: 3.17 | (DMSO-d₆): δ 7.98 (t, J = 5.5 Hz, 1H, NH(C═O)), 7.00-7.10 (m, 2H, |
| | (S 1b), | hydroxyaryl 4H, 6H), 6.85 (s, 4H, aryl-dioxy ring), 6.77 (dd, J = 7.9/0.9 Hz, 1H, |
| | 9.28 (S | aryl 3H), 6.71 (ddd, J = 7.4/7.4/1.1 Hz, 1H, aryl 5H), 3.99 (t, J = 4.6 Hz, 2H, |
| | 3) | CH₂CH₂OAr), 3.72-3.92 (m, 3H, CH(OH), ArOCH₂), 3.65 (t, J = 4.7 Hz, 2H, |
| | | OCH₂CH₂OAr), 3.48 (q, J = 7.0 Hz, 2H, CH₃CH₂), 3.38 (s, 2H, NH(C═O)CH₂), |
| | | 3.16 (dt, J = 6.3/5.9 Hz, 2H, NHCH₂CH₂NH), 2.54-2.74 (m, 4H, |
| | | CH(OH)CH₂NH, NHCH₂CH₂NH), 1.11 (t, J = 7.0 Hz, 3H, CH₃) |
| 46h | Rₜ: 2.95 | (DMSO-d₆): δ 9.28 (s, 1H, phenol OH), 7.99 (t, J = 5.16 Hz, 1H, NH(C═O)), |
| | (S 1b), | 7.04 (dd, J = 7.8.7.8 Hz, 1H, aryl 5H), 6.85 (s, 4H, aryl-dioxy ring), 6.56-6.70 |
| | 8.59 (S 3 | (m, 3H, aryl 2H, 4H, 6H), 5.00 (s, 1H, CH(OH)), 4.00 (t, J = 4.6 Hz, 2H, |
| | | OCH₂CH₂OAr), 3.74-3.90 (m, 3H, CH(OH), ArOCH₂), 3.65 (t, J = 4.6 Hz, 2H, |
| | | OCH₂CH₂OAr), 3.48 (q, J = 6.9 Hz, 2H, CH₃CH₂), 3.30 (s, 2H, CH₂Ar), 3.11- |
| | | 3.17 (m, 2H, NHCH₂CH₂NH), 2.56-2.80 (m, 4H, CH(OH)CH₂NH, |
| | | NHCH₂CH₂NH), 1.12 (t, J = 7.0 Hz, 3H, CH₃). |
| 46i | Rₜ: 3.34 | (DMSO-d₆): δ 9.19 (s, 1H, phenol OH), 7.90 (t, J = 5.5 Hz, 1H, NH(C═O)), |
| | (S 1b), | 7.02 (d, J = 8.5 Hz, 2H, aryl 2H, 6H), 6.85 (s, 4H, aryl-dioxy ring), 6.66 (d, J = |
| | 9.53 (S | 8.5 Hz, 2H, aryl 3H, 5H), 5.00 (s, 1H, CH(OH)), 4.00 (t, J = 4.6 Hz, 2H, |
| | 3) | OCH₂CH₂OAr), 3.76-3.92 (m, 3H, ArOCH₂ CH(OH)), 3.68 (t, J = 3.4 Hz, 2H, |
| | | OCH₂CH₂OAr), 3.28 (d, J = 6.7 Hz, 2H, ^{c}PrCH₂O), 3.25 (s, 2H, NH(C═O)CH₂), |
| | | 3.13 (dt, J = 5.8/5.8 Hz, 2H, NHCH₂CH₂NH), 2.55-2.75 (m, 4H, |
| | | CH(OH)CH₂NH, NHCH₂CH₂NH), 0.92-1.06 (m, 1H, ^{c}Pr CH), 0.40-0.50 (m, |
| | | 2H, ^{c}Pr CH₂)*, 0.10-0.22 (m, 2H, ^{c}Pr CH₂)*. *Refers to cis-protons of ^{c}Pr ring. |
| 46k | Rₜ: 2.19 (s 4), 2.34 (s 5); 1H nmr data not included - similar to 46l | |
| 46l | Rₜ: 1.95 | (DMSO-d₆): δ 8.99 (s, 1H, phenol OH), 8.51 (1H, NH(C═O)NHAr), 7.15 (t, J = |
| | (s 4), | 8.6 Hz, 2H, aryl C—H ortho to urea), 6.86 (s, 4H, aryl-dioxy ring), 6.63 (t, J = 8.6 |
| | 2.13 (s | Hz, 2H, aryl C—H ortho to phenol), 6.37 (t, J = 5.7 Hz, 1H, NH(C═O)NHAr), |
| | 5) | 5.80 (br, 1H, NH), 4.18 (br, d, J = 3.9 Hz, 1H, OCH₂CH(OH)), 3.85-3.99 (m, |
| | | 5H, CH₂OAr, CH(OH), ArOCH₂), 3.65 (t, J = 4.7 Hz, 2H, iPrOCH₂), 3.38 (q, |
| | | 2H, J = 5.8 Hz, NHCH₂CH₂), 2.94-3.23 (m, 5H, CH(OH)CH₂NH, NHCH₂CH₂ |
| | | (CH₃)₂CH), 1.10 (d, 6H, (CH₃)₂CH). |

| | |
|---|---|
| 46m | 127-130; Rₜ: 2.23 (s 4), 2.44 (s 5) |
| 46n | Rₜ: 1.99 (s 4), 2.15 (s 5) |
| 46o | 107-111; Rₜ: 2.18 (s 4), 2.34 (s 5) |
| 46p | Rₜ: 1.91 (s 4), 2.09 (s 5); 1H nmr data not included - similar to 46l |

### General Procedure for Synthesis of Individual Enantiomers of 1-(2-(3-(4-(2-(cyclopentyloxy)ethoxy)phenoxy)-2-hydroxypropylamino)ethyl)-3-(3-chlorophenyl)urea (471)

The appropriate chiral epoxide was synthesised by alkylation of 40c with either (R)- or (S)-glycidyl nosilate according to the procedure reported by Sharpless and Al. JOC, 54(6), 1989, 1295-1304. Subsequent confirmation of chiral purity was assessed using Mosher ester Fluorine NMR also described therein. Subsequently, the epoxides (1 eq) were opened using 16k (1.3 eq) in HFIP (4 mL) at 70° C. for 24 hours, with reaction monitoring using LC-MS. Purification was achieved using FCC (eluent 1M NH₃ in MeOH/DCM, gradient method).

Epoxide ee: 98% for both (R)- and (S)-epoxides based on Mosher ester analysis

### (R)-1-(2-(3-(4-(2-(Cyclopentyloxy)ethoxy)phenoxy)-2-hydroxypropylamino)ethyl)-3-(3-chlorophenyl)urea ((R)-471)

White, solid. Yield=113 mg (46%, based on (R)-epoxide 0.5 mmol), purity >95%.

Measurement of Alpha D: temperature=23° C.; concentration=10.35 mg/ml; Alpha=+0.12/+0.11; AlphaD calc=+5.3

### (S)-1-(2-(3-(4-(2-(Cyclopentyloxy)ethoxy)phenoxy)-2-hydroxypropylamino)ethyl)-3-(3-chlorophenyl)urea ((S)-471)

White solid. Yield=132 mg (40%, based on (S)-epoxide 0.67 mmol), purity >95%

Measurement of Alpha D: temperature=23° C.; concentration=12.0 mg/ml; Alpha=−0.131-0.13; AlphaD calc=−5.4.

### 1-(2-(3-(4-(2-(Cyclopentyloxy)ethoxy)phenoxy)-2-hydroxypropylamino)ethyl)-3-(2-hydroxyphenyl)urea (47t)

To 41c (51 mg, 0.18 mmol) was added TEA (75 μl, 0.53 mmol, 3 eq), 30a (83 mg, 0.36 mmol, 2 eq) and isopropyl alcohol (5 mL). The mixture was stirred under reflux for 16 hours. After removal of all volatiles under reduced pressure, the crude residue was purified by PLC (eluent NH₃/MeOH/DCM 2:5:93) to give 18 mg of beige semi-solid.

### 1-(2-(3-(4-(2-(Cyclopentyloxy)ethoxy)phenoxy)-2-hydroxypropylamino)ethyl)-3-(3-hydroxyphenyl)urea (47u)

Epoxide opening of 41c with 30b, was carried out as described for 47t. Purification was achieved via PLC (eluent NH₃/MeOH/DCM 2:10:88) to give 21 mg of beige semi-solid.

### 1-(2-(3-(4-(2-(Cyclopentyloxy)ethoxy)phenoxy)-2-hydroxypropylamino)ethyl)-3-(4-nitrophenyl)urea (47w)

41c (100 mg, 0.36 mmol), 16s (112 mg, 0.43 mmol, 1.2 eq) and TEA (0.060 mL, 0.43 mmol, 1.2 eq) were dispersed in propan-2-ol/acetonitrile/water (7:2:1, 3 mL) and heated at 90° C. in the MW reactor on a dynamic program (maximum pressure 250 psi, maximum power 300W) for 60 minutes. After removal of volatiles under reduced pressure, the crude product was purified by FCC (eluent 1M NH₃ in MeOH/DCM 0:100 to 15:85), to give 74 mg of pale yellow solid (41%).

### 1-(2-(3-(4-(2-(Cyclopentyloxy)ethoxy)phenoxy)-2-hydroxypropylamino)ethyl)-3-(4-(2-fluoroethoxy)phenyl)urea (47×)

41c and 38d were reacted together according to the procedure described for the synthesis of 47w to give 43 mg (23%) of a white solid.

### Methyl 3-(3-(2-(3-(4-(2-(cyclopentyloxy)ethoxy)phenoxy)-2-hydroxypropylamino)ethyl)ureido)benzoate (47y)

41c and 16t were reacted together according to the procedure described for the synthesis of 47w to give 44 mg (24%) of a white solid.

### Methyl 4-(3-(2-(3-(4-(2-(cyclopentyloxy)ethoxy)phenoxy)-2-hydroxypropylamino)ethyl)ureido)benzoate (47z)

41c and 16u were reacted together according to the procedure described for the synthesis of 47w to give 68 mg (37%) of a white solid.

### 3-(3-(2-(3-(4-(2-(Cyclopentyloxy)ethoxy)phenoxy)-2-hydroxypropylamino)ethyl)ureido)benzoic acid hydrochloride (47aa)

47y (38 mg) was dissolved in aq. 2M HCl (10 mL) and heated under reflux overnight. The mixture was concentrated before freeze-drying to give 32 mg of a pale yellow solid (80%).

### 4-(3-(2-(3-(4-(2-(Cyclopentyloxy)ethoxy)phenoxy)-2-hydroxypropylamino)ethyl)ureido)benzoic acid hydrochloride (47bb)

47z (41 mg) was dissolved in aq. 2M HCl (10 mL) and heated under reflux overnight. The mixture was concentrated before freeze-drying to give 32 mg of a pale yellow solid (74%).

### 1-(2-Phenoxyethylamino)-3-(4-(2-(cyclopentyloxy)ethoxy)phenoxy)propan-2-ol (48)

41c (50 mg, 0.18 mmol) and 2-phenoxyethylamine (47 μL, 0.36 mmol, 2 eq) were dissolved in propan-2-ol (3 mL) before heating under reflux overnight. After removal of all solvent under vacuum, the crude residue was purified via PLC (eluent NH₃/MeOH/DCM 2:5:93) to give 51 mg of white solid.

### N-(2-(3-(4-(2-(Cyclopentyloxy)ethoxy)phenoxy)-2-hydroxypropylamino)ethyl)-2-phenylacetamide Hydroformate (49)

41c (55 mg, 0.20 mmol) was opened with 18 according to the method described for 47t. Purification via PLC (eluent NH₃/MeOH/DCM 2:8:90) and preparative HPLC afforded 15 mg of white solid.

### 4-(3-(4-(2-(Cyclopentyloxy)ethoxy)phenoxy)-2-hydroxypropylamino)-N-phenylbutanamide (50)

41c (50 mg, 0.18 mmol) was opened with 26 according to the method described for 47t. Purification via PLC (eluent NH₃/MeOH/DCM 2:8:90) and preparative HPLC afforded 19 mg of white solid.

### 2-(3-(4-(2-(Cyclopentyloxy)ethoxy)phenoxy)-2-hydroxypropylamino)ethyl phenylcarbamate (51)

41c (50 mg, 0.18 mmol) was opened with 34 according to the method described for 47t. Purification via PLC (eluent NH₃/MeOH/DCM 1:5:94) afforded 66 mg of white solid.

### 1-(2-(3-(4-(2-(Cyclopentyloxy)ethoxy)phenoxy)-2-hydroxypropylamino)ethyl)-3-phenylthiourea (52)

41c (50 mg, 0.18 mmol) was opened with 20 according to the method described for 47t. Purification via PLC (eluent NH₃/MeOH/DCM 1:5:94) afforded 31 mg of white solid.

### N-(2-(3-(4-(2-(Cyclopentyloxy)ethoxy)phenoxy)-2-hydroxypropylamino)ethyl)-benzylsulfonamide (53)

41c (50 mg, 0.18 mmol) was opened with 22 according to the method described for 47t. Purification via PLC (eluent NH₃/MeOH/DCM 1:5:94) afforded 79 mg of white solid.

### 1-(2-(3-(4-(2-(Cyclopentyloxy)ethoxy)phenoxy)-2-hydroxypropylamino)ethyl)-3-(3-fluoro-4-hydroxyphenyl)urea Hydroformate (54)

41c (50 mg, 0.18 mmol), TEA (50 μL, 0.36 mmol, 2 eq) and 38 (67 mg, 0.27 mmol, 1.5 eq) were dissolved in EtOH (2 mL) before exposing to MW conditions (140° C., 80W, 250 psi) for 8 minutes. Purification via PLC (eluent NH₃/MeOH/DCM 1:5:94) afforded 13 mg of white solid.

### 1-(2-(3-(4-(2-(Cyclopentyloxy)ethoxy)phenoxy)-2-hydroxypropylamino)ethyl)-3-(3,4-difluorophenyl)urea (54a)

Epoxide opening of 41c with 22a, was carried out as described for 47t. Purification was achieved via PLC (eluent NH₃/MeOH/DCM 2:10:88) to give 50 mg of yellow solid.

### 1-(3-Chloro-4-methoxyphenyl)-3-(2-(3-(4-(2-(cyclopentyloxy)ethoxy)phenoxy)-2-hydroxypropylamino)ethyl)urea (54b)

22i (mixture of 4-methoxy and 4-hydroxy compounds) and 41c were reacted as described in the general method for epoxide openings in HFIP. The title compound was isolated during FCC purification and underwent recrystallisation from tert-butyl methyl ether and PE.

### 1-(3-Chloro-4-hydroxyphenyl)-3-(2-(3-(4-(2-(cyclopentyloxy)ethoxy)phenoxy)-2-hydroxypropylamino)ethyl)urea (54c)

The title compound was isolated from the FCC purification of 54b, and recrystallised from tert-butyl methyl ether and PE.

Table 17 lists the ¹H NMR spectral data for selected compounds from Figure 4:

| | m.p; | |
|---|---|---|
| Cpd | HPLC | ¹H NMR |
| 47a | 109-115 | (DMSO-d₆): δ 8.61 (s, 1H, NH(C═O)NHAr), 7.38 (dd, J = 8.7/1.1 Hz, 2H, 2-H |
| | Rₜ: 3.62 (S | and 6-H phenyl ring), 7.20 (dd, J = 7.4/7.4 Hz, 2H, 3-H and 5-H phenyl ring), |
| | 1b), 12.67 | 6.81-6.89 (m, 5H, 4-H phenyl ring, aryl-dioxy ring), 6.22 (t, J = 5.4 Hz, 1H, |
| | (S 3). | NH(C═O)NHAr), 5.07 (br s, 1H, NH) 3.79-3.97 (m, 6H, CH₂OAr, ^{c}Pe CH, |
| | | CH(OH), ArOCH₂), 3.61 (t, J = 4.8 Hz, 2H, ^{c}PeOCH₂), 3.19 (dt, J = 5.8/5.8 Hz, |
| | | 2H, NHCH₂CH₂), 2.75 (dd, J = 12.0/4.0 Hz, 1H, CH(OH)CH₂NH), 2.68 (t, J = 6.0 |
| | | Hz, 2H, NHCH₂CH₂), 2.63 (dd, J = 12.2/6.8 Hz, 1H, CH(OH)CH₂NH), 1.52- |
| | | 1.74 (m, 6H, ^{c}Pe CH₂), 1.40-1.52 (m, 2H, ^{c}Pe CH₂). |
| 47b | 114-115 | (DMSO-d₆): δ 7.80 (d, J = 7.6 Hz, 1H, aryl 6-H), 7.70 (s, 1H, NH(C═O)NHAr), |
| | Rₜ: 4.07 (S | 7.05-7.11 (m, 2H, C—H tolyl ring), 6.80-6.87 (m, 5H, C—H aryl-dioxy ring, C—H |
| | 1b), 7.60 | tolyl ring), 6.60 (t, J = 5.4 Hz, 1H, NH(C═O)NHAr), 4.95 (br s, 1H, NH), 3.77- |
| | (S 3). | 4.01 (m, 6H, CH₂OAr, ^{c}Pe CH, CH(OH), ArOCH₂), 3.61 (t, J = 4.9 Hz, 2H, |
| | | ^{c}PeOCH₂), 3.17 (dt, J = 5.9/5.9 Hz, 2H,), 2.70 (dd, J = 11.8/4.2 Hz, 1H, |
| | | CH(OH)CH₂NH), 2.64 (t, J = 6.0 Hz, 3H, NHCH₂CH₂), 2.59 (dd, J = 11.7/6.3 |
| | | Hz, CH(OH)CH₂NH), 2.17 (s, 3H, CH₃), 1.52-1.76 (m, 6H, ^{c}Pe CH₂), 1.40- |
| | | 1.52 (m, 2H, ^{c}Pe CH₂). |
| 47c | Rₜ: 4.25 (S | 112-114 (DMSO-d₆): δ 8.51 (s, 1H, NH(C═O)NHAr), 7.21 (s, 1H, aryl 2-H), |
| | 1b), 8.28 | 7.10 (d, J = 8.4 Hz, 1H, aryl 6-H), 7.08 (dd, J = 7.6/7.6 Hz, 1H, aryl 5-H), 6.85, |
| | (S 3). | 6.83 (d, J = 9.2 Hz, 2 x 2H, C—H aryl-dioxy ring), 6.46 (d, J = 7.4 Hz, 1H aryl 4- |
| | | H), 6.20 (t, J = 5.4 Hz, 1H, NH(C═O)NHAr), 5.09 (br s, 1H, NH), 3.78-4.00 (m, |
| | | 6H, CH₂OAr, ^{c}Pe CH, CH(OH), ArOCH₂), 3.61 (t, J = 4.9 Hz, 2H, ^{c}PeOCH₂), |
| | | 3.19 (dt, J = 5.9/5.9 Hz, 2H, NHCH₂CH₂), 2.77 (dd, J = 11.8/3.6 Hz, 1H, |
| | | CH(OH)CH₂NH), 2.61-2.73 (m, 3H, CH(OH)CH₂NH, NHCH₂CH₂), 2.23 (s, |
| | | 3H, CH₃), 1.52-1.75 (m, 6H, ^{c}Pe CH₂), 1.41-1.52 (m, 2H, ^{c}Pe CH₂). |
| 47d | 142-144 | (DMSO-d₆): δ 8.46 (s, 1H, NH(C═O)NHAr), 7.26 (d, J = 8.4 Hz, 2H, aryl 2-H |
| | Rₜ: 4.18 (S | and 6-H), 7.01 (d, J = 8.3 Hz, 2H, aryl 3-H and 5-H), 6.82, 6.85 (d, J = 9.2 Hz, 2 |
| | 1b), 13.52 | x 2H, C—H aryl-dioxy ring), 6.14 (t, J = 5.5 Hz, 1H, NH(C═O)NHAr), 5.04 (br s, |
| | (S 3). | 1H, NH), 3.79-3.97 (m, 6H, CH₂OAr, ^{c}Pe CH, CH(OH), ArOCH₂), 3.61 (t, J = |
| | | 4.9 Hz, 2H, ^{c}PeOCH₂), 3.18 (dt, J = 5.8/5.8 Hz, 2H, NHCH₂CH₂), 2.74 (dd, J = |
| | | 11.9/3.6 Hz, 1H, CH(OH)CH₂NH), 2.59-2.70 (m, 3H, CH(OH)CH₂NH, |
| | | NHCH₂CH₂), 2.21 (s, 3H, CH₃), 152-1.76 (m, 6H, ^{c}Pe CH₂), 1.41-1.52 (m, |
| | | 2H, ^{c}Pe CH₂). |
| 47e | 78-83 | (DMSO-d₆): δ 8.07 (dd, J = 7.6/2.0 Hz, 1H, aryl 6-H), 7.97 (s, 1H, |
| | Rₜ: 3.77 (S | NH(C═O)NHAr), 6.92-6.99 (m, 2H, C—H methoxyphenyl ring, NH(C═O)NHAr), |
| | 1b), 12.87 | 6.79-6.89 (m, 6H, C—H aryl-dioxy ring, C—H methoxyphenyl ring), 5.07 (br s, |
| | (S 3). | 1H, NH), 3.76-4.00 (m, 6H, CH₂OAr, ^{c}Pe CH, CH(OH), ArOCH₂), 3.82 (s, 3H, |
| | | CH₃), 3.61 (t, J = 4.8 Hz, 2H, ^{c}PeOCH₂), 3.18 (dt, J = 5.9/5.9 Hz, 2H, |
| | | NHCH₂CH₂), 2.74 (dd, J = 11.9/3.9 Hz, 1H, CH(OH)CH₂NH), 2.59-2.70 (m, |
| | | 3H, CH(OH)CH₂NH, NHCH₂CH₂), 1.52-1.75 (m, 6H, ^{c}Pe CH₂), 1.40-1.51 |
| | | (m, 2H, ^{c}Pe CH₂). |
| 47f | 103-110 | (DMSO-d₆): δ 8.63 (s, 1H, NH(C═O)NHAr), 7.14 (dd, J = 2.2/2.2 Hz, 1H, aryl 2- |
| | Rₜ: 3.40 (S | H), 7.10 (dd, J = 8.1/8.1 Hz, 1H, aryl 5-H), 6.79-6.89 (m, 5H, C—H aryl-dioxy |
| | 1b), 12.42 | ring, aryl 6-H), 6.46 (ddd, J = 8.2/2.4/0.6 Hz, 1H aryl 4-H), 6.22 (t, J = 5.4 Hz, |
| | (S 3). | 1H, NH(C═O)NHAr), 5.05 (br s, 1H, NH), 3.79-3.97 (m, 6H, CH₂OAr, ^{c}Pe CH, |
| | | CH(OH), ArOCH₂), 3.69 (s, 3H, CH₃), 3.61 (t, J = 4.8 Hz, 2H, ^{c}PeOCH₂), 3.18 |
| | | (dt, J = 5.9/5.9 Hz, 2H, NHCH₂CH₂), 2.73 (dd, J = 11.9/3.9 Hz, 1H, |
| | | CH(OH)CH₂NH), 2.50-2.70 (m, 3H, CH(OH)CH₂NH, NHCH₂CH₂), 1.52-1.74 |
| | | (m, 6H, ^{c}Pe CH₂), 1.41-1.52 (m, 2H, ^{c}Pe CH₂). |
| 47g | 121-125 | (DMSO-d₆): δ 8.37 (s, 1H, NH(C═O)NHAr), 7.10 (d, J = 9.0 Hz, 2H, aryl 2-H |
| | Rₜ: 3.37 (S | and 6-H), 6.82-6.87 (m, 4H, C—H aryl-dioxy ring), 6.80 (d, J = 9.0 Hz, 2H, aryl |
| | 1b), 12.02 | 3-H and 5-H), 6.07 (t, J = 5.5 Hz, 1H, NH(C═O)NHAr), 5.00 (br s, 1H, NH), 3.75- |
| | (S 3). | 4.00 (m, 6H, CH₂OAr, ^{c}Pe CH, CH(OH), ArOCH₂), 3.68 (s, 3H, CH₃), 3.61 (t, |
| | | J = 4.7 Hz, 2H, ^{c}PeOCH₂), 3.15 (dt, J = 5.8/5.8 Hz, 2H, NHCH₂CH₂), 2.55- |
| | | 2.76 (m, 4H, CH(OH)CH₂NH, NHCH₂CH₂), 1.52-1.74 (m, 6H, ^{c}Pe CH₂), |
| | | 1.41-1.52 (m, 2H, ^{c}Pe CH₂). |
| 47h | 115-117 | (DMSO-d₆): δ 8.38 (s, 1H, NH(C═O)NHAr), 8.12 (dd, J = 8.3/1.5 Hz, 1H, aryl 6- |
| | Rₜ: 4.05 (S | H), 7.16 (ddd, J = 11.8/8.2/1.4 Hz, 1H, aryl 3-H), 7.06 (dd, J = 8.2/8.2 Hz, 1H, |
| | 1b), 12.35 | aryl 5-H), 6.87-6.94 (m, 1H, aryl 4-H), 6.85, 6.82 (d, J = 9.3 Hz, 2 x 2H, C—H |
| | (S 3). | aryl-dioxy ring), 6.70 (t, J = 5.4 Hz, 1H, NH(C═O)NHAr) 4.99 (br s, 1H, NH), |
| | | 3.77-4.01 (m, 6H, CH₂OAr, ^{c}Pe CH, CH(OH), ArOCH₂), 3.61 (t, J = 4.8 Hz, |
| | | 2H, ^{c}PeOCH₂), 3.18 (dt, J = 6.0/6.0 Hz, 2H, NHCH₂CH₂), 2.71 (dd, J = 11.8/3.9 |
| | | Hz, 1H, CH(OH)CH₂NH), 2.56-2.67 (m, 3H, CH(OH)CH₂NH, NHCH₂CH₂), |
| | | 1.51-1.75 (m, 6H, ^{c}Pe CH₂), 1.41-1.51 (m, 2H, ^{c}Pe CH₂). |
| 47i | 108-110 | (DMSO-d₆): δ 8.86 (s, 1H, NH(C═O)NHAr), 7.45 (ddd, J = 12.3/2.2/2.2 Hz, 1H, |
| | Rₜ: 4.02 (S | aryl 2-H), 7.22 (ddt, J = 8.2/8.2.2/8.2 Hz, 1H, aryl 5-H), 7.00 (ddd, J = 8.2/1.2 |
| | 1b), 13.32 | Hz, 1H, aryl 6-H), 6.79-6.89 (m, 4H, C—H aryl-dioxy ring), 6.68 (ddd, J = |
| | (S 3). | 8.2/8.2/2.1 Hz, 1H, aryl 4-H), 6.28 (t, J = 5.4 Hz, 1H, NH(C═O)NHAr), 5.04 (br |
| | | s, 1H, NH), 3.77-4.01 (m, 6H, CH₂OAr, ^{c}Pe CH, CH(OH), ArOCH₂), 3.61 (t, J = |
| | | 4.8 Hz, 2H, ^{c}PeOCH₂), 3.18 (dt, J = 5.8/5.8 Hz, 2H, NHCH₂CH₂), 2.57-2.79 |
| | | (m, 4H, CH(OH)CH₂NH, NHCH₂CH₂), 1.52-1.75 (m, 6H, ^{c}Pe CH₂), 1.40- |
| | | 1.52 (m, 2H, ^{c}Pe CH₂). |
| 47j | 130-133 | (DMSO-d₆): δ 8.69 (s, 1H, NH(C═O)NHAr), 7.39 (dd, J = 9.1/5.0 Hz, 2H, 2-H |
| | Rₜ: 4.12 (S | and 6-H fluorophenyl ring), 7.04 (dd, J = 8.9/8.9 Hz, 2H, 3-H and 5-H |
| | 1b), 13.20 | fluorophenyl ring), 6.80-6.88 (m, 4H, aryl-dioxy ring), 6.23 (t, J = 5.4 Hz, 1H, |
| | (S 3). | NH(C═O)NHAr), 5.13 (br s, 1H, NH), 3.78-4.00 (m, 6H, CH₂OAr, ^{c}Pe CH, |
| | | CH(OH), ArOCH₂), 3.61 (t, J = 4.8 Hz, 2H, ^{c}PeOCH₂), 3.15-3.24 (m, 2H, |
| | | NHCH₂CH₂), 2.78 (dd, J = 11.9/3.7 Hz, 1H, CH(OH)CH₂NH), 2.61-2.74 (m, |
| | | 3H, CH(OH)CH₂NH, NHCH₂CH₂), 1.52-1.75 (m, 6H, ^{c}Pe CH₂), 1.40-1.52 |
| | | (m, 2H, ^{c}Pe CH₂). |
| 47k | 99-103 | (DMSO-d₆): δ 8.14 (dd, J = 8.3/1.3 Hz, 1H, aryl 6-H), 8.09 (s, 1H, |
| | Rₜ: 3.50 (S | NH(C═O)NHAr), 7.38 (dd, J = 8.0/1.4 Hz, 1H, aryl 3-H), 7.22 (ddd, J = |
| | 1b), 13.09 | 7.8/7.8/1.4 Hz, 1H, aryl 5-H), 7.07 (t, J = 5.2 Hz, 1H, NH(C═O)NHAr), 6.93 |
| | (S 3). | (ddd, J = 7.8/7.8/1.5 Hz, 1H, aryl 4-H), 6.79-6.86 (m, 4H, C—H aryl-dioxy ring), |
| | | 4.98 (br s, 1H, NH), 3.73-4.01 (m, 6H, CH₂OAr, ^{c}Pe CH, CH(OH), ArOCH₂), |
| | | 3.61 (t, J = 4.8 Hz, 2H, ^{c}PeOCH₂), 3.18 (dt, J = 5.8/5.8 Hz, 2H, NHCH₂CH₂), |
| | | 2.57-2.72 (m, 4H, CH(OH)CH₂NH, NHCH₂CH₂), 1.51-1.74 (m, 6H, ^{c}Pe |
| | | CH₂), 1.41-1.51 (m, 2H, ^{c}Pe CH₂). |
| 47l | 102-109 | (DMSO-d₆): δ 8.85 (s, 1H, NH(C═O)NHAr), 7.67 (dd, J = 2.0/2.0 Hz, 1H, aryl 2- |
| | Rₜ: 3.88 (S | H), 7.22 (dd, J = 8.1/8.1 Hz, 1H, aryl 5-H), 7.16 (ddd, J = 8.2/1.8/1.2 Hz, 1H, |
| | 1b), 13.87 | aryl 6-H), 6.92 (ddd, J = 7.7/2.1/1.2 Hz, 1H, aryl 4-H), 6.85 (d, J = 9.3 Hz, 2H, |
| | (S 3). | C—H aryl-dioxy ring), 6.82 (d, J = 9.3 Hz, 2H, C—H aryl-dioxy ring), 6.29 (t, J = |
| | | 5.4 Hz, 1H, NH(C═O)NHAr), 5.03 (br s, 1H, NH), 3.77-4.00 (m, 6H, CH₂OAr, |
| | | ^{c}Pe CH, CH(OH), ArOCH₂), 3.61 (t, J = 4.8 Hz, 2H, ^{c}PeOCH₂), 3.18 (dt, J = |
| | | 5.9/5.9 Hz, 2H, NHCH₂CH₂), 2.57-2.77 (m, 4H, CH(OH)CH₂NH, NHCH₂CH₂), |
| | | 1.51-1.74 (m, 6H, ^{c}Pe CH₂), 1.40-1.51 (m, 2H, ^{c}Pe CH₂). |
| 47m | 133-140 | (DMSO-d₆): δ 8.76 (s, 1H, NH(C═O)NHAr), 7.41, 7.25 (d, J = 8.9 Hz, 2 x 2H, C—H |
| | Rₜ: 3.80 (S | of chlorophenyl ring), 6.81-6.86 (m, 4H, C—H aryl-dioxy ring), 7.23 (t, J = 5.4 |
| | 1b), 13.73 | Hz, 1H, NH(C═O)NHAr), 5.00 (br s, 1H, NH), |
| | (S 3). | 3.76-3.99 (m, 6H, CH₂OAr, ^{c}Pe CH, CH(OH), ArOCH₂), 3.61 (t, J = 4.8 Hz, |
| | | 2H, ^{c}PeOCH₂), 3.17 (dt, J = 5.8/5.8 Hz, 2H, NHCH₂CH₂), 2.57-2.72 (m, 4H, |
| | | CH(OH)CH₂NH, NHCH₂CH₂), 1.52-1.74 (m, 6H, ^{c}Pe CH₂), 1.40-1.51 (m, |
| | | 2H, ^{c}Pe CH₂). |
| 47n | semi-solid. | (DMSO-d₆): δ 8.04 (dd, J = 8.3/1.5 Hz, 1H, aryl 6-H), 7.98 (s, 1H, |
| | Rₜ: 4.09 (S | NH(C═O)NHAr), 7.56 (dd, J = 8.0/1.4 Hz, 1H, aryl 3-H), 7.22-7.32 (m, 2H, aryl |
| | 1b), 13.34 | 5-H, NH(C═O)NHAr), 6.79-6.94 (m, 5H, aryl 4-H, aryl-dioxy ring), 3.76-4.15 |
| | (S 3). | (m, 6H, CH₂OAr, ^{c}Pe CH, CH(OH), ArOCH₂), 3.61 (t, J = 4.8 Hz, 2H, |
| | | ^{c}PeOCH₂), 3.18-3.56 (m, 10H, H₂O, NHCH₂CH₂), 2.55-3.13 (m, 4H, |
| | | CH(OH)CH₂NH, NHCH₂CH₂), 1.52-1.75 (m, 6H, ^{c}Pe CH₂), 1.40-1.52 (m, |
| | | 2H, ^{c}Pe CH₂). |
| 47o | 120-122 | (DMSO-d₆): δ 8.83 (s, 1H, NH(C═O)NHAr), 7.81 (dd, J = 1.9/1.9 Hz, 1H, aryl 2- |
| | Rₜ: 4.47 (S | H), 7.21 (ddd, J = 8.2/1.7/1.7 Hz, 1H, aryl 6-H), 7.16 (dd, J = 7.7/7.7 Hz, 1H, |
| | 1b), 14.42 | aryl 5-H), 7.05 (ddd, J = 7.7/1.2/1.2 Hz, 1H, aryl 4-H), 6.85 (d, J = 9.3 Hz, 2H, |
| | (S 3). | C—H aryl-dioxy ring), 6.82 (d, J = 9.3 Hz, 2H, C—H aryl-dioxy ring), 6.29 (t, J = |
| | | 5.3 Hz, 1H, NH(C═O)NHAr), 5.02 (br s, 1H, NH), 3.76-4.00 (m, 6H, CH₂OAr, |
| | | ^{c}Pe CH, CH(OH), ArOCH₂), 3.61 (t, J = 4.8 Hz, 2H, ^{c}PeOCH₂), 3.13-3.21 (m, |
| | | 2H, NHCH₂CH₂), 2.71 (dd, J = 11.7/3.7 Hz, 1H, CH(OH)CH₂NH), 2.67 (dd, J = |
| | | 6.1/6.1 Hz, 2H, NHCH₂CH₂), 2.60 (dd, J 11.9/6.7 Hz, 1H, CH(OH)CH₂NH), 1.52- |
| | | 1.75 (m, 6H, ^{c}Pe CH₂), 1.40-1.52 (m, 2H, ^{c}Pe CH₂). |
| 47p | 148-150 | (DMSO-d₆): δ 8.77 (s, 1H, NH(C═O)NHAr), 7.37 (s, 4H, C—H of bromophenyl |
| | Rₜ: 4.47 (S | ring), 6.84 (d, J = 9.3 Hz, 2H, C—H aryl-dioxy ring), 6.82 (d, J = 9.2 Hz, 2H, C—H |
| | 1b), 14.45 | aryl-dioxy ring), 6.25 (t, J = 5.4 Hz, 1H, NH(C═O)NHAr), 5.03 (br s, 1H, NH), |
| | (S 3). | 3.78-3.97 (m, 6H, CH₂OAr, ^{c}Pe CH, CH(OH), ArOCH₂), 3.61 (t, J = 4.8 Hz, |
| | | 2H, ^{c}PeOCH₂), 3.17 (dt, J = 5.8/5.8 Hz, 2H, NHCH₂CH₂), 2.71 (dd, J = 11.8/3.9 |
| | | Hz, 1H, CH(OH)CH₂NH), 2.57-2.68 (m, 3H, CH(OH)CH₂NH, NHCH₂CH₂), |
| | | 1.51-1.74 (m, 6H, ^{c}Pe CH₂), 1.40-1.51 (m, 2H, ^{c}Pe CH₂). |
| 47q | 128-130 | (DMSO-d₆): δ 7.95 (d, J = 8.4 Hz, 1H, aryl 6-H), 7.86 (s, 1H, NH(C═O)NHAr), |
| | Rₜ: 4.27 (S | 7.60 (d, J = 7.9 Hz, 1H, aryl 3-H), 7.55 (dd, J = 7.9/7.9 Hz, 1H, aryl 5-H), 7.17 |
| | 1b), 13.55 | (dd, J = 7.6/7.6 Hz, 1H, aryl 4-H), 7.08 (t, J = 5.2 Hz, 1H, NH(C═O)NHAr), 6.84 |
| | (S 3). | (s, 4H, aryl-dioxy ring), 5.04 (br s, 1H, NH), 3.77-4.02 (m, 6H, CH₂OAr, ^{c}Pe |
| | | CH, CH(OH), ArOCH₂), 3.61 (t, J = 4.9 Hz, 2H, ^{c}PeOCH₂), 3.21 (dt, J = 5.8/5.8 |
| | | Hz, 2H, NHCH₂CH₂), 2.62-2.77 (m, 4H, CH(OH)CH₂NH, NHCH₂CH₂), 1.52- |
| | | 1.75 (m, 6H, ^{c}Pe CH₂), 1.42-1.52 (m, 2H, ^{c}Pe CH₂). |
| 47r | 96-98 | (DMSO-d₆): δ 9.03 (s, 1H, NH(C═O)NHAr), 7.97 (s, 1H, aryl 2-H), 7.50 (d, J = |
| | Rₜ: 4.64 (S | 8.5 Hz, 1H, aryl 6-H), 7.44 (dd, J = 7.7/7.7 Hz, 1H, aryl 5-H), 7.21 (d, J = 7.5, |
| | 1b), 14.77 | 1H, aryl 4-H), 6.85 (d, J = 9.2 Hz, 2H, C—H aryl-dioxy ring), 6.82 (d, J = 9.3 Hz, |
| | (S 3). | 2H, C—H aryl-dioxy ring), 6.36 (t, J = 5.3 Hz, 1H, NH(C═O)NHAr), 5.13 (br s, 1H, |
| | | NH), 3.81-3.97 (m, 6H, CH₂OAr, ^{c}Pe CH, CH(OH), ArOCH₂), 3.61 (t, J = 4.9 |
| | | Hz, 2H, ^{c}PeOCH₂), 3.22 (dt, J = 5.7/5.7 Hz, 2H, NHCH₂CH₂), 2.63-2.83 (m, |
| | | 4H, CH(OH)CH₂NH, NHCH₂CH₂), 1.51-1.74 (m, 6H, ^{c}Pe CH₂), 1.41-1.51 |
| | | (m, 2H, ^{c}Pe CH₂). |
| 47s | 134-136° C. | (DMSO-d₆): δ 9.08 (s, 1H, NH(C═O)NHAr), 7.59 (d, J = 9.0 Hz, 2H, 2-H and 6- |
| | Rₜ: 4.67 (S | H of trifluoromethylphenyl ring), 7.55 (d, J = 9.1 Hz, 2H, 3-H and 5-H of |
| | 1b), 14.90 | trifluoromethylphenyl ring), 6.85 (d, J = 9.2 Hz, 2H, C—H aryl-dioxy ring), 6.82 (d, |
| | (S 3). | J = 9.3 Hz, 2H, C—H aryl-dioxy ring), 6.38 (t, J = 5.4 Hz, 1H, NH(C═O)NHAr), |
| | | 5.03 (br s, 1H, NH), 3.77-4.01 (m, 6H, CH₂OAr, ^{c}Pe CH, CH(OH), ArOCH₂), |
| | | 3.61 (t, J = 4.9 Hz, 2H, ^{c}PeOCH₂), 3.21 (dt, J = 5.8/5.8 Hz, 2H, NHCH₂CH₂), |
| | | 2.74 (dd, J = 11.8/3.8 Hz, 1H, CH(OH)CH₂NH), 2.69 (t, J = 5.9 Hz, 2H, |
| | | NHCH₂CH₂), 2.63 (dd, J = 12.0/6.6 Hz, 1H, CH(OH)CH₂NH), 1.52-1.75 (m, |
| | | 6H, ^{c}Pe CH₂), 1.38-1.52 (m, 2H, ^{c}Pe CH₂). |
| 47t | semi-solid. | (DMSO-d₆): δ 8.01 (s, 1H, NH(C═O)NHAr), 7.83 (dd, J = 7.8/1.6 Hz, 1H, aryl 6- |
| | Rₜ: 4.10 (S | H), 6.91 (t, J = 5.4 Hz, 1H, NH(C═O)NHAr), 6.85 (d, J = 9.7 Hz, 2H, aryl-dioxy |
| | 1b), 11.84 | ring), 6.82 (d, J = 9.30 Hz, 2H, aryl-dioxy ring), 6.78 (dd, J = 7.8/1.7 Hz, 1H, |
| | (S 3). | aryl 3-H), 6.73, (ddd, J = 7.7/7.3/1.2 Hz, 1H, aryl 4-H), 6.68, (ddd, J = |
| | | 7.8/7.3/1.7 Hz, 1H, aryl 5-H), 6.09 (br s, 1H, phenol), 3.80-4.00 (m, 6H |
| | | CH₂OAr, ^{c}Pe CH, CH(OH), ArOCH₂), 3.61 (t, J = 4.8 Hz, 2H, ^{c}PeOCH₂), 3.19 |
| | | (dt, J = 5.8/5.8 Hz, 2H, NHCH₂CH₂), 2.75 (dd, J = 12.1/3.9 Hz, 1H, |
| | | CH(OH)CH₂NH), 2.61-2.69 (m, 3H, NHCH₂CH₂, CH(OH)CH₂NH), 1.40-1.72 |
| | | (m, 8H, ^{c}Pe CH₂). |
| 47u | semi-solid. | (DMSO-d₆): δ 9.18 (br s, 1H, phenol), 8.47 (s, 1H, NH(C═O)NHAr), 6.98 (s, 1H, |
| | Rₜ: 3.90 (S | aryl 2-H), 6.96 (dd, J = 8.1 Hz, 1H, aryl 5-H), 6.77-6.90 (m, 4H, aryl-dioxy |
| | 1b), 11.02 | ring), 6.71 (d, J = 7.8 Hz, 1H, aryl 6-H), 6.28 (dd, J = 7.8/1.8 Hz, 1H, aryl 4-H), |
| | (S 3). | 6.16 (t, J = 5.2 Hz, 1H, NH(C═O)NHAr), 3.75-4.02 (m, 6H, CH₂OAr, ^{c}PeCH, |
| | | CH(OH), ArOCH₂), 3.61 (t, J = 4.8 Hz, 2H, ^{c}PeOCH₂), 3.15 (dt, J = 5.7/5.7 Hz, |
| | | 2H, NHCH₂CH₂), 2.70 (dd, J = 12.0/3.7 Hz, 1H, CH(OH)CH₂NH), 2.54-2.67 |
| | | (m, 3H, CH(OH)CH₂NH, NHCH₂CH₂), 1.38-1.74 (m, 8H, ^{c}Pe CH₂). |
| 47v | 135-138 | (DMSO-d₆): δ 8.91 (br s, 1H, phenol), 8.20 (s, 1H, NH(C═O)NHAr), 7.13 (d, J = |
| | Rₜ: 3.17 (S | 8.8 Hz, 2H, aryl C—H ortho to urea), 6.84 (s, 4H, aryl-dioxy ring), 6.62 (d, J = 8.8 |
| | 1b), 10.30 | Hz, 2H, aryl C—H ortho to phenol), 6.02 (t, J = 5.4 Hz, 1H, NH(C═O)NHAr), 4.96 |
| | (S 3). | (br s, 1H, NH), 3.79-3.97 (m, 6H, CH₂OAr, ^{c}Pe CH, CH(OH), ArOCH₂), 3.61 |
| | | (t, J = 4.8 Hz, 2H, ^{c}PeOCH₂), 3.12-3.16 (m, 2H, NHCH₂CH₂), 2.56-2.71 (m, |
| | | 4H, CH(OH)CH₂NH, NHCH₂CH₂), 1.54-1.69 (m, 6H, ^{c}Pe CH₂), 1.42-1.51 |
| | | (m, 2H, ^{c}Pe CH₂). |
| 47w | Rₜ: 2.30 (s 4), 2.44 (s 5) | |
| 47x | 134.5-137.5 Rₜ: 2.24 (s 4), 2.42 (s 5) | |
| 47y | 126-128 Rₜ: 2.21 (s 4), 2.40 (s 5) | |
| 47z | 117.5-119; Rₜ: 2.21 (s 4), 2.39 (s 5) | |
| 47aa | 185-188; Rₜ: 2.11 (s 4), 2.30 (s 5) | |
| 47bb | 183-186; Rₜ: 2.08 (s 4), 2.24 (s 5); 1H nmr data similar to other compounds in table | |
| 48 | 155-157 | (CDCl₃): δ 7.29 (dd, J = 8.7/7.5 Hz, 2H, phenoxy 3-H and 5-H), 6.96 (dd, J = |
| | Rₜ: 4.47 (S | 7.4/7.4 Hz, 1H, phenoxy 4-H), 6.91 (d, J = 7.7 Hz, 2H, phenoxy 2-H and 6-H), |
| | 1b), 12.64 | 6.80-6.86 (m, 4H, aryl-dioxy ring), 3.91-4.11 (m, 8H, OCH₂CH₂OAr, |
| | (S 3). | NHCH₂CH₂OAr, ArOCH₂CH, CH(OH), ^{c}Pe CH), 3.72 (t, J = 5.2 Hz, 2H, |
| | | ^{c}PeOCH₂), 3.06 (t, J = 5.1 Hz, 2H, NHCH₂CH₂OAr), 2.94 (dd, J = 12.1/3.9 Hz, |
| | | 1H, CH(OH)CH₂NH), 2.84 (dd, J = 12.2/7.9 Hz, 1H, CH(OH)CH₂NH), 1.44- |
| | | 1.84 (m, 8H, ^{c}Pe CH₂). |
| 49 | Rₜ: 4.20 (S | ¹H NMR (DMSO-d₆): δ 8.08 (br s, 1H, amide NH), 7.17-7.32 (m, 5H, phenyl |
| | 1b), 12.14 | CH), 6.85 (s, 4H, aryl-dioxy CH), 3.98 (t, J = 4.8 Hz, 2H, CH₂OAr), 3.90-3.96 |
| | (S 3). | (m, 1H, ^{c}Pe CH), 3.77-3.90 (m, 3H, ArOCH₂, CH(OH)), 3.62 (t, J = 4.9 Hz, |
| | | 2H, ^{c}PeOCH₂), 3.40 (s, 2H, C═OCH₂), 3.12-3.20 (m, 2H, CH₂NHC═O), 2.56- |
| | | 2.77 (m, 4H, CH(OH)CH₂NH, CH₂NHCH₂), 1.41-1.76 (m, 8H, ^{c}Pe CH₂). |
| 50 | Rₜ: 4.30 (S | (DMSO-d₆): δ 9.83 (s, 1H, amide NH), 8.28 (br s, 2H, NH₂⁺), 7.58 (d, J = 7.6 |
| | 1b), 12.44 | Hz, 2H, phenyl 2-H and 6-H), 7.27 (dd, J = 7.8/7.8 Hz, 2H, phenyl 3-H and 5-H), |
| | (S 3). | 7.01 (dd, J = 7.4/7.4 Hz, 1H, phenyl 4-H), 6.84 (s, 4H, aryl-dioxy CH), 3.98 (t, J = |
| | | 4.8 Hz, 2H, CH₂OAr), 3.78-3.96 (m, 4H, ArOCH₂, CH(OH), ^{c}Pe CH), 3.62 (t, |
| | | J = 4.9 Hz, 2H, ^{c}PeOCH₂), 2.76 (dd, J = 11.9/4.1 Hz, 1H, CH(OH)CH₂), 2.61- |
| | | 2.69 (m, 3H, CH(OH)CH₂, NHCH₂), 2.36 (t, J = 7.4 Hz, 2H, CH₂C═O), 1.72- |
| | | 1.82 (m, 2H, CH₂CH₂CH₂), 1.41-1.72 (m, 8H, ^{c}Pe CH₂). |
| 51 | Rₜ: 4.47 (S | (DMSO-d₆): δ 9.64 (s, 1H, carbamate NH), 7.46 (d, J = 7.3 Hz, 2H, 2-H and 6-H |
| | 1b), 12.64 | phenyl ring, 7.26 (dd, J = 7.3/7.3 Hz, 2H, 3-H and 5-H phenyl ring), 6.99 (dd, J = |
| | (S 3). | 7.3/7.3 Hz, 1H, 4-H phenyl ring), 6.84 (s, 4H, aryl-dioxy CH), 4.99 (br s, 1H, |
| | | OH), 4.14 (t, J = 5.7 Hz, 2H, CH₂OC═O), 3.76-4.00 (m, 6H, CH₂OAr, ^{c}Pe CH, |
| | | CH(OH), ArOCH₂), 3.61 (t, J = 4.8 Hz, 2H, ^{c}PeOCH₂), 2.82 (t, J = 5.7 Hz, 2H, |
| | | NHCH₂CH₂O), 2.71 (dd, J = 11.9/3.8 Hz, 1H, CH(OH)CH₂), 2.61 (dd, J = |
| | | 11.5/6.1 Hz, 1H, CH(OH)CH₂), 1.41-1.75 (m, 8H, ^{c}Pe CH₂). |
| 52 | Rₜ: 4.25 (S | (DMSO-d₆): δ 9.66 (br s, 1H, NH(C═S)NHAr), 7.73 (br s, 1H, NH(C═S)NHAr), |
| | 1b), 12.02 | 7.42 (d, J = 8.1 Hz, 2H, 2-H and 6-H phenyl ring), 7.29 (dd, J = 7.4/7.4 Hz, 2H, |
| | (S 3). | 3-H and 5-H phenyl ring), 7.08 (dd, J = 7.4/7.4 Hz, 1H, 4-H phenyl ring), 6.84 |
| | | (s, 4H, aryl-dioxy ring), 4.99 (br s, 1H, OH), 3.75-4.03 (m, 6H, CH₂OAr, ^{c}Pe |
| | | CH, CH(OH), ArOCH₂), 3.62 (t, J = 4.9 Hz, 2H, ^{c}PeOCH₂), 3.55 (br s, 2H, |
| | | NHCH₂CH₂), 2.75 (t, J = 6.3 Hz, 2H, NHCH₂CH₂), 2.67-2.73 (m, 1H, |
| | | CH(OH)CH₂NH), 2.61 (dd, J = 11.8/5.9 Hz, 1H, CH(OH)CH₂NH), 1.39-1.76 |
| | | (m, 8H, ^{c}Pe CH₂). |
| 53 | Rₜ: 4.39 (S | (DMSO-d₆): δ 7.29-7.43 (m, 5H, C—H, phenyl ring), 6.84 (s, 4H, aryl-dioxy |
| | 1b), 12.05 | ring), 4.94 (br s, 1H, OH), 4.33 (s, 2H, SO₂CH₂), 3.74-4.02 (m, 6H, CH₂OAr, |
| | (S 3). | ^{c}Pe CH, CH(OH), ArOCH₂), 3.62 (t, J = 4.9 Hz, 2H, ^{c}PeOCH₂), 2.97 (t, J = 6.5 |
| | | Hz, 2H, CH₂NHSO₂), 2.52-2.69 (m, 4H, NHCH₂CH₂, CH(OH)CH₂NH), 1.40- |
| | | 1.77 (m, 8H, ^{c}Pe CH₂). |
| 54 | semi-solid. | (DMSO-d₆): δ 9.30 (br s, 1H, phenolic OH), 8.60 (s, 1H, NH(C═O)NHAr), 7.32- |
| | Rₜ: 3.82 (S | 7.42 (m, 1H, phenylurea C—H), 6.75-6.92 (m, 6H, C—H aryldioxy ring, |
| | 1b). | phenylurea C—H), 6.26 (t, J = 5.6 Hz, 1H, NH(C═O)NHAr), 3.75-4.04 (m, 6H, |
| | | CH₂OAr, ^{c}Pe CH, CH(OH), ArOCH₂), 3.61 (t, J = 4.8 Hz, 2H, ^{c}PeOCH₂), 3.20- |
| | | 3.27 (m, 2H, NHCH₂CH₂), 2.72-2.94 (m, 4H, CH(OH)CH₂NH, NHCH₂CH₂), |
| | | 1.52-1.75 (m, 6H, ^{c}Pe CH₂), 1.41-1.52 (m, 2H, ^{c}Pe CH₂). |
| 54a | 113-115 | (DMSO-d₆): δ 8.90 (s, 1H, NH(C═O)NHAr), 7.63 (ddd, J = 13.8/7.9/2.6 Hz, 1H, |
| | Rₜ: 4.65 (S | aryl 2-H), 7.26 (ddd, J = 10.4/9.2/9.2 Hz, 1H, aryl 5-H), 6.96-7.06 (m, 1H, aryl |
| | 1b), 13.70 | 6-H), 6.84 (s, 4H, C—H aryl-dioxy ring), 6.32 (t, J = 5.2 Hz, 1H, NH(C═O)NHAr), |
| | (S 3). | 5.11 (br s, 1H, OH), 3.76-4.03 (m, 6H, CH₂OAr, ^{c}Pe CH, CH(OH), ArOCH₂), |
| | | 3.61 (t, J = 4.6 Hz, 2H, ^{c}PeOCH₂), 3.14-3.24 (m, 2H, NHCH₂CH₂), 2.59-2.81 |
| | | (m, 4H, CH(OH)CH₂NH, NHCH₂CH₂), 1.52-1.75 (m, 6H, ^{c}Pe CH₂), 1.41- |
| | | 1.52 (m, 2H, ^{c}Pe CH₂). |

| | |
|---|---|
| 54b | Rₜ: 2.17 (s 4), 2.49 (s 5) |
| 54c | Rₜ: 2.02 (s 4), 2.34 (s 5); 1H-nmr data similar to other compounds in table |

### General Procedure for Synthesis of Phenyl Substituted N-(2-(3-(4-(2-(cyclopentyloxy)ethoxy)phenoxy)-2-hydroxypropylamino)ethyl)benzamides (54d-i)

Epoxide opening of 41c with the appropriate 22c-h, was carried out as described for 47t. Purification was achieved via PLC (eluent NH₃/MeOH/DCM 1:5:94).

### N-(2-(3-(4-(2-(cyclopentyloxy)ethoxy)phenoxy)-2-hydroxypropylamino)ethyl)-2-(3-hydroxyphenyl)acetamide (54j)

Epoxide opening of 41c with 18b was carried out as described for 47t. Purification via PLC (eluent NH₃/MeOH/DCM 1:10:89). Table 18 lists ¹H NMR spectral data for compounds from FIG. 4:

| | m.p/° C.; | |
|---|---|---|
| Cpd | HPLC | ¹H NMR |
| 54d | Rₜ: 3.57 (S | (DMSO-d₆): δ 8.76-8.87 (m, 1H, NH(C═O)), 7.84 (dd, J = 7.9/1.6 Hz, 1H, |
| | 1b) | hydroxyaryl 6H), 7.38 (ddd, J = 7.8/7.8/1.5 Hz, 1H, hydroxyaryl 4H), 6.84- |
| | 12.62 (S | 6.91 (m, 2H, hydroxyaryl 3H, 5H), 6.83 (s, 4H, aryl-dioxy CH), 3.76-4.00 (m, |
| | 3). | 6H, CH₂OAr, CH(OH), ArOCH₂, ^{c}Pe CH), 3.61 (t, J = 4.7 Hz, 2H, ^{c}PeOCH₂), |
| | | 3.39 (dt, J = 6.1/5.5 Hz, 2H, CH₂NH(C═O)), 2.75 (t, J = 6.4 Hz, 2H, |
| | | NHCH₂CH₂NH), 2.72 (dd, J = 12.2/4.4 Hz, 1H, CH(OH)CH₂NH), 2.67 (dd, J = |
| | | 11.6/6.1 Hz, 1H, CH(OH)CH₂NH), 1.42-1.73 (m, 8H, ^{c}Pe CH₂). |
| 54e | Rₜ: 4.02 (S | (DMSO-d₆): δ 9.60 (br s, 1H, phenol OH), 8.33 (t, J = 5.6 Hz, 1H, NH(C═O)), |
| | 1b), 11.23 | 7.19-7.29 (m, 3H, hydroxyaryl 2H, 5H, 6H), 6.90 (ddd, J = 7.3/2.0/2.0 Hz, 1H, |
| | (S 3). | aryl 4H), 6.83 (s, 4H, aryl-dioxy CH), 3.77-4.00 (m, 6H, CH₂OAr, CH(OH), |
| | | ArOCH₂, ^{c}Pe CH), 3.61 (t, J = 5.0 Hz, 2H, ^{c}PeOCH₂), 3.35 (dt, J = 6.1/5.8 Hz, |
| | | 2H, CH₂NH(C═O)), 2.72-2.80 (m, 3H, NHCH₂CH₂NH, CH(OH)CH₂NH), |
| | | 2.65 (dd, J = 12.0/6.4 Hz, 1H, CH(OH)CH₂NH), 1.42-1.73 (m, 8H, ^{c}Pe CH₂). |
| 54f | Rₜ: 3.57 (S | (DMSO-d₆): δ 8.19-8.34 (m, 2H, phenol OH, NH(C═O)), 7.71 (d, J = 8.6 Hz, |
| | 1b), 10.47 | 2H, hydroxyaryl 2H, 6H), 6.84 (s, 4H, aryl-dioxy CH), 6.78 (d, J = 8.6 Hz, 2H, |
| | (S 3). | hydroxyaryl 3H, 5H), 3.77-4.00 (m, 6H, CH₂OAr, CH(OH), ArOCH₂, ^{c}Pe |
| | | CH), 3.62 (t, J = 4.7 Hz, 2H, ^{c}PeOCH₂), 3.37 (dt, J = 6.0/5.7 Hz, 2H, |
| | | CH₂NH(C═O)), 2.76-2.85 (m, 3H, NHCH₂CH₂NH, CH(OH)CH₂NH), 2.70 |
| | | (dd, J = 12.0/7.0 Hz, 1H, CH(OH)CH₂NH), 1.42-1.73 (m, 8H, ^{c}Pe CH₂). |
| 54g | 75-76 | (DMSO-d₆): δ 8.22 (br s, 1H, NH(C═O)), 7.62 (ddd, J = 7.5/7.5/1.8 Hz, 1H, |
| | Rₜ: 3.88 (S | fluoroaryl-6H), 7.46-7.56 (m, 1H, fluoroaryl 4H), 7.22-7.31 (m, 2H, |
| | 1b), 11.94 | fluoroaryl 3H, 5H), 6.83 (s, 4H, aryl-dioxy CH), 4.91 (br s, 1H, OH), 3.77- |
| | (S 3). | 4.00 (m, 6H, CH₂OAr, CH(OH), ArOCH₂, ^{c}Pe CH), 3.62 (t, J = 4.7 Hz, 2H, |
| | | ^{c}PeOCH₂), 3.31-3.36 (m, 11H, CH₂NH(C═O) under water peak), 2.65- |
| | | 2.74 (m, 3H, NHCH₂CH₂NH, CH(OH)CH₂NH), 2.59 (dd, J = 11.9/6.2 Hz, 1H, |
| | | CH(OH)CH₂NH), 1.42-1.73 (m, 8H, ^{c}Pe CH₂). |
| 54h | 85-86 | (DMSO-d6): δ 8.50 (t, J = 5.3 Hz, 1H, NH(C═O)), 7.69 (ddd, J = 7.7/1.2/1.2 |
| | Rt: 3.95 | Hz, 1H, fluoroaryl 6H), 7.63 (ddd, J = 10.1/2.5/1.6 Hz, 1H, fluoroaryl 2H), |
| | (S 1b), | 7.51 (ddd, J = 7.9/7.9/5.7 Hz, 1H, fluoroaryl 5H), 7.36 (dddd, J = |
| | 12.57 (S | 8.8/8.8/2.6/0.9, fluoroaryl 4H), 6.83 (s, 4H, ary-dioxy CH), 4.91 (br s, 1H, OH), |
| | 3). | 3.76-4.00 (m, 6H, CH2OAr, CH(OH), ArOCH2, cPe CH), 3.62 (t, J = 4.8 Hz, |
| | | 2H, cPeOCH2), 3.35 (dt, J = 6.1/5.9 Hz, 2H, CH2NH(C═O)), 2.71 (t, 2H, J = |
| | | 6.4 Hz, NHCH2CH2NH), 2.65-2.71 (m, 1H, CH(OH)CH2NH), 2.60 (dd, J = |
| | | 11.7/6.1 Hz, 1H, CH(OH)CH2NH), 1.42-1.73 (m, 8H, cPe CH2). |
| 54i | 99-100 | (DMSO-d6): δ 8.42 (t, J = 5.2 Hz, 1H, NH(C═O)), 7.90 (dd, J = 8.9/5.4 Hz, |
| | Rt: 3.93 | 2H, fluoroaryl 2H, 6H), 7.27 (dd, J = 9.1/9.1 Hz, 2H, fluoroaryl 3H, 5H), 6.83 |
| | (S 1b), | (s, 4H, aryl-dioxy CH), 4.90 (br s, 1H, OH), 3.76-4.00 (m, 6H, CH2OAr, |
| | 12.59 (S | CH(OH), ArOCH2, cPe CH), 3.61 (t, J = 4.8 Hz, 2H, cPeOCH2), 3.34 (dt, J = |
| | 3). | 6.5/5.8 Hz, 2H, CH2NH(C═O)), 2.65-2.74 (m, 3H, NHCH2CH2NH, |
| | | CH(OH)CH2NH), 2.59 (dd, J = 12.0/6.3 Hz, 1H, CH(OH)CH2NH), 1.42-1.73 |
| | | (m, 8H, cPe CH2). |
| 54j | Rt: 3.88 | (DMSO-d6): δ 9.26 (s, 1H, phenol O—H), 7.97 (t, J = 5.5 Hz, 1H, NH(C═O)), |
| | (S 1b), | 7.04 (dd, J = 7.8/7.8 Hz, 1H, hydroxyaryl 5-H), 6.84 (s, 4H, aryl-dioxy ring), |
| | 11.27 (S | 6.54-6.72 (m, 3H, hydroxyaryl 2H, 4H, 6H), 4.96 (s, 1H, alcohol OH), 3.98 |
| | 3) | (t, J = 4.9 Hz, 2H, CH2CH2OAr), 3.75-3.96 (m, 4H, ArOCH2CH(OH), |
| | | CH(OH), cPe CH), 3.61 (t, J = 4.8 Hz, 2H, OCH2CH2OAr), 3.29 (s, 2H, |
| | | NH(C═O)CH2), 3.13 (dt, J = 5.8/5.8 Hz, 2H, NHCH2CH2NH), 2.54-2.74 |
| | | (m, 4H, CH(OH)CH2NH, NHCH2CH2NH(C═O)), 1.40-1.77 (m, 8H, cPe |
| | | CH2). |

### tert-Butyl 2-(benzylamino)ethylcarbamate (56)

55 (15.861 g, 105.58 mmol) was dissolved in MeOH at rt. To this stirred solution was added 2-(tert-butoxycarbonyloxyimino)-2-phenylacetonitrile (26.00 g, 105.58 mmol, 1 eq), in portions, allowing dissolution before next addition. The resulting yellow solution was stirred at rt for 3 days (over weekend) before removal of all volatiles under reduced pressure. The crude residue was dissolved in EtOAc (300 mL) before washing with aq. 1M NaOH (2×100 mL). The combined aqueous layers were again washed with EtOAc (100 mL). Combining and concentrating the organic portions gave 29.8 g of crude product. This was further purified by FCC (eluent DCM/PE 1:1 to load column, continued until impurities wash off, then 100% DCM, followed MeOH/DCM 1:10). This gave 25.3 g (96%) of pale yellow oil.

### tert-Butyl 2-(benzyl(3-(4-(2-(cyclopentyloxy)ethoxy)phenoxy)-2-hydroxypropyl)amino)ethylcarbamate (57)

41c (5.00 g, 17.96 mmol) and 56 (4.497 g, 17.96 mmol, 1 eq) were dissolved in EtOH (30 mL) and a few drops of water added, before splitting the mixture into 2×30 mL MW vessels. Each mixture was heated at 100° C. for 30 mins in the MW reactor (dynamic program with maximum pressure 250 psi, maximum power 300W). Concentration of the reaction mixture gave approximately 10 g of crude residue. This was purified by FCC (eluent PE/DCM 1:1 to prime/load the column, with the gradient increasing to 100% DCM over 5CV, 100% DCM for a further 3 CV and then raise to DCM/MeOH 99:1 over 1 CV, then to 95:5 over 3 CV, holding at this concentration to elute the desired product). This gave 7.30 (77%) g of white crystalline solid.

### 1-((2-Aminoethyl)(benzyl)amino)-3-(4-(2-(cyclopentyloxy)ethoxy)phenoxy)propan-2-ol dihydrochloride (58)

57 (606 mg, 1.15 mmol) was dissolved in DCM (5 mL) with stirring before adding 4M HCl/Dioxane (5 mL) and 1 drop of water. The mixture was stirred for 15 minutes before diluting with PE and collecting the precipitate by filtration (vacuum) to give 457 mg (79%) of beige amorphous solid.

### General Procedure for Synthesis of 1-(2-(benzyl(3-(4-(2-(cyclopentyloxy)ethoxy)phenoxy)-2-hydroxypropyl)amino)ethyl)-3-(nitrophenyl)ureas (59a-b)

57 (100 mg, 0.20 mmol and TEA (0.058 mL, 0.42 mmol, 2.1 eq) were dissolved in a mixture of DCM/DMF (1:1, 1 mL) in three Radley's tubes. The appropriate nitro-phenylisocyanate was added as a solution in DMF (0.5 mL) with washings of a further 0.5 mL of solvent. The mixtures were stirred at rt over the weekend. To each mixture was added sat. aq. NaHCO₃ (30 mL), before extracting with DCM (3×10 mL). The combined organic layers were then further washed with brine (10 mL). After concentration of the organic layer under reduced pressure, each crude residue was further purified by FCC (eluent 1N NH₃ in MeOH/DCM 1:99 to wash out impurities, then up to 5:95 to elute).

### General Procedure for Synthesis of 1-(aminophenyl)-3-(2-(3-(4-(2-(cyclopentyloxy)ethoxy)phenoxy)-2-hydroxypropylamino)ethyl)ureas (60a-c)

59a-b, 47w were dissolved in MeOH (2 mL) and 10% Pd/C added under nitrogen. MeOH (1 mL) was used to wash out starting material container, before adding water/AcOH (2:1, 1.2 mL). The tubes were hydrogenated on a Radley's carousel at atmospheric temperature and pressure over the weekend. Each mixture was passed through a bed of celite with rinsings of MeOH, before concentration of the filtrate. The desired products were purified by FCC (eluent 1N NH₃ in MeOH/DCM 0.5:99.5 to 20:80 over 10 CV). Yield 17-53%. HPLC (S 4; S 5): 60a 2.07; 2.20. 60b 1.92; 2.04. 60c 1.81; 1.98. 1H-nmr very similar to other urea compounds.

### 1-(2-Aminoethylamino)-3-(4-(2-(cyclopentyloxy)ethoxy)phenoxy)propan-2-ol dihydrochloride (61)

57 (2.0 g) was dissolved in a mixture of MeOH/water/AcOH (7:2:1, 20 mL) with 10% Pd/C (200 mg) and hydrogenated on a Parr hydrogenator at 50 psi overnight to give the de-benzylated intermediate (clean and complete conversion by TLC). The reaction was repeated with a further 4.242 g of 57, using 40 mL of the same solvent mixture and 420 mg of catalyst. The reaction was complete in 5 hours. The combined products were concentrated under reduced pressure and combined to give the intermediate as the Boc-protected acetate salt. This was purified and converted to the free amine by passing through a silica plug (eluent 1M NH₃ in MeOH/DCM 5:95 to 20:80), yielding 5.918 g of intermediate. After dissolving in 20 mL of DCM, with vigorous stirring at rt, an equivalent volume of 4M HCl/Dioxane was added, and the mixture allowed to stir for 1 hour at which point an off-white precipitate had formed. The mixture was diluted with excess PE and the precipitate collected over a sintered funnel and further washed with PE, before drying in a dessicator under vacuum, to give the desired product as 3.717 g (77%) of the dihydrochloride salt.

### General Procedure for Selective Coupling 61 to Carboxylic Acids (62a-d,f-v)

61 (75-100 mg), HBTU (1.1 eq) and appropriate unsubstituted, mono-substituted or di-substituted benzoic acid, or mono-substituted phenylacetic acid (1 eq) were weighed into a vessel, before dissolving in DCM (5-7 mL). TEA (3.1 eq) was added, and the mixture stirred overnight at rt. All volatiles were removed under reduced pressure before being purified by FCC (eluent initially 100% DCM to load/prime column, then a gradient of 1M NH₃ MeOH/DCM (1:99 to 15:85 or 20:80 over 10 CV depending TLC analysis). The isolated target compound was then freeze-dried to give amorphous or hygroscopic solids (yields: 11-90%).

### 4-(2-(3-(4-(2-(Cyclopentyloxy)ethoxy)phenoxy)-2-hydroxypropylamino)ethylcarbamoyl)phenyl acetate (62e)

The title compound was synthesised according to the general procedure for selective coupling of 61 to carboxylic acids. Prior to FCC purification, the reaction mixture was diluted with DCM (20 mL) and washed with aq. Sat NaHCO₃/brine (1:1) (40 mL). The aqueous phase was extracted with a further 20 mL of DCM. The combined organic extracts were concentrated. After purification, 40 mg (33%) of white amorphous solid was obtained.

### 4-(2-(2-(3-(4-(2-(Cyclopentyloxy)ethoxy)phenoxy)-2-hydroxypropylamino)ethylamino)-2-oxoethyl)phenyl acetate (62w)

The title compound was synthesised according to the procedure described for 62e. After purification, 67 mg (54%), of white amorphous solid was obtained.

Table 20 lists the ¹H NMR spectral data for selected compounds from Scheme 5:

| | m.p/° C.; | |
|---|---|---|
| Cpd | HPLC | ¹H NMR |
| 62a | 149-152 | (DMSO-d₆) δ 8.79 (br t, J = 5.3 Hz, 1H), 7.87-7.95 (m, 2H), 7.44-7.59 |
| | Rₜ: 1.92 (s | (m, 3H), 6.87 (s, 4H), 5.80-5.87 (br m, 1H), 4.13-4.24 (br m, 1H) 3.85- |
| | 4), 2.04 (s 5) | 4.02 (m, 5H), 3.58-3.67 (m, 4H), 3.13-3.27 (m, 3H), 2.99-3.09 (m, |
| | | 1H), 1.39-1.75 (m, 8H) |
| 62b | 107-109 | (DMSO-d₆) δ 8.06-8.16 (br m, 1H), 7.45 (d, J = 7.7 Hz, 1H), 7.12 (dd, J = |
| | Rₜ: 2.17 (s | 7.5 Hz, 1H), 6.83 (s, 4H), 6.68 (d, J = 7.8 Hz, 1H), 6.49 (dd, J = 7.1 Hz, |
| | 4), 2.35 (s 5) | 1H), 6.35 (br s, 2H), 4.90 (br s, 1H), 3.75-4.03 (m, 6H), 3.58-3.66 (m, |
| | | 2H), 3.25-3.33 (m, 2H), 2.64-2.73 (m, 3H), 2.59 (dd, J = 11.5/5.8 Hz, |
| | | 1H), 1.40-1.76 (m, 8H) |
| 62c | 105-107.5 | (DMSO-d₆) δ 8.49 (br t, J = 5.3 Hz, 1H), 7.04-7.13 (m, 2H), 7.01 (d, J = |
| | Rₜ: 2.04 (s | 7.6 Hz, 1H), 6.87 (s, 4H), 6.70 (d, J = 8.8, 1H), 5.78 (br s, 1H), 5.23 (br s, |
| | 4), 2.21 (s 5) | 2H), 4.11-4.21 (m, 1H), 3.85-4.02 (m, 5H), 3.59-3.65 (m, 2H), 3.56 |
| | | (dt, J = 5.7/5.7 Hz, 2H) 3.14-3.21 (m, 1H), 3.11 (t, J = 5.9 Hz, 2H), 2.96- |
| | | 3.04 (m, 1H), 1.39-1.77 (m, 8H) |
| 62d | 67-69 | (DMSO-d₆) δ 8.28 (br t, J = 5.2 Hz, 1H), 7.61 (d, J = 8.1 Hz, 2H), 6.86 (s, |
| | Rₜ: 2.07 (s | 4H), 6.54 (d, J = 8.1 Hz, 2H), 5.64 (s, 2H), 4.08-4.19 (m, 1H), 3.98 (t, J = |
| | 4), 2.23 (s 5) | 4.6 Hz, 2H), 3.85-3.96 (m, 3H), 3.62 (t, J = 4.6 Hz, 2H), 3.52 (dt, J = |
| | | 5.7/5.7 Hz, 2H), 3.04-3.17 (m, 3H), 2.91-3.00 (m, 1H), 1.40-1.76 (m, 8H) |
| 62e | Rₜ: 2.10 (s 4), 2.26 (s 5) | |
| 62f | Decomp. | (DMSO-d₆) δ 8.26 (t, J = 5.4 Hz, 1H), 7.42 (d, J = 1.8 Hz, 1H), 7.34 (dd, J = |
| | 160 | 8.2/1.8 Hz, 1H), 6.84 (s, 4H), 6.80 (d, J = 8.2 Hz, 1H), 5.11 (br s, 1H), 3.97 |
| | Rₜ: 2.12 (s | (t, J = 4.8 Hz, 2H), 3.78-3.95 (m, 7H), 3.62 (t, J = 4.8 Hz, 2H), 3.37 (dt, J = |
| | 4), 2.27 (s 5) | 6.0/6.0 Hz, 2H), 2.75-2.86 (m, 3H), 2.69 (dd, J = 12.0/7.0 Hz, 1H), 1.39- |
| | | 1.75 (m, 8H) |
| 62g | 122-124 | (DMSO-d₆) δ 8.00 (br s, 1H), 7.21-7.36 (m, 2H), 7.06-7.18 (m, 2H), |
| | Rₜ: 2.21 | 6.84 (s, 4H), 4.90 (br s, 1H), 3.73-4.06 (m, 6H), 3.58-3.65 (m, 2H), 3.46 |
| | (s4), 2.37 | (s, 2H), 3.10-3.19 (m, 2H), 2.52-2.70 (m, 4H) 1.40-1.73 (m, 8H) |
| | (s5) | |
| 62h | 116.5-118 | (DMSO-d₆) δ 8.02 (br s, 1H), 7.31 (dd, J = 14.5/7.4 Hz, 1H) 6.98-7.14 (m, |
| | Rₜ: 2.22 (s | 3H), 6.85 (s, 4H), 4.89 (br s), 3.75-4.02 (m, 6H), 3.62 (t, J = 4.5 Hz, 2H), |
| | 4), 2.39 (s 5) | 3.43 (s, 2H), 3.13 (dt, J = 5.6/5.6 Hz, 2H), 2.52-2.71 (m, 4H) 1.39- |
| | | 1.76 (m, 8H) |
| 62i | 114.5-115.5 | (DMSO-d₆) δ 7.99 (br t, J = 5.2 Hz, 1H), 7.27 (dd, J = 8.5/5.7 Hz, 2H) 7.09 |
| | Rₜ: 2.23 (s | (dd, J = 8.9/8.9 Hz, 2H), 6.84 (s, 4H), 4.91 (br s, 1H), 3.75-4.01 (m, 6H), |
| | 4), 2.38 (s 5) | 3.62 (t, J = 4.8 Hz, 2H), 3.39 (s, 2H), 3.12 (dt, J = 6.1/6.1 Hz, 2H), 2.65 |
| | | (dd, J = 11.8/4.1 Hz, 1H), 2.52-2.62 (m, 3H) 1.39-1.75 (m, 8H) |
| 62j | 113.5-114.5 | (DMSO-d₆) δ 7.98 (br s, 1H), 7.20-7.44 (m, 4H), 6.84 (s, 4H), 4.89 (br s, |
| | Rₜ: 2.20 (s | 1H), 3.75-4.02 (m, 6H), 3.59-3.65 (m, 2H), 3.56 (s, 2H), 3.15 (dt, J = |
| | 4), 2.39 (s 5) | 5.8/5.8 Hz, 2H), 2.52-2.71 (m, 4H), 1.40-1.74 (m, 8H) |
| 62k | 117-118 | (DMSO-d₆) δ 8.03 (br s, 1H), 7.24-7.35 (m, 3H), 7.20 (d, J = 6.9 Hz, 1H), |
| | Rₜ: 2.287 (s | 6.84 (s, 4H), 4.89 (br s, 1H), 3.75-4.02 (m, 6H), 3.62 (t, J = 4.5 Hz, 2H), |
| | 4), 2.45 (s 5) | 3.42 (s, 2H), 3.13 (dt, J = 5.9/5.9 Hz, 2H), 2.65 (dd, J = 12.0/3.4 Hz, 1H), |
| | | 2.52-2.62 (m, 3H), 1.40-1.74 (m, 8H) |
| 62l | 107-109 | (DMSO-d₆) δ 8.01 (br s, 1H), 7.33 (d, J = 6.8 Hz, 2H), 7.26 (d, J = 7.0 Hz, |
| | Rₜ: 2.29 (s | 2H), 6.85 (s, 4H), 4.90 (br s, 1H), 3.74-4.02 (m, 6H), 3.58-3.65 (br t, |
| | 4), 2.46 (s 5) | 2H) 3.40 (s, 2H), 3.12 (dt, J = 5.8/5.7 Hz, 2H), 2.52-2.72 (m, 4H) 1.40- |
| | | 1.73(m, 8H) |
| 62m | 120.5-121.5 | (DMSO-d₆) δ 8.00 (br s, 1H), 7.67 (d, J = 7.7 Hz, 1H), 7.60 (dd, J = |
| | Rₜ: 2.29 (s | 7.48/7.48 Hz, 1H), 7.40 -7.48 (m, 2H), 6.84 (s, 4H), 4.89 (br s, 1H), 3.75- |
| | 4), 2.47 (s 5) | 4.01 (m, 6H), 3.58-3.66 (m, 4H), 3.15 (dt, J = 5.8 Hz, 2H), 2.65 (dd, J = |
| | | 11.2/3.4 Hz, 1H), 2.53-2.62 (m, 3H) 1.40-1.76 (m, 8H) |
| 62n | 111.5-112.5 | (DMSO-d₆) δ 8.08 (br s, 1H), 7.48 -7.63 (m, 4H), 6.84 (s, 4H), 4.89 (br s, |
| | Rₜ: 2.33 (s | 1H), 3.75-4.00 (m, 6H), 3.61 (t, J = 5.0 Hz, 2H), 3.52 (s, 2H), 3.13 (dt, J = |
| | 4), 2.53 (s 5) | 5.9/5.9 Hz, 2H), 2.52-2.68 (m, 4H) 1.38-1.74 (m, 8H) |
| 62o | 132-133 | (DMSO-d₆) δ 8.09 (br s, 1H), 7.64 (d, J = 8.0 Hz, 2H), 7.47 (d, J = 8.0 Hz, |
| | Rₜ: 2.33 (s | 2H), 6.84 (s, 4H), 4.90 (br s, 1H), 3.75-4.01 (m, 6H), 3.61 (t, J = 4.7 Hz, |
| | 4), 2.54 (s 5) | 2H), 3.52 (s, 2H), 3.13 (dt, J = 5.9/5.9 Hz, 2H), 2.52-2.70 (m, 4H) 1.40- |
| | | 1.73 (m, 8H) |
| 62p | 119-120.5 | (DMSO-d₆) δ 7.88 (t, J = 5.1 Hz, 1H), 7.05-7.20 (m, 4H), 6.84 (s, 4H), |
| | Rₜ: 2.25 (s | 4.89 (br s, 1H), 3.74-4.03 (m, 6H), 3.62 (t, J = 4.7 Hz, 2H) 3.42 (s, 2H), |
| | 4), 2.40 (s 5) | 3.14 (dt, J = 6.0/6.0 Hz, 2H), 2.65 (dd, J = 11.8/3.7 Hz, 1H), 2.52-2.62 |
| | | (m, 3H), 2.23 (s, 3H) 1.40-1.76 (m, 8H) |
| 62q | Rₜ: 2.24 (s | (DMSO-d₆) δ 8.56-8.66 (br m, 1H), 7.16 (dd, J = 7.5/7.5 Hz, 1H), 7.04- |
| | 4), 2.41 (s 5) | 7.11 (m, 2H), 7.02 (d, J = 7.0 Hz, 1H), 6.87 (s, 4H), 5.82 (br s, 1H), 4.20 |
| | | (br s, 1H), 3.85-4.02 (m, 5H), 3.58-3.65 (m, 2H), 3.37-3.45 (m, 4H), |
| | | 3.08-3.19 (m, 1H) 2.91-3.06 (m 3H), 2.27 (s, 3H), 1.41-1.74 (m, 8H) |
| 62r | 82-85 | (DMSO-d₆) δ 8.17 (t, J = 5.5 Hz, 1H), 7.14 (d, J = 8.1 Hz, 2H), 7.09 (d, J = |
| | Rₜ: 2.26 (s | 8.0 Hz, 2H), 6.87 (s, 4H), 5.75 (br s, 1H), 4.02-4.11 (m, 1H), 3.99 (t, J = |
| | 4), 2.48 (s 5) | 4.8 Hz, 2H), 3.81-3.96 (m, 3H), 3.62 (t, J = 4.8 Hz, 2H), 3.38 (s, 2H), |
| | | 3.33-3.37 (m, 2H), 3.13 (dd, J = 12.7/3.2 Hz, 1H), 2.92-3.03 (m 3H), |
| | | 2.26 (s, 3H) 1.41-1.76 (m, 8H) |
| 62s | 110-113 | (DMSO-d₆) δ 8.10 (br s, 1H), 7.19 (t, J = 7.7 Hz, 1H), 6.80-6.90 (m, 6H), |
| | Rₜ: 2.21 (s | 6.78 (d, J = 8.3 Hz, 1H), 3.77-4.01 (m, 6H), 3.73 (s, 3H), 3.58-3.65 (m, |
| | 4), 2.41 (s 5) | 2H), 3.38, (s, 2H), 3.19 (dt, J = 5.8/5.8 Hz, 2H), 2.62-2.84 (m, 4H), 1.41- |
| | | 1.74 (m, 8H) |
| 62t | Rₜ: 2.19 (s | (DMSO-d₆) δ 7.90 (t, J = 5.3 Hz, 1H), 7.15 (d, J = 8.7 Hz, 2H), 6.79-6.90 |
| | 4), 2.37 (s 5) | (m, 6H), 4.90 (br s), 3.98 (t, J = 4.9 Hz, 2H), 3.75-3.96 (m, 4H), 3.70 (s, |
| | | 3H), 3.62 (t, J = 4.8 Hz, 2H), 3.31, (s, 2H), 3.11 (dt, J = 6.1/6.1 Hz, 2H) |
| | | 2.64 (dd, J = 11.9/4.3 Hz, 1H), 2.52-2.61 (m, 3H), 1.38-1.75 (m, 8H) |
| 62u | Decomp. | (DMSO-d₆) δ 8.33 (br s, 1H), 6.98-7.11 (m, 2H), 6.81-6.91 (m, 5H |
| | 140 | phenyl CH), 6.64-6.74 (m, 1H), 5.75 (br s, 1H), 4.09-4.21 (m, 1H), 3.85- |
| | Rₜ: 2.16 (s | 4.02 (m, 5H), 3.62 (t, J = 4.7 Hz, 2H), 3.36-3.45 (m, 4H), 2.86-3.13 |
| | 4), 2.33 (s 5) | (m, 4H) 1.41-1.76 (m, 8H) |
| 62v | 127.5-130 | (DMSO-d₆) δ 9.24 (s, 1H), 8.16 (t, J = 5.4 Hz, 1H), 7.04 (d, J = 8.5 Hz, 2H), |
| | Rₜ: 2.12 (s | 6.86 (s, 4H), 6.68 (d, J = 8.5 Hz, 2H), 5.56 (br s, 1H), 4.01-4.10 (m, 1H), |
| | 4), 2.27 (s 5) | 3.98 (t, J = 4.8 Hz, 2H), 3.90-3.96 (m, 1H) 3.87 (d, J = 5.2 Hz, 2H), 3.62 |
| | | (t, J = 4.8 Hz, 2H), 3.25-3.36 (m, 4H), 3.01 (dd, J = 12.2/3.6 Hz, 1H), 2.82- |
| | | 2.93 (m, 3H), 1.40-1.76 (m, 8H) |
| 62w | 99-102.5 | δ 8.03 (t, J = 5.5 Hz), 7.27 (d, J = 8.5 Hz, 2H), 7.02 (d, J = 8.6 Hz, 2H), |
| | Rₜ: 2.07 (s | 6.85 (s, 4H), 4.93 (br s, 1H), 3.75-4.01 (m, 6H), 3.62 (t, J = 4.8 Hz, 2H), |
| | 4), 2.26 (s 5) | 3.40 (s, 2H), 3.13 (dt, J = 6.1/6.1 Hz, 2H), 2.66 (dd, J = 11.8/4.2 Hz, 1H), |
| | | 2.53-2.63 (m, 3H), 2.25 (s, 3H), 1.40-1.76 (m, 8H) |

### General Method for the Preparation of the Aryloxyalkylamine Hydrochlorides (64a, c, d)

To a stirring suspension of polystyryldiphenylphosphine resine (PS-PPh₃, 1 g, 3 mmol, 1.5 equiv., from Aldrich, 1 g resin equivalent to 0.78 g of PPh₃), 63a, b (2.4 mmol, 1.2 equiv.), and substituted phenol (2 mmol, 1 equiv.) in DCM (10 mL) is added dropwise diisopropylazidocarboxylate (DIAD, 506 mg, 2.5 mmol, 1.25 equiv.). After overnight stirring the mixture is filtered through a silica plug and washed successively with DCM and a mix DCM/MeOH (1:1). The filtrate is then loaded on Isolute and purified by FCC (gradient petroleum ether/Ethyl Acetate 100:0 to 50:50) to obtain the intermediate Boc-protected aryloxyalkylamine, which is solubilised in dioxane (2 mL) and stirred during 3 hours in a 4M HCl solution in dioxane (8 mL). The mixture is then evaporated under reduced pressure, dried under high vacuum, and used as crude in the epoxide alkylation step.

### General Method for the Preparation of Aryloxyalkylamine Hydrochlorides (64e-f)

The first step (supported Mitsunobu reaction) is described in the synthesis of 64a, c, d. The intermediate Boc-protected p-benzyloxyphenoxyalkylamine obtained is then suspended in a mix DCM/EtOH (3:1, 10 mL) with Pd/C (10% w/w) and stirred overnight under a H₂ atmosphere. The suspension is then filtered through celite, evaporated under reduced pressure, and the residue is stirred 3 hours in a 4M HCl solution in dioxane (8 mL). The mixture is then evaporated under reduced pressure, dried under high vacuum, and used as crude in the epoxide opening step.

### General Method for the Preparation of Aryloxypropanolamines (65a, c-f)

41c (1 equiv.), crude aryloxyalkylamine hydrochloride 64a, c-f (2 equiv.), and NaOHₛ (2 equiv.) are suspended in hexafluoroisopropanol (HFIP, 4 mL by 100 mg of epoxide), and the mixture is stirred at 70° C. over 24 hours. The whole suspension is slowly wet-loaded at the top of a silica column and purified by FCC (eluent DCM/1M NH₃ in MeOH 90:10) to afford the corresponding aryloxypropanolamine 65a, c-f. Yields correspond to an isolated overall yield over the Mitsunobu reaction, amine deprotection, epoxide alkylation, and deprotection when applicable (10-55%).

Table 21 lists the ¹H NMR spectral data for selected compounds from Scheme 6:

| | m.p/° C.; | |
|---|---|---|
| Cpd | HPLC | ¹H NMR |
| 65a | white | (DMSO-d₆): δ 7.29 (t, J = 8.1 Hz, 1H, 5-H), 7.01 (t, J = 2.2 Hz, 1H, aryl 2-H), |
| | solid, mp | 6.97 (ddd, J = 8.1/2.2/0.8 Hz, 1H, aryl 6-H), 6.90 (ddd, J = 8.1/2.2/0.8 Hz, 1H, |
| | 72.5- | aryl 4-H), 6.83 (s, 4H, aryl-dioxy ring), 4.98 (br s, 1H, NH), 4.04 (t, J = 5.5 Hz, |
| | 73.5° C. | 2H, CH₂O-aryl-dioxy ring), 3.97 (t, J = 4.8 Hz, 2H, CH₂O-chlorophenyl), 3.95- |
| | Rₜ: 2.32 (s | 3.91 (m, 4H, ^{c}Pe CH, OCH₂CH(OH), CH(OH)), 3.62 (t, J = 4.8 Hz, 2H, |
| | 4), 2.51 (s | ^{c}PeOCH₂), 2.89 (t, J = 5.5 Hz, 2H, NHCH₂), 2.72 (dd, J = 11.7/3.9 Hz, 1H, |
| | 5) | CH(OH)CHHNH), 2.62 (dd, J = 11.7/3.9 Hz, 1H, CH(OH)CHHNH), 2.00 (br s, |
| | | 1H, OH), 1.76-1.42 (m, 8H, ^{c}Pe CH₂). |
| 65c | clear waxy | (CDCl₃): δ 7.52-7.49 (m, 2H, phenyl CH), 7.40-7.35 (m, 2H, phenyl CH), |
| | solid | 7.35-7.27 (m, 3H, phenyl CH, O-arylphenyl 3-H and 5-H), 7.05 (td, J = |
| | Rₜ: 2.39 (s | 7.5/1.0 Hz, 1H, O-arylphenyl 4-H), 6.99 (d, J = 8.0 Hz, 1H, O-arylphenyl 6-H), |
| | 4), 2.57 (s | 6.87-6.79 (m, 4H, aryl-dioxy ring), 4.09-4.05 (m, 4H, CH₂O-aryl-dioxy ring, |
| | 5) | CH₂O-arylphenyl), 4.01-3.97 (m, 1H, CH(OH)), 3.92-3.87 (m, 1H, ^{c}Pe |
| | | CH), 3.83 (dd, J = 5.2/0.7 Hz, 2H, OCH₂CH(OH)), 3.72 (t, J = 5.1 Hz, 2H, |
| | | ^{c}PeOCH₂), 2.94 (t, J = 5.1 Hz, 2H, NHCH₂CH₂O), 2.76 (dd, J = 12.1/3.8 Hz, |
| | | 1H, CH(OH)CHHNH), 2.65 (dd, J = 12.1/7.7 Hz, 1H, CH(OH)CHHNH), 2.19 |
| | | (br s, 2H, OH, NH), 1.79-1.50 (m, 8H, ^{c}Pe CH₂). |
| 65d | white | (CDCl₃): δ 7.57-7.50 (m, 4H, phenyl CH, O-arylphenyl CH meta to ether), |
| | solid, mp | 7.44-7.39 (m, 2H, phenyl CH), 7.30 (tt, J = 7.4/1.2 Hz, 1H, phenyl CH), 6.98 |
| | 154-155° C. | (d, J = 8.8 Hz, 2H, O-arylphenyl CH ortho to ether), 6.84 (s, 4H, aryl-dioxy |
| | Rₜ: 2.42 (s | ring), 4.12 (t, J = 5.2 Hz, 2H, CH₂O aryl-dioxy ring), 4.09-4.03 (m, 3H, |
| | 4), 2.63 (s | CH₂O-arylphenyl, CH(OH)), 4.01-3.94 (m, 3H, ^{c}Pe CH, OCH₂CH(OH)), |
| | 5) | 3.71 (t, J = 5.1 Hz, 2H, ^{c}PeOCH₂), 3.08 (t, J = 5.1 Hz, 2H, NHCH₂CH₂O), |
| | | 2.96 (dd, J = 12.1/3.8 Hz, 1H, CH(OH)CHHNH), 2.87 (dd, J = 12.1/7.7 Hz, |
| | | 1H, CH(OH)CHHNH), 2.25-1.49 (m, 10H, OH, NH, ^{c}Pe CH₂). |
| 65e | white | (DMSO-d₆): δ 8.89 (br s, 1H, PhOH), 6.84 (s, 4H, aryl-dioxy ring), 6.73 (d, J = |
| | solid, mp | 9.1 Hz, 2H, aryl CH ortho to phenol), 6.65 (d, J = 9.1 Hz, 2H, aryl CH ortho to |
| | 101-103° C. | phenol), 4.99 (br s, 1H, NH), 3.97 (t, J = 4.8 Hz, 2H, CH₂O-aryl-dioxy ring), |
| | Rₜ: 2.13 (s | 3.95-3.77 (m, 6H, CH₂O-hydroxyphenyl, ^{c}Pe CH, CH(OH), OCH₂CH(OH)), |
| | 4), 2.33 (s | 3.61 (t, J = 4.8 Hz, 2H, ^{c}PeOCH₂), 2.85 (t, J = 5.5 Hz, 2H, NHCH₂), 2.72 (dd, |
| | 5) | J = 11.7/3.9 Hz, 1H, CH(OH)CHHNH), 2.61 (dd, J = 11.7/3.9 Hz, 1H, |
| | | CH(OH)CHHNH), 2.31 (br s, 1H, OH), 1.70-1.40 (m, 8H, ^{c}Pe CH₂). |
| 65f | brown | (DMSO-d₆): δ 8.89 (br s, 1H, PhOH), 6.83 (s, 4H, aryl-dioxy ring), 6.72 (d, J = |
| | solid, mp | 9.1 Hz, 2H, aryl CH ortho to phenol), 6.64 (d, J = 9.1 Hz, 2H, aryl CH ortho to |
| | 133.5- | phenol), 4.95 (br s, 1H, NH), 3.97 (t, J = 4.8 Hz, 2H, CH₂O-aryl-dioxy ring), |
| | 136° C. | 3.95-3.77 (m, 6H, CH₂O-hydroxyphenyl, ^{c}Pe CH, CH(OH), OCH₂CH(OH)), |
| | Rₜ: 2.10 (s | 3.61 (t, J = 4.8 Hz, 2H, ^{c}PeOCH₂), 2.67 (m, 3H, CH(OH)CHHNH, |
| | 4), 2.29 (s | NHCH₂CH₂), 2.56 (dd, J = 11.7/6.2 Hz, 1H, CH(OH)CHHNH), 2.32 (br s, 1H, |
| | 5) | CH(OH)), 1.80 (quintuplet, J = 6.5 Hz, 2H, NHCH₂CH₂), 1.72-1.42 (m, 8H, |
| | | ^{c}Pe CH₂). |

### General Procedure for Synthesis of 2-aminoethoxybenzamide Hydrochlorides (66a-c)

63a (5.343 g, 33.14 mmol), the appropriate substituted hydroxybenzamide (5.00 g, 36.46 mmol, 1.1 eq) and Ph₃P (10.431 g, 39.77 mmol, 1.2 eq) were dispersed in THF (80 mL). DIAD (7.830 mL, 39.77 mmol, 1.2 eq) in THF (20 mL) was added dropwise, and the resulting mixtures stirred for 60 hours at rt. The mixtures were concentrated under reduced pressure, before dissolving in EtOAc (100 mL) and washing with aq. 2M NaOH (100 mL). The organic layer was then concentrated and further purified by FCC (gradient in DCM/EtOAc) to give white amorphous solids (yields 37-81%). The Boc-protected ethers were then dissolved or dispersed in DCM (30 mL) and MeOH (5-10 mL, where necessary to improve solubility) with stirring, before addition of an equal volume of 4M HCl/dioxane to solvent. After 2 hours 45 minutes of stirring at rt, the desired product was totally precipitated using excess PE, before collection by filtration (vacuum). To give the desired compounds as white amorphous solids (90-100% yield).

### General Procedure for Preparation of Aryloxypropanolamines (67a-e)

41c and the appropriate 66a-c, 2-(4-fluorophenoxy)ethylamine hydrochloride (66d, Alfa-Aesar, UK) or 2-(4-methoxyphenoxy)ethylamine (66e, Fisher Scientific, UK) were reacted according to the procedure described for the synthesis of 47w to give white amorphous solids after FCC purification (6-41% yield). As 66e is commercially available as the free amine, addition of tertiary base in the reaction was unnecessary. HPLC (S 4; s 5): 67a: 2.09; 2.25. 67b: 124.5-129.5: 2.11; 2.24. 67c: 128-131: 2.06; 2.21. 67d: 2.28; 2.50. 67e: 2.26; 2.46. 72: 121-122: 2.11; 2.29.

### 5-Acetyl-2-(benzyloxy)benzamide (69)

5-Acetyl-2-hydroxybenzamide (8.2 g, 45.76 mmol), K₂CO₃ (9.488 g, 68.65 mmol, 1.5 eq) and BnBr (8.609 g, 5.987 mL, 50.34 mmol, 1.1 eq) were heated under reflux in MeCN (100 mL) overnight. The mixture remained a white suspension throughout. After removal of MeCN under reduced pressure, the crude product was dispersed in water (100 mL), and extraction with EtOAc (50 mL) was attempted. The white precipitate was found to be insoluble in either layer, and so was collected by filtration (vacuum) to give 9.41 g of white solid. The aqueous layer was separated in the filtrate and washed with EtOAc (50 mL). Concentration of the combined organic layers gave a white solid with an odour of BnBr. This was sonicated in PE, before filtering, and washing with a small amount of DCM/PE (1:1). Total recovered yield of product: 12.077 g (98%).

### 2-(Benzyloxy)-5-hydroxybenzamide (70)

69 (5.00 g, 18.60 mmol) was dispersed in chloroform (50 mL) to give a white suspension which cleared on addition of m-CPBA 70-75% in water (6.86 g, 27.8 mmol, equivalent to 4.806 g). The mixture was stirred at rt for 24 hours, at which time LCMS analysis indicated partial conversion had taken place. A further 0.5 eq of m-CPBA were added and stirring was continued for a further 48 hours. The mixture was diluted with DCM (50 mL) before washing with sat. aq. NaHCO₃ (50 mL). The aqueous layer was washed with further DCM (2×50 mL) and the combined organic layers washed once more with NaHCO₃ (50 mL). The combined aqueous layers were found to be a yellow solution, whereas the organic layers formed a cloudy white suspension. This was solubilised by addition of a little MeOH, followed by drying with sodium sulphate. Concentration gave 7.1 g of crude product as a pale yellow oil which still contained aromatic impurities when analysed by 1H-nmr. Subsequently, the crude oil was dissolved in the minimum amount of EtOAc, before adding PE cautiously to cause precipitation of a white solid, which was collected by filtration (vacuum) and washed further with PE. Yield: 4.133 g, 78%.

The crude ester product (4.106 g, 14.49 mmol) and LiOH.H₂O (906 mg, 21.59 mmol, 1.5 eq) were weighed into a flask under an atmosphere of nitrogen gas. THF (25 mL) and water (25 mL) were added to form and initially yellow suspension, which darkens quickly). The mixture was stirred at rt for 4 hours 15 minutes, at which point a dark green solution had formed. THF was removed under reduced pressure, and the remaining aqueous slurry diluted with aq. 2M NaOH (25 mL) to form a complete solution. This was washed with EtOAc (25 mL). The basic aqueous layer was acidified with excess aq 2M HCl, before extraction with EtOAc (3×30 mL). After the third extraction, the aqueous layer remained dark, and the pH was found to be ˜7. Further 2M HCl solution was added, and the reacidifed aqueous layer extracted further with EtOAc (30 mL) after which is decolourised. The combined organic layers were concentrated to give a brown solid which did not require any further purification. Yield 3.261 g (93%).

### 5-(2-Aminoethoxy)-2-(benzyloxy)benzamide hydrochloride (71)

63a (1.205 g, 7.47 mmol), 70 (2.00 g, 8.22 mmol, 1.1 eq) and triphenylphosphine (2.156 g, 8.22 mmol, 1.1 eq) were dissolved in THF (40 mL) at rt. Diisopropylazodicarboxylate (1.618 mL, 8.22 mmol, 1.1 eq) was added slowly and the light brown solution became darker in colour. The mixture was stirred at rt overnight. After overnight stirring a further 1 equivalent of DIAD and PPh₃ were added. Stirring was continued for 5 days, before a further 1 eq of DIAD and PPh₃ were added. Stirring was continued overnight before stopping the reaction. The reaction mixture was concentrated under reduced pressure, and redissolved in EtOAc (100 mL), before washing with aq 2M NaOH (30 mL) and water (30 mL, addition of NaCl to aid separation). The organic layer was concentrated, and redissolved in EtOAc (30 mL), with addition of PE slowly, causing precipitation of triphenylphosphine oxide. This was removed by filtration (vacuum), and the filtrate concentrated and further purified by FCC to give two fractions:
Fraction 1: Desired compound and triphenylphosphine oxide 944 mg
Fraction 2: Same as fraction 1 with more impurity 1.083 g

Each fraction of contaminated ether product was reacted in a separate pot, being dissolved in DCM (10 mL) before addition of 4M HCl/dioxane (10 mL). The mixture was stirred for 2 hours, before addition of PE. However only a sticky solid was formed. TLC analysis (eluent 1M NH₃ in MeOH/DCM 15:85) indicated both reaction mixtures contained two spots, so the mixtures were combined before concentration and purification by FCC (eluent 1M NH₃ in MeOH/DCM 0:100 to 10:90) to give 790 mg of white crystalline solid (37% over 2 steps).

### 5-(2-(3-(4-(2-(Cyclopentyloxy)ethoxy)phenoxy)-2-hydroxypropylamino)ethoxy)-2-hydroxybenzamide (72)

41c and 71 were reacted according to the procedure described for the synthesis of 47w to give 90 mg of white amorphous solid after FCC purification (30% yield). The benzyl protected intermediate (60 mg, 0.11 mmol) was dissolved in THF (5 mL) and 10% Pd/C (6 mg) added. The mixture was hydrogenated overnight before filtering through a bed of celite and washing with MeOH. Concentration of the filtrate gave 52 mg of off-white amorphous solid (quantitative yield).

### Example A1

### Ligand Binding Studies

Selectivity of ligands for the three beta-adrenoceptors was assessed by whole-cell binding studies using ³H-CGP12177 in CHO cells expressing the human beta1, beta2 or beta3-adrenoceptors respectively essentially as described by Baker (2005; Br. J Pharmacol: 144, 317-22). Values shown are K_{D} values determined as described by Baker (2005). The K_{D} values for each ligand at the human beta1, beta2 and beta3 adrenoceptors are shown in Table 19. K_{D} represents the concentration of compound required to occupy 50% of the receptors in cells or tissues.

The selectivity of a ligand is given by the ratio of beta-1 to beta-2 K_{D}. Accordingly a difference of one in the logarithmic values thereof represents a 10-fold selectivity, a difference of 2 represents 100-fold selectivity and a difference of 3 represents 1000-fold selectivity etc.

TABLE 23
3H-CGP 12177 Whole cell binding

| | Beta 1 | | Beta 2 | | Beta 3 | |
|---|---|---|---|---|---|---|
| | Log K_{D} | n | Log K_{D} | n | Log K_{D} | n |
| 44a | −7.75 ± 0.04 | 10 | −5.60 ± 0.05 | 7 | −5.01 ± 0.07 | 7 |
| 44d** | −7.10 ± 0.07 | 3 | >−4 | 3 | >−4 COMPARISON | 3 |
| | | | | | (KNOWN) | |
| 45a | −7.15 ± 0.04 | 9 | * | 8 | −5.75 ± 0.06 | 6 |
| 45d | −7.04 ± 0.05 | 10 | * | 6 | * | 4 |
| 46a | −7.52 ± 0.03 | 9 | >−4 | 9 | −4.61 ± 0.05 | 4 |
| 46b | −8.50 ± 0.07 | 5 | −5.99 ± 0.04 | 6 | −5.95 ± 0.05 | 6 |
| 46d | −7.08 ± 0.04 | 12 | >−4 | 12 | * | 3 |
| 46e | −7.53 ± 0.06 | 6 | −5.24 ± 0.03 | 6 | −5.07 ± 0.15 | 6 |
| 46f | −7.50 ± 0.06 | 6 | −5.20 ± 0.04 | 6 | −5.14 ± 0.15 | 6 |
| 46g | −7.11 ± 0.06 | 6 | −6.27 ± 0.03 | 6 | −5.00 ± 0.14 | 6 |
| 46h | −7.35 ± 0.04 | 6 | −6.59 ± 0.05 | 6 | −5.03 ± 0.25 | 6 |
| 46i | −7.77 ± 0.07 | 6 | −6.03 ± 0.06 | 6 | −5.41 ± 0.14 | 6 |
| 46k | −7.67 ± 0.05 | 4 | −5.29 ± 0.05 | 3 | not tested | |
| 46l | −7.23 ± 0.01 | 4 | −4.81 ± 0.02 | 4 | not tested | |
| 46m | −7.79 ± 0.03 | 4 | −5.43 ± 0.05 | 3 | not tested | |
| 46n | −7.43 ± 0.04 | 4 | −4.87 ± 0.05 | 4 | not tested | |
| 46o | −7.86 ± 0.11 | 4 | −5.53 ± 0.02 | 4 | not tested | |
| 46p | −7.20 ± 0.04 | 4 | −4.59 ± 0.03 | 3 | not tested | |
| 47a | −7.90 ± 0.05 | 6 | −5.52 ± 0.03 | 6 | −4.62 ± 0.04 | 7 |
| 47b | −7.33 ± 0.03 | 6 | −6.27 ± 0.02 | 6 | −4.76 ± 0.08 | 6 |
| 47c | −8.04 ± 0.04 | 6 | −6.06 ± 0.03 | 6 | −4.76 ± 0.06 | 6 |
| 47d | −7.76 ± 0.04 | 6 | −5.80 ± 0.03 | 6 | −4.64 ± 0.04 | 6 |
| 47e | −7.02 ± 0.04 | 7 | −5.93 ± 0.02 | 8 | −4.86 ± 0.11 | 7 |
| 47f | −7.76 ± 0.03 | 7 | −6.05 ± 0.03 | 10 | −4.76 ± 0.09 | 7 |
| 47g | −7.80 ± 0.04 | 7 | −5.86 ± 0.04 | 5 | −4.70 ± 0.12 | 7 |
| 47h | −7.82 ± 0.03 | 6 | −5.94 ± 0.04 | 6 | −4.67 ± 0.03 | 6 |
| 47i | −8.17 ± 0.03 | 6 | −6.54 ± 0.02 | 6 | −4.73 ± 0.03 | 6 |
| 47j | −7.70 ± 0.04 | 5 | −5.92 ± 0.09 | 6 | −4.61 ± 0.06 | 6 |
| 47k | −7.11 ± 0.03 | 7 | −5.54 ± 0.03 | 5 | −4.73 ± 0.10 | 6 |
| 47l | −8.14 ± 0.04 | 6 | −5.59 ± 0.05 | 8 | −5.05 ± 0.09 | 8 |
| (R)-47l | −6.73 ± 0.04 | 4 | −5.23 ± 0.06 | 4 | not tested | |
| (S)-47l | −8.55 ± 0.03 | 4 | −5.94 ± 0.02 | 4 | not tested | |
| 47m | −7.95 ± 0.05 | 7 | −5.91 ± 0.86 | 9 | −4.87 ± 0.07 | 7 |
| 47n | −7.11 ± 0.01 | 5 | −6.10 ± 0.03 | 6 | −4.83 ± 0.03 | 6 |
| 47o | −7.92 ± 0.05 | 6 | −5.99 ± 0.04 | 6 | −4.99 ± 0.05 | 6 |
| 47p | −7.77 ± 0.04 | 6 | −5.86 ± 0.04 | 6 | −5.03 ± 0.07 | 6 |
| 47q | −6.94 ± 0.01 | 6 | −5.84 ± 0.02 | 6 | −4.62 ± 0.05 | 6 |
| 47r | −7.77 ± 0.06 | 6 | −5.86 ± 0.06 | 6 | −4.94 ± 0.10 | |
| 47s | −7.70 ± 0.04 | 6 | −5.76 ± 0.04 | 6 | −4.93 ± 0.09 | 6 |
| 47t | −6.99 ± 0.08 | 6 | −5.96 ± 0.05 | 6 | −5.40 ± 0.27 | |
| 47u | −7.89 ± 0.06 | 6 | −5.85 ± 0.06 | 6 | −5.16 ± 0.12 | 5 |
| 47v | −8.16 ± 0.08 | 8 | −5.45 ± 0.10 | 5 | −5.08 ± 0.08 | 5 |
| 47w | −7.73 ± 0.04 | 4 | −5.75 ± 0.02 | 4 | not tested | |
| 47x | −7.75 ± 0.03 | 3 | −5.74 ± 0.04 | 4 | not tested | |
| 47y | −7.91 ± 0.04 | 5 | −5.96 ± 0.04 | 5 | not tested | |
| 47z | −7.36 ± 0.03 | 4 | −5.69 ± 0.02 | 4 | not tested | |
| 47aa | −7.11 ± 0.02 | 4 | −4.36 ± 0.04 | 4 | not tested | |
| 47bb | −6.98 ± 0.03 | 7 | −4.36 ± 0.07 | 5 | not tested | |
| 48 | −7.46 ± 0.04 | 6 | −6.04 ± 0.02 | 6 | −5.35 ± 0.06 | 6 |
| 49 | −8.42 ± 0.03 | 6 | −7.27 ± 0.02 | 6 | −5.36 ± 0.12 | 6 |
| 51 | −7.53 ± 0.07 | 6 | −6.33 ± 0.03 | 7 | −5.98 ± 0.04 | 6 |
| 52 | −7.09 ± 0.06 | 7 | −6.16 ± 0.04 | 7 | −5.15 ± 0.10 | 6 |
| 53 | −7.59 ± 0.03 | 7 | −6.03 ± 0.04 | 7 | −4.72 ± 0.10 | 6 |
| 54 | −8.07 ± 0.09 | 6 | −5.59 ± 0.03 | 7 | −4.99 ± 0.05 | 6 |
| 54a | −8.02 ± 0.03 | 7 | −5.88 ± 0.03 | 7 | −5.05 ± 0.07 | 7 |
| 54b | −7.71 ± 0.10 | 6 | −5.82 ± 0.02 | 6 | not tested | |
| 54c | −8.14 ± 0.09 | 3 | −5.99 ± 0.07 | 4 | not tested | |
| 54d | −7.49 ± 0.07 | 6 | −6.30 ± 0.05 | 6 | −5.34 ± 0.04 | 5 |
| 54e | −7.51 ± 0.04 | 6 | −6.55 ± 0.03 | 6 | −5.96 ± 0.09 | 6 |
| 54f | −8.76 ± 0.05 | 6 | −6.79 ± 0.02 | 6 | −6.29 ± 0.11 | 6 |
| 54g | −6.69 ± 0.04 | 7 | −5.76 ± 0.04 | 7 | −5.51 ± 0.10 | 7 |
| 54h | −7.13 ± 0.06 | 7 | −6.26 ± 0.03 | 7 | −5.95 ± 0.12 | 7 |
| 54i | −7.56 ± 0.07 | 7 | −6.44 ± 0.03 | 7 | −5.69 ± 0.08 | 7 |
| 54j | −8.43 ± 0.04 | 6 | −7.81 ± 0.04 | 6 | −5.60 ± 0.09 | 5 |
| 60a | −7.12 ± 0.04 | 4 | −5.66 ± 0.05 | 4 | not tested | |
| 60b | −7.70 ± 0.02 | 4 | −5.38 ± 0.02 | 4 | not tested | |
| 60c | −7.53 ± 0.03 | 4 | −5.44 ± 0.02 | 4 | not tested | |
| 62a | −7.16 ± 0.01 | 4 | −5.94 ± 0.02 | 3 | not tested | |
| 62b | −7.75 ± 0.02 | 4 | −6.51 ± 0.03 | 4 | not tested | |
| 62c | −6.76 ± 0.02 | 4 | −5.35 ± 0.05 | 4 | not tested | |
| 62d | −7.86 ± 0.03 | 4 | −5.26 ± 0.05 | 4 | not tested | |
| 62e | −8.42 ± 0.02 | 4 | −6.27 ± 0.03 | 4 | not tested | |
| 62f | −8.31 ± 0.04 | 4 | −5.85 ± 0.03 | 4 | not tested | |
| 62g | −8.62 ± 0.04 | 4 | −7.01 ± 0.02 | 4 | not tested | |
| 62h | −8.17 ± 0.12 | 4 | −6.96 ± 0.03 | 4 | not tested | |
| 62i | −8.11 ± 0.03 | 4 | −6.75 ± 0.04 | 4 | not tested | |
| 62j | −8.46 ± 0.01 | 4 | −7.24 ± 0.01 | 4 | not tested | |
| 62k | −8.16 ± 0.02 | 4 | −6.87 ± 0.04 | 4 | not tested | |
| 62l | −8.12 ± 0.07 | 4 | −6.79 ± 0.05 | 4 | not tested | |
| 62m | −8.02 ± 0.04 | 4 | −6.90 ± 0.03 | 4 | not tested | |
| 62n | −7.88 ± 0.03 | 4 | −6.70 ± 0.02 | 4 | not tested | |
| 62o | −8.04 ± 0.02 | 4 | −6.67 ± 0.03 | 4 | not tested | |
| 62p | −8.02 ± 0.02 | 4 | −7.14 ± 0.02 | 4 | not tested | |
| 62q | −7.57 ± 0.03 | 4 | −6.52 ± 0.08 | 4 | not tested | |
| 62r | −7.30 ± 0.06 | 4 | −6.11 ± 0.05 | 4 | not tested | |
| 62s | −7.97 ± 0.04 | 4 | −7.27 ± 0.01 | 4 | not tested | |
| 62t | −8.01 ± 0.04 | 4 | −7.04 ± 0.07 | 4 | not tested | |
| 62u | −7.72 ± 0.02 | 4 | −6.86 ± 0.04 | 4 | not tested | |
| 62v | −8.27 ± 0.02 | 4 | −6.60 ± 0.03 | 4 | not tested | |
| 62w | −8.09 ± 0.03 | 4 | −6.59 ± 0.02 | 4 | not tested | |
| 65a | −7.13 ± 0.06 | 4 | −6.11 ± 0.04 | 4 | not tested | |
| 65c | −6.97 ± 0.04 | 4 | −6.13 ± 0.04 | 3 | not tested | |
| 65d | −6.40 ± 0.03 | 4 | −4.10 ± 0.06 | 3 | not tested | |
| 65e | −8.24 ± 0.04 | 4 | −6.69 ± 0.03 | 4 | not tested | |
| 65f | −7.46 ± 0.05 | 4 | −6.10 ± 0.04 | 4 | not tested | |
| 67a | −7.31 ± 0.07 | 4 | −5.98 ± 0.04 | 4 | not tested | |
| 67b | −7.74 ± 0.10 | 4 | −6.00 ± 0.03 | 4 | not tested | |
| 67c | −8.48 ± 0.07 | 4 | −6.07 ± 0.06 | 4 | not tested | |
| 67d | −8.03 ± 0.03 | 4 | −5.99 ± 0.04 | 4 | not tested | |
| 67e | −8.05 ± 0.10 | 4 | −6.10 ± 0.06 | 4 | not tested | |
| 72 | −9.11 ± 0.07 | 5 | −7.60 ± 0.03 | 5 | not tested | |
| Pindolo | −8.57 ± 0.03 | 8 | −9.23 ± 0.03 | 8 | −7.08 ± 0.08 | 7 |
| S-pindolo | −9.16 ± 0.09 | 5 | −9.55 ± 0.04 | 5 | −7.18 ± 0.15 | 4 |
| LK204-545 | −8.09 ± 0.04 | 8 | −5.20 ± 0.03 | 8 | −4.62 ± 0.09 | 7 |

**147-149 Eur J Med Chem 2002 37 731-741
*incomplete inhibition of specific binding at 0.01 mM
Values are mean ± s.e.mean of n separate experiments.

### Example A2

### Preliminary In Vivo Studies

Preliminary assessment of the action of one of the compounds of the invention was undertaken using an in vivo model for monitoring regional haemodynamics in conscious, freely-moving rats (Gardiner & Bennett, Am J Physiol. 1988 October; 255(4 Pt 2):H813-24). This model has the distinct advantage of enabling measurement of regional blood flow in addition to blood pressure in the fully conscious state. In this model, beta-adrenoceptor agonist administration elicits a beta2-adrenoceptor-selective hindquarters vasodilatation which, at higher doses, reduces arterial blood pressure and hence evokes a reflex tachycardia, the sympathetic component of which is beta1 adrenoceptor-mediated.

In atropine-treated rats (to remove the vagal component of the reflex tachycardia), pilot experiments (n=4) with 46a (10 mg/kg i.v.), using this experimental model, showed that the reflex heart rate response to salbutamol was abolished (before +55±14, after +7±4 beats/min) while the hypotension (before −11±1, after −11±3 mmHg) and increase in hindquarters vascular conductance (before +106±20, after +98±15%) were unaffected. Similarly, isoprenaline-induced tachycardia was markedly reduced (before +74±12, after +11±4 beats/min) while the hypotension (before −19±3, after −18±2 mmHg) and increase in hindquarters vascular conductance (before +126±20, after +129±19%) were unaffected. These data confirmed that 46a was highly beta1-adrenoceptor selective in a conscious rat cardiovascular model.

1. A compound of formula I-0, and its pharmaceutically acceptable salt or salts and physiologically hydrolysable derivatives in free form or salt form: 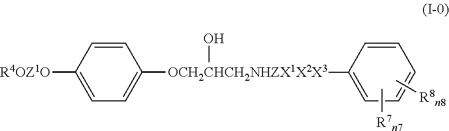
wherein
R⁴ is selected from unsubstituted and substituted C₃-C₈ cycloalkyl, C₁-C₈ linear or branched alkyl, C₂₋₅ alkenyl, C₆-C₁₀ heteroaryl or aryl, or C₃-C₈ heterocyclyl which may be part unsaturated, and combinations thereof;
Z¹ is C₁-C₄ linear or branched alkylene or alkenylene;
Z is linear C₂₋₃ alkylene;
X¹ is selected from NH and O;
X² is selected from unsaturated C and unsaturated S;
X³ is selected from NH and CH₂; or
one of X¹ and X³ is a single bond; or
X¹ is O and X² and X³ together are a single bond; and
R⁷ is selected from F, Cl, Br, CN, NH₂, NR⁹₂, NO₂, CF₃, OR⁹, COR⁹, OCOR⁹, COOR³, NR⁹COR⁹, CONR⁹₂ SO₂NR⁹₂, NR⁹SO₂R⁹; and
R⁸ is selected from C₁₋₅ alkyl, C₁₋₅ alkoxyl, C₂₋₅ alkenyl or alkynyl, C₆₋₁₀ aryl and C₃₋₈ cycloalkyl and combinations thereof, which may be unsubstituted or further substituted by one or more F, Cl, Br, CN, NH₂, NR⁹₂, NO₂, CF₃; and
R⁹ is selected from H and a group R⁸ as hereinbefore defined;
n7 and n8 and the sum thereof are independently selected from zero and the whole number integer 1 to 4;
with the proviso that it is not a compound as listed in the following Tables:
TABLE A1-0

| R⁴ | Z¹ | Z | X¹ | X² | X³ | R⁷ₙ₇, R⁸ₙ₈ |
|---|---|---|---|---|---|---|
| CH₃ | CH₂CH₂ | CH₂CH₂ | NH | CO | NH | — |
| c.prCH₂ | CH₂CH₂ | CH₂CH₂ | NH | CO | NH | — |
| p-OCH₃PhCH₂ | CH₂CH₂ | CH₂CH₂ | NH | CO | NH | — |

TABLE A3-0

| R⁴ | Z¹ | Z | X¹ | X² | X³ | R⁷ₙ₇, R⁸ₙ₈ |
|---|---|---|---|---|---|---|
| subst (e.g. | CH₂CH₂ | (CH₂)₂₋₅ | O | — | — | o-OH, m-CONR₂ |
| CH₃, | | (eg | | | | o-CONR₂, m-OH |
| c.prCH₂) | | (CH₂)₂) | | | | m-OH, p-CONR₂ |
| | | | | | | m-CONR₂, p-OH |
| | | | | | | where R is H or any |
| | | | | | | hydrocarbyl |
| | | | | | | eg CONH₂ CONHiBu or NR₂ |
| | | | | | | may form a cyclic moiety. |

TABLE A4-0

| R⁴ | Z¹ | Z | X¹ | X² | X³ | R⁷ₙ₇, R⁸ₙ₈ |
|---|---|---|---|---|---|---|
| c.prCH₂ | CH₂CH₂ | CH₂CH₂ | O | — | — | p-OCH₃ |
| p-CH₃OBz | CH₂CH₂ | CH₂CH₂ | O | — | — | p-OCH₃ |
| CH₃ | CH₂CH₂ | CH₂CH₂ | O | — | — | p-OCH₃ |
| CH₃ | CH₂CH₂ | CH₂CH₂ | O | — | — | m,p-(OCH₃)₂ |
| CH₃ | CH₂CH₂ | CH₂CH₂ | O | — | — | |
| CH₃ | CH₂CH₂ | CH₂CH₂ | O | — | — | m-CN, p-OH |

TABLE A6-0

| R⁴ | Z¹ | Z | X¹ | X² | X³ | R⁷ₙ₇, R⁸ₙ₈ |
|---|---|---|---|---|---|---|
| CH₃ | CH₂CH₂ | CH₂CH₂ | O | — | — | m-CONH₂, p-OH |
| c.prCH₂ | CH₂CH₂ | CH₂CH₂ | O | — | — | m,p-(OCH₃)₂ |

2. The compound as claimed in claim 1 wherein R⁴ is selected from unsubstituted and substituted C₃₋₇ cycloalkyl, C₃₋₇ cycloalkyl-C₀₋₃ alkyl, C₁₋₃ alkyl, C₆₋₁₀ aryl-C₀₋₃ alkyl, C₁₋₅alkoxy-C₆₋₁₀aryl-C₀₋₃ alkyl.

3. The compound as claimed in claim 1 wherein X² is selected from CO, CS, SO₂ and a single bond.

4. The compound as claimed in claim 1 wherein R⁷ and R⁸ are selected from R⁴OZ¹O as hereinbefore defined, m-,p-(OCH₃)₂ or o-, m- or p-OH, F, Cl, Br, NH₂, R³, OR³, or CF₃ or a combination thereof.

5. The compound as claimed in claim 1, wherein the compound is selected from a compound of formula IA⁰ and its pharmaceutically acceptable salt or salts and physiologically hydrolysable derivatives: 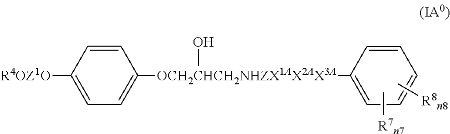
wherein
all integers are as hereinbefore defined; and
X^{1A} is NH;
X^{2A} is selected from unsaturated C and unsaturated S; and
X^{3A} is selected from NH and CH₂; or
one of X^{1A} and X^{3A} is a single bond;
with the proviso that it is not a compound as listed in Table A1A-0:
TABLE A1A-0

| R⁴ | Z¹ | Z | X¹ | X² | X³ | R⁷ₙ₇, R⁸ₙ₈ |
|---|---|---|---|---|---|---|
| CH₃ | CH₂CH₂ | CH₂CH₂ | NH | CO | NH | — |
| c.prCH₂ | CH₂CH₂ | CH₂CH₂ | NH | CO | NH | — |
| p-OCH₃PhCH₂ | CH₂CH₂ | CH₂CH₂ | NH | CO | NH | — |

6. The compound as claimed in claim 1, wherein the compound is selected from a compound of formula Ia-0 and its pharmaceutically acceptable salt or salts and physiologically hydrolysable derivatives: 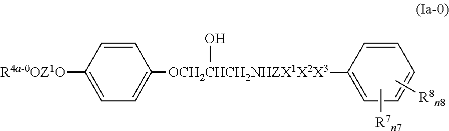
wherein
all integers are as hereinbefore defined; and
R^{4a-0} is optionally substituted C₃-C₈ cycloalkyl; more preferably is cyclopentyl.

7. The compound as claimed in claim 1, wherein the compound is selected from a compound of formula Ib-0 and its pharmaceutically acceptable salt or salts and physiologically hydrolysable derivatives: 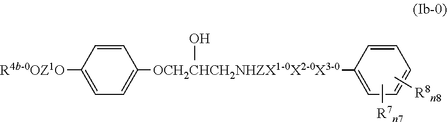
wherein
all integers are as hereinbefore defined; and
X¹⁻⁰ is NH, X²⁻⁰ is CO and X³⁻⁰ is selected from NH and CH₂ or one of X¹⁻⁰ and
X³⁻⁰ is a single bond;
R^{4b-0} is C₁-C₄ linear or branched alkyl-C₃-C₈ cycloalkyl; preferably is cyclopropylmethyl, more preferably remaining integers are as defined for formula 1A
with the proviso that it is not a compound as shown in the following table:
TABLE Alb-0

| R⁴ | Z¹ | Z | X¹ | X² | X³ | R⁷ₙ₇, R⁸ₙ₈ |
|---|---|---|---|---|---|---|
| c.prCH₂ | CH₂CH₂ | CH₂CH₂ | NH | CO | NH | — |

8. The compound as claimed in claim 1, wherein the compound is selected from a compound of formula Ic-0 and its pharmaceutically acceptable salt or salts and physiologically hydrolysable derivatives: 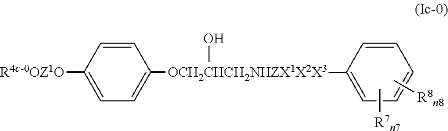
wherein
all integers are as hereinbefore defined; and
R^{4c-0} is ethyl.

9. The compound as claimed in claim 1, wherein the compound is selected from a compound of formula Id-0 and its pharmaceutically acceptable salt or salts and physiologically hydrolysable derivatives: 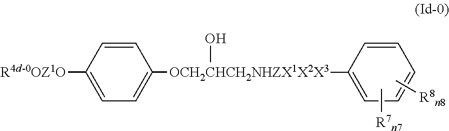
wherein
all integers are as hereinbefore defined; and
R^{4d-0} is optionally substituted C₆-C₁₀ aralkyl; more preferably is PhCH₂CH₂; most preferably substituted by one or more R⁷ as hereinbefore defined, for example F.

10. The compound as claimed in claim 1, wherein the compound is selected from a compound of formula Ie-0 and its pharmaceutically acceptable salt or salts and physiologically hydrolysable derivatives: 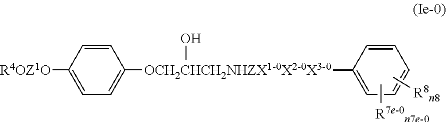
wherein
all integers are as hereinbefore defined; and
X¹⁻⁰ is NH, X²⁻⁰ is CO and X³⁻⁰ is selected from NH and CH₂ or one of X¹⁻⁰ and
X³⁻⁰ is a single bond;
R^{7e-0} is p-OH and n7e-0 is 1.

11. A compound of formula I-0 or subformulae as claimed in claim 1, as defined in the following Table 1-0:
TABLE 1-0

| Cpd | R⁴ | Z¹ | Z | X¹ | X² | X³ | R⁷ₙ₇, R⁸ₙ₈ |
|---|---|---|---|---|---|---|---|
| 45a | c.prCH2 | CH2CH2 | CH2CH2 | NH | C(═O) | NH | p-OCH2Ph |
| 45d | CH3CH2 | CH2CH2 | CH2CH2 | NH | C(═O) | NH | p-OCH2Ph |
| 46a | c.prCH2 | CH2CH2 | CH2CH2 | NH | C(═O) | NH | p-OH |
| 46b | p-FPh(CH2)2 | CH2CH2 | CH2CH2 | NH | C(═O) | NH | p-OH |
| 46d | CH3CH2 | CH2CH2 | CH2CH2 | NH | C(═O) | NH | p-OH |
| 46e | c.prCH2 | CH2CH2 | CH2CH2 | NH | C(═O) | NH | m-Cl |
| 46f | CH3CH2 | CH2CH2 | CH2CH2 | NH | C(═O) | NH | m-Cl |
| 46g | CH3CH2 | CH2CH2 | CH2CH2 | NH | C(═O) | CH2 | o-OH |
| 46h | CH3CH2 | CH2CH2 | CH2CH2 | NH | C(═O) | CH2 | m-OH |
| 46i | c.prCH2 | CH2CH2 | CH2CH2 | NH | C(═O) | CH2 | p-OH |
| 46k | iso-propyl | CH2CH2 | CH2CH2 | NH | C(═O) | NH | m-Cl |
| 46l | iso-propyl | CH2CH2 | CH2CH2 | NH | C(═O) | NH | p-OH |
| 46m | n-propyl | CH2CH2 | CH2CH2 | NH | C(═O) | NH | m-Cl |
| 46n | n-propyl | CH2CH2 | CH2CH2 | NH | C(═O) | NH | p-OH |
| 46o | c.pr | CH2CH2 | CH2CH2 | NH | C(═O) | NH | m-Cl |
| 46p | c.pr | CH2CH2 | CH2CH2 | NH | C(═O) | NH | p-OH |
| 47a | c.pent | CH2CH2 | CH2CH2 | NH | C(═O) | NH | — |
| 47b | c.pent | CH2CH2 | CH2CH2 | NH | C(═O) | NH | o-CH3 |
| 47c | c.pent | CH2CH2 | CH2CH2 | NH | C(═O) | NH | m-CH3 |
| 47d | c.pent | CH2CH2 | CH2CH2 | NH | C(═O) | NH | p-CH3 |
| 47e | c.pent | CH2CH2 | CH2CH2 | NH | C(═O) | NH | o-OCH3 |
| 47f | c.pent | CH2CH2 | CH2CH2 | NH | C(═O) | NH | m-OCH3 |
| 47g | c.pent | CH2CH2 | CH2CH2 | NH | C(═O) | NH | p-OCH3 |
| 47h | c.pent | CH2CH2 | CH2CH2 | NH | C(═O) | NH | o-F |
| 47i | c.pent | CH2CH2 | CH2CH2 | NH | C(═O) | NH | m-F |
| 47j | c.pent | CH2CH2 | CH2CH2 | NH | C(═O) | NH | p-F |
| 47k | c.pent | CH2CH2 | CH2CH2 | NH | C(═O) | NH | o-Cl |
| (R)- | c.pent | CH2CH2 | CH2CH2 | NH | C(═O) | NH | m-Cl |
| 47l | | | | | | | |
| (S)- | c.pent | CH2CH2 | CH2CH2 | NH | C(═O) | NH | m-Cl |
| 47l | | | | | | | |
| 47m | c.pent | CH2CH2 | CH2CH2 | NH | C(═O) | NH | p-Cl |
| 47n | c.pent | CH2CH2 | CH2CH2 | NH | C(═O) | NH | o-Br |
| 47o | c.pent | CH2CH2 | CH2CH2 | NH | C(═O) | NH | m-Br |
| 47p | c.pent | CH2CH2 | CH2CH2 | NH | C(═O) | NH | p-Br |
| 47q | c.pent | CH2CH2 | CH2CH2 | NH | C(═O) | NH | o-CF3 |
| 47r | c.pent | CH2CH2 | CH2CH2 | NH | C(═O) | NH | m-CF3 |
| 47s | c.pent | CH2CH2 | CH2CH2 | NH | C(═O) | NH | p-CF3 |
| 47t | c.pent | CH2CH2 | CH2CH2 | NH | C(═O) | NH | o-OH |
| 47u | c.pent | CH2CH2 | CH2CH2 | NH | C(═O) | NH | m-OH |
| 47v | c.pent | CH2CH2 | CH2CH2 | NH | C(═O) | NH | p-OH |
| 47w | c.pent | CH2CH2 | CH2CH2 | NH | C(═O) | NH | p-NO2 |
| 47x | c.pent | CH2CH2 | CH2CH2 | NH | C(═O) | NH | p-OCH2CH2F |
| 47y | c.pent | CH2CH2 | CH2CH2 | NH | C(═O) | NH | m-(C═O)OMe |
| 47z | c.pent | CH2CH2 | CH2CH2 | NH | C(═O) | NH | p-(C═O)OMe |
| 47aa | c.pent | CH2CH2 | CH2CH2 | NH | C(═O) | NH | -(C═O)OH |
| 47bb | c.pent | CH2CH2 | CH2CH2 | NH | C(═O) | NH | p-(C═O)OH |
| 48 | c.pent | CH2CH2 | CH2CH2 | O | — | — | — |
| 49 | c.pent | CH2CH2 | CH2CH2 | NH | C(═O) | CH2 | — |
| 50 | c.pent | CH2CH2 | CH2CH2CH2 | — | C(═O) | NH | — |
| 51 | c.pent | CH2CH2 | CH2CH2 | O | C(═O) | NH | — |
| 52 | c.pent | CH2CH2 | CH2CH2 | NH | C(=S) | NH | — |
| 53 | c.pent | CH2CH2 | CH2CH2 | NH | S(═O2) | CH2 | — |
| 54 | c.pent | CH2CH2 | CH2CH2 | NH | C(═O) | NH | m-F, p-OH |
| 54a | c.pent | CH2CH2 | CH2CH2 | NH | C(═O) | NH | m-F, p-F |
| 54b | c.pent | CH2CH2 | CH2CH2 | NH | C(═O) | NH | m-Cl, p-OMe |
| 54c | c.pent | CH2CH2 | CH2CH2 | NH | C(═O) | NH | m-Cl, p-OH |
| 54d | c.pent | CH2CH2 | CH2CH2 | NH | C(═O) | — | o-OH |
| 54e | c.pent | CH2CH2 | CH2CH2 | NH | C(═O) | — | m-OH |
| 54f | c.pent | CH2CH2 | CH2CH2 | NH | C(═O) | — | p-OH |
| 54g | c.pent | CH2CH2 | CH2CH2 | NH | C(═O) | — | o-F |
| 54h | c.pent | CH2CH2 | CH2CH2 | NH | C(═O) | — | m-F |
| 54i | c.pent | CH2CH2 | CH2CH2 | NH | C(═O) | — | p-F |
| 54j | c.pent | CH2CH2 | CH2CH2 | NH | C(═O) | CH2 | m-OH |
| 60a | c.pent | CH2CH2 | CH2CH2 | NH | C(═O) | NH | o-NH2 |
| 60b | c.pent | CH2CH2 | CH2CH2 | NH | C(═O) | NH | m-NH2 |
| 60c | c.pent | CH2CH2 | CH2CH2 | NH | C(═O) | NH | p-NH2 |
| 62a | c.pent | CH2CH2 | CH2CH2 | NH | C(═O) | — | — |
| 62b | c.pent | CH2CH2 | CH2CH2 | NH | C(═O) | — | o-NH2 |
| 62c | c.pent | CH2CH2 | CH2CH2 | NH | C(═O) | — | m-NH2 |
| 62d | c.pent | CH2CH2 | CH2CH2 | NH | C(═O) | — | p-NH2 |
| 62e | c.pent | CH2CH2 | CH2CH2 | NH | C(═O) | — | p-OAc |
| 62f | c.pent | CH2CH2 | CH2CH2 | NH | C(═O) | — | m-OMe, p-OH |
| 62g | c.pent | CH2CH2 | CH2CH2 | NH | C(═O) | CH2 | o-F |
| 62h | c.pent | CH2CH2 | CH2CH2 | NH | C(═O) | CH2 | m-F |
| 62i | c.pent | CH2CH2 | CH2CH2 | NH | C(═O) | CH2 | p-F |
| 62j | c.pent | CH2CH2 | CH2CH2 | NH | C(═O) | CH2 | o-Cl |
| 62k | c.pent | CH2CH2 | CH2CH2 | NH | C(═O) | CH2 | m-Cl |
| 62l | c.pent | CH2CH2 | CH2CH2 | NH | C(═O) | CH2 | p-Cl |
| 62m | c.pent | CH2CH2 | CH2CH2 | NH | C(═O) | CH2 | o-CF3 |
| 62n | c.pent | CH2CH2 | CH2CH2 | NH | C(═O) | CH2 | m-CF3 |
| 62o | c.pent | CH2CH2 | CH2CH2 | NH | C(═O) | CH2 | p-CF3 |
| 62p | c.pent | CH2CH2 | CH2CH2 | NH | C(═O) | CH2 | o-rH3 |
| 62q | c.pent | CH2CH2 | CH2CH2 | NH | C(═O) | CH2 | m-CH3 |
| 62r | c.pent | CH2CH2 | CH2CH2 | NH | C(═O) | CH2 | p-CH3 |
| 62s | c.pent | CH2CH2 | CH2CH2 | NH | C(═O) | CH2 | m-OMe |
| 62t | c.pent | CH2CH2 | CH2CH2 | NH | C(═O) | CH2 | p-OMe |
| 62u | c.pent | CH2CH2 | CH2CH2 | NH | C(═O) | CH2 | o-OH |
| 62v | c.pent | CH2CH2 | CH2CH2 | NH | C(═O) | CH2 | p-OH |
| 62w | c.pent | CH2CH2 | CH2CH2 | NH | C(═O) | CH2 | p-OAc |
| 65a | c.pent | CH2CH2 | CH2CH2 | O | — | — | m-Cl |
| 65c | c.pent | CH2CH2 | CH2CH2 | O | — | — | o-Ph |
| 65d | c.pent | CH2CH2 | CH2CH2 | O | — | — | p-Ph |
| 65e | c.pent | CH2CH2 | CH2CH2 | O | — | — | p-OH |
| 65f | c.pent | CH2CH2 | CH2CH2CH2 | O | — | — | p-OH |
| 67a | c.pent | CH2CH2 | CH2CH2 | O | — | — | o-(C═O)NH2 |
| 67b | c.pent | CH2CH2 | CH2CH2 | O | — | — | m-(C═O)NH2 |
| 67c | c.pent | CH2CH2 | CH2CH2 | O | — | — | p-(C═O)NH2 |
| 67d | c.pent | CH2CH2 | CH2CH2 | O | — | — | p-F |
| 67e | c.pent | CH2CH2 | CH2CH2 | O | — | — | p-OMe |
| 72 | c.pent | CH2CH2 | CH2CH2 | O | — | — | o-(C═O)NH2, p-OH |

12. A process for the preparation of a compound of formula I-0 or subformulae as defined in claim 1.

13. Novel intermediates of formula LIa, LIb, LIc, RIa, RIb, or of formula RIIa, RIIb, RIId, LIVa or LIVb:
R⁴OZ¹OPhOCH₂ oxirane (LIa)
R⁴OZ¹OPhOH (LIb)
R⁴OZ¹OPhOCH₂CH(OH)CH₂N(CH₂Ph)ZX¹X²OtBu (LIc)
HNHZX¹X²X³Ph (RIa)
oxirane-CH₂NHZX¹X²X³Ph (RIb)
tBuOCONHZX¹X²X³Ph (RIIa)
dioxoisoindoiineZX¹X²X³Ph (RIIb)
CH₂═CHCH₂NHZX¹X²X³Ph (RIId)
R⁴OZ¹O-tetrahydro-2H-pyran (LIVa)
R⁴OCH₂COOH (LIVb)
wherein
R⁴ is selected from unsubstituted and substituted C₃-C₈ cycloalkyl, C₁-C₈ linear or branched alkyl, C₂₋₅ alkenyl, C₆-C₁₀ heteroaryl or aryl, or C₃-C₈ heterocyclyl which may be part unsaturated, and combinations thereof;
Z¹ is C₁-C₄ linear or branched alkylene or alkenylene;
Z is linear C₂₋₃ alkylene;
X¹ is selected from NH and O;
X² is selected from unsaturated C and unsaturated S; and
X³ is selected from NH and CH; or one of X¹ and X³ is a single bond; or
X¹ is O and X² and X³ together are a single bond.

14. A composition comprising a therapeutically effective amount of a compound of formula I-0 or subformulae or its pharmaceutically acceptable salt and physiologically hydrolysable derivative as defined in claim 1 in association with one or more pharmaceutical carriers or diluents.

15. The use of a compound of formula I-0 or subformulae or pharmaceutically acceptable salt or composition as defined in claim 1 in the prevention or treatment of a condition selected from ischaemic heart disease, hypertension and heart failure, more preferably with concomitant respiratory disease, in particular asthma or COPD.

16. A method of treating a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, a compound of formula I-0 or subformulae or pharmaceutically acceptable salt or composition thereof as defined in claim 1 in an amount sufficient to treat the condition.

17. A method of treating a condition selected from ischaemic heart disease (also known as myocardial infarction or angina), hypertension and heart failure, restenosis and cardiomyopathy, more preferably with concomitant respiratory disease, in particular asthma or COPD, said method comprising administering to a subject in need thereof, the composition as defined in claim 14 in an amount sufficient to treat the condition.
